(19) United States
(12) Patent Application Publication
(10) Pub. No.: US 2007/0191349 A1
(43) Pub. Date: Aug. 16, 2007

(54) C8, c8' linked 5-oxo-1,2,3,11a-tetrahydro-5h-pyrrolo[2,1-c][1,4] benzodiazepine dimers with 1h-pyrrole-dicarboxylic acid amide linkers and oligomeric analogs therof as well as related compounds for the treatment of proliferative diseases

(75) Inventors: Philip Wilson Howard, Hertfordshire (GB); Stephen John Gregson, Greater London (GB); Arnaud Charles Tiberghien, Greater London (GB)

Correspondence Address: MICHAEL BEST & FRIEDRICH, LLP, ONE SOUTH PINCKNEY STREET, P O BOX 1806, MADISON, WI, 53701, US

(73) Assignee: SPIROGEN LIMITED, RYDE (GB)

(21) Appl. No.: 10/598,470

(22) PCT Filed: Mar. 1, 2005
(86) PCT No.: PCT/GB05/00767

§ 371 Date: Feb. 6, 2007

(30) Foreign Application Priority Data
Mar. 1, 2004 (GB) 0404578.7

(51) Int. Cl. A61K 31/551; C07D 487/14

(52) U.S. Cl. 514/220; 540/497

(57) ABSTRACT

Compounds of formula (I): PBD-A-Y—X—(Het)ₙₐ-L-(Het)_{nb}-L-(Het)_{nc}-T-(Het′)_{nd}-L-(Het′)ₙₑ-L-(Het′)_{nf}—X′—Y′- A′-PBD′ and salts, solvates and chemically protected forms thereof, are disclosed wherein the PBD units have the formulae (PBD) (PBD′) with the bonds at the 8 position on each molecule bond to the A and A′ groups respectively; A is selected from O, S, NH or a single bond, and each Het and Het′ is respectively an amino-heteroarylene-carbonyl group; X and X′ are both either NH or C (═O)-Q—C(═O)— wherein Q is a divalent group such that HY═R; in a second aspectm the invention comprises compounds of the general formula (II): PBD-A-Y—X—(Het)_{ng}-[L-(Het)ₙₕ]ₙⱼ-X′—Y′-A′-PBD′. Wherein: PBD and PBD′ are as defined above, X and X′ are either NH and C(═O) respectively or C(O) and NH respectively; the other substitutents are defined in the claims. Further aspects of the present invention relate to their use in the manufacture of a medicament for the treatment of a proliferative disease.

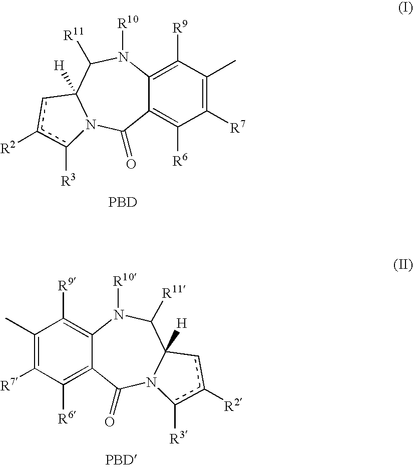

The present invention relates to pyrrolobenzodiazepines (PBDs) and in particular to PBD dimers and methods of synthesising PBD dimers.

### BACKGROUND TO THE INVENTION

The inventors have previously disclosed in WO 00/12508 PBD dimers which are PBD monomers joined at the 8-positions by a dioxyalkylene chain. These molecules exhibit a high level of cytotoxicity which arises due to the cross-linking of the two strands of DNA.

The inventors have also previously disclosed in WO 00/12506 the use of amino acids attached to a PBD monomer to attempt sequence selective binding of the molecule in the minor groove of DNA.

It has also been disclosed in the prior art that certain heterocylic amino acids can be used in the synthesis of hairpin polyamides which show some level of sequence selective interaction with DNA.

### DISCLOSURE OF THE INVENTION

The present inventors have developed a series of PBD dimer compounds with the chain linking the PBD monomer units comprising one or more amino-heteroarylene-carbonyl group.

In a first aspect, the invention comprises compounds of the general formula I:
PBD-A-Y—X—(Het)ₙₐ-L-(Het)_{nb}-L-(Het)_{nc}-T-(Het′)_{nd}-L-(Het′)ₙₑ-L-(Het′)_{nf}—X′—Y′-A′-PBD′
wherein: 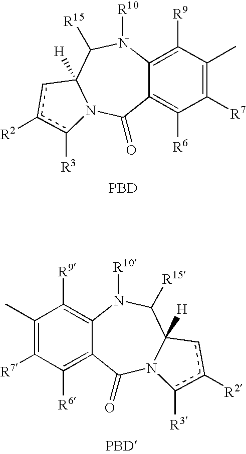
with the bonds at the 8 position on each molecule bond to the A and A′ groups respectively.
the dotted lines indicate the optional presence of a double bond between C1 and C2 or C2 and C3;
R² and R³ are independently selected from —H, —OH, ═O, ═CH₂, —CN, —R, OR, halo, ═CH—R, O—SO₂—R, CO₂R and COR;
R⁶, R⁷ and R⁹ are independently selected from H, R, OH, OR, SH, SR, NH₂, NHR, NRR′, nitro, Me₃Sn and halo;
where R and R′ are independently selected from optionally substituted C₁₋₇ alkyl, C₃₋₂₀ heterocyclyl and C₅₋₂₀ aryl groups; or
R⁶ and R⁷ together form a group —O—(CH₂)ₚ—O—, where p is 1 or 2;
R¹⁰ is a nitrogen protecting group and R¹⁵ is either O—R¹¹, wherein R¹¹ is a hydroxylprotecting group, or R¹⁵ is OH, ═O or ═S, preferably a hydroxylprotecting group or OH, or R¹⁰ and R¹⁵ together form a double bond between C10 and N11;
A is selected from O, S, NH or a single bond;
Y is a divalent group such that HY═R, or a single bond;
X and X′ are both either NH or C(═O); each Het and Het′ is independently an amino-heteroarylene-carbonyl group;
each L is independently selected from β-alanine, glycine, 4-aminobutanoic acid and a single bond;
T is a divalent linker group of the form:
—NH-Q-NH— or —C(═O)-Q-C(═O)—
wherein Q is a divalent group such that HQ=R;
A′, Y′, Het′, R^{2′}, R^{3′}, R^{6′}, R^{7′}, R^{9′}, R^{10′}, R^{11′}, R^{15′} and R^{15′} are all independently selected from the same lists as previously defined for A, Y, Het, R², R³, R⁶, R⁷, R⁹, R¹⁰, R¹¹ and R¹⁵ respectively;
na, nb, nc, nd, ne and nf are each independently 0 to 5 and the sum na+nb+nc+nd+ne+nf is 0 to 16.

In a second aspect, the invention comprises compounds of the general formula II:
PBD-A-Y—X—(Het)_{ng}-[L-(Het)ₙₕ]ₙⱼ-X′—Y′-A′-PBD′
Wherein: 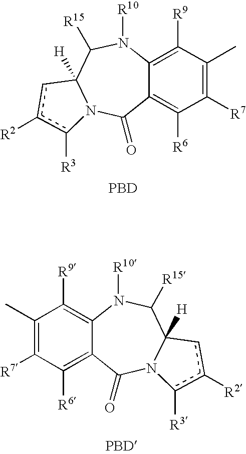
with the bonds at the 8 position on PBD and PBD′ bonding to the A and A′ groups respectively.
A, A′, Y, Y′, Het, L, R², R^{2′}, R³, R^{3′}, R⁶, R^{6′}, R⁷, R^{7′}, R⁹, R^{9′}, R¹⁰, R^{10′}, R¹¹, R^{11′}, R¹⁵ and R^{15′} are as previously defined;
ng is 1 to 5, nh is 1 to 5 and nj is 0 to 3
X and X′ are either NH and C(═O) respectively or C(═O) and NH respectively.

In a third aspect, the invention comprises a method of synthesis of the dimers of formula I or II.

Further aspects of the present invention relate to compounds of formula I or II (including solvates thereof when R¹⁰ and R¹⁵ form a double bond between N10 and C11, and pharmaceutical salts thereof), their use in methods of therapy (particularly in treating proliferative diseases), pharmaceutical compositions comprising these, and their use in the manufacture of a medicament for the treatment of a proliferative disease.

Definitions

The Het Amino-Heteroarylene-Carbonyl Group

The Het amino-heteroarylene-carbonyl group is of the general form:
-J-G-J′-
and the Het′ amino-heteroarylene-carbonyl group is of the general form:
-J′-G-J-
wherein J and J′ are either NH and C(═O) respectively or C(═O) and NH respectively and where when X is C(═O), J is NH and when X is NH, J is C(═O);
G is an optionally substituted heteroarylene group, preferably a C₅₋₁₆ heteroarylene group, more preferably a C₅₋₁₀ heteroarylene group and even more preferably a C₅₋₆ heteroarylene group. Furthermore in a preferred embodiment, the G group is a five membered heteroaryl group.

The heteroarylene group (G) may contain one or more heteroatoms and preferably contains one heteroatom. The one or more heteroatoms in the heteroarylene group (G) are independently chosen from N, O and S and are preferably N.

The heteroarylene group (G) is optionally substituted with one or more R groups. In a preferred embodiment the G group is substituted at one or more of the heteroatom positions with at least one R group, most preferably the R group is a methyl or ethyl group.

The J and J′ groups may be attached to the heteroarylene group (G) at any of the heteroarylene atoms, preferably the J and J′ groups are attached to the G group at two separate carbon atoms in the heteroarylene ring.

Where the G group is a six membered heteroarylene group, the J and J′ groups are preferably attached at the 2,6, 2,5, 3,6 or 3,5 positions.

Where the G group is a five membered heteroarylene group, the J and J′ groups are preferably attached at the 2,5, 2,4 or 3,5 positions.

Where the G group comprises two fused rings, the J and J′ groups are preferably attached to different rings.

Nitrogen Protecting Groups

Nitrogen protecting groups are well known in the art. Preferred nitrogen protecting groups are carbamate protecting groups that have the general formula:

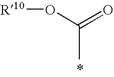

A large number of possible carbamate nitrogen protecting groups are listed on pages 503 to 549 of Greene, T. W. and Wuts, G. M., Protective Groups in Organic Synthesis, 3^{rd} Edition, John Wiley & Sons, Inc., 1999, which is incorporated herein by reference.

Particularly preferred protecting groups include Alloc, Troc, Teoc, BOC, Doc, Hoc, TcBOC, Fmoc, 1-Adoc and 2-Adoc.

Also suitable for use in the present invention are nitrogen protecting groups which can be removed in vivo (e.g. enzymatically, using light) as described in WO 00/12507, which is incorporated herein by reference. Examples of these protecting groups include: 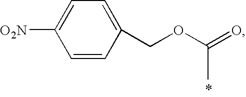
which is nitroreductase labile (e.g. using ADEPT/GDEPT); 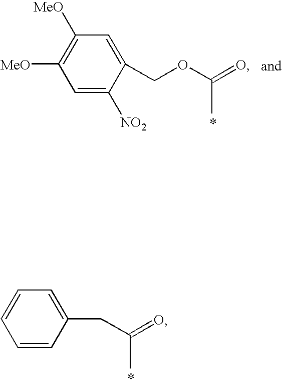
which are photolabile; and 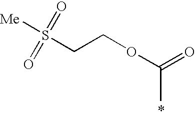
which is glutathione labile (e.g. using NPEPT).
Oxygen Protecting Groups

Oxygen protecting groups are well known in the art. A large number of suitable groups are described on pages 23 to 200 of Greene, T. W. and Wuts, G. M., Protective Groups in Organic Synthesis, 3^{rd} Edition, John Wiley & Sons, Inc., 1999, which is incorporated herein by reference.

Classes of particular interest include silyl ethers, methyl ethers, alkyl ethers, benzyl ethers, esters, benzoates, carbonates, and sulfonates.

Heteroarylene: The term heteroarylene, as used herein, pertains to a divalent moiety obtained by removing two hydrogen atoms from aromatic ring atoms of a heteroaromatic compound. Heteroarylene compounds as described herein correspond to heteroaryl groups as defined below with one fewer hydrogen atoms on the ring atoms. In addition, the heteroarylene groups as defined herein may be optionally substituted.

Substituents

The phrase “optionally substituted” as used herein, pertains to a parent group which may be unsubstituted or which may be substituted.

Unless otherwise specified, the term “substituted” as used herein, pertains to a parent group which bears one or more substitutents. The term “substitutent” is used herein in the conventional sense and refers to a chemical moiety which is covalently attached to, or if appropriate, fused to, a parent group. A wide variety of substitutents are well known, and methods for their formation and introduction into a variety of parent groups are also well known.

Examples of substitutents are described in more detail below.

C₁₋₇ alkyl: The term “C₁₋₇ alkyl” as used herein, pertains to a monovalent moiety obtained by removing a hydrogen atom from a carbon atom of a hydrocarbon compound having from 1 to 7 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

C₂₋₇ Alkenyl: The term “C₂₋₇ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₇ alkynyl: The term “C₂₋₇ alkynyl” as used herein, pertains to an alkyl group having one or more carbon-carbon triple bonds.

Examples of unsaturated alkynyl groups include, but are not limited to, ethynyl (ethinyl, —C≡CH) and 2-propynyl (propargyl, —CH₂—C≡CH).

C₃₋₇ cycloalkyl: The term “C₃₋₇ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:
saturated monocyclic hydrocarbon compounds:
   cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);
unsaturated monocyclic hydrocarbon compounds:
   cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and
saturated polycyclic hydrocarbon compounds:
   norcarane (C₇), norpinane (C₇), norbornane (C₇).

C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆ heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydro-pyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. Preferably, each ring has from 5 to 7 ring atoms.

In this context, the prefixes (e.g. C₃₋₂₀, C₅₋₇, C₅₋₆, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”. Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);
N₃: triazole (C₅), triazine (C₆); and,
N₄: tetrazole (C₅).

Examples of heteroaryl which comprise fused rings, include, but are not limited to:
C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ (with 2 fused rings) derived from benzodiazepine (N₂);
C₁₃ (with 3 fused rings) derived from carbazole (N₁), dibenzofuran (O₁), dibenzothiophene (S₁), carboline (N₂), perimidine (N₂), pyridoindole (N₂); and,
C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above groups, whether alone or part of another substitutent, may themselves optionally be substituted with one or more groups selected from themselves and the additional substitutents listed below.

Halo: —F, —Cl, —Br, and —I.

Hydroxy: —OH.

Ether: —OR, wherein R is an ether substitutent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇alkyl group.

Alkoxy: —OR, wherein R is an alkyl group, for example, a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR¹) (OR²), wherein R¹ and R² are independently acetal substitutents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe) (OEt).

Hemiacetal: —CH(OH) (OR¹), wherein R¹ is a hemiacetal substitutent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH)(OMe) and —CH(OH) (OEt).

Ketal: —CR (OR¹) (OR²), where R¹ and R² are as defined for acetals, and R is a ketal substitutent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me) (OMe)₂, —C(Me) (OEt)₂, —C(Me) (OMe) (OEt), —C(Et) (OMe)₂, —C(Et) (OEt)₂, and —C(Et) (OMe) (OEt).

Hemiketal: —CR(OH) (OR¹), where R¹ is as defined for hemiacetals, and R is a hemiketal substitutent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR, wherein R is an imino substitutent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substitutent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃ (t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substitutent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substitutent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O) Ph, and —OC(═O)CH₂Ph.

Oxycarboyloxy: —OC(═O)OR, wherein R is an ester substitutent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substitutents, for example, hydrogen, a C₁₋₇ alkyl group (also referred to as C₁₋₇ alkylamino or di-C₁₋₇alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substitutents, as defined for amino groups. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substitutents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substitutent, for example, hydrogen, a C₁₋₇ alkyl group, a C₃₋₂₀ heterocyclyl group, or a C₅₋₂₀ aryl group, preferably hydrogen or a C₁₋₇ alkyl group, and R² is an acyl substitutent, for example, a C₁₋₇ alkyl group, a C₃₋₂₀ heterocyclyl group, or a C₅₋₂₀ aryl group, preferably hydrogen or a C₁₋₇ alkyl group. Examples of acylamide groups include, but are not limited to, —NHC(═O)CH₃, —NHC(═O)CH₂CH₃, and —NHC(═O) Ph. R¹ and R² may together form a cyclic structure, as in, for example, succinimidyl, maleimidyl, and phthalimidyl:

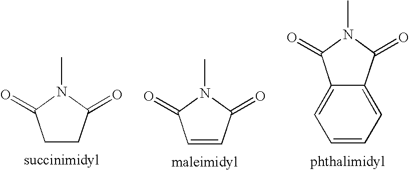

Aminocarbonyloxy: —OC(═O)NR¹R², wherein R¹ and R² are independently amino substitutents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹)CONR²R³ wherein R² and R³ are independently amino substitutents, as defined for amino groups, and R¹ is a ureido substitutent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

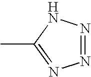

Imino: ═NR, wherein R is an imino substitutent, for example, for example, hydrogen, a C₁₋₇ alkyl group, a C₃₋₂₀ heterocyclyl group, or a C₅₋₂₀ aryl group, preferably H or a C₁₋₇alkyl group. Examples of imino groups include, but are not limited to, ═NH, ═NMe, and ═NEt.

Amidine (amidino): —C(═NR)NR₂, wherein each R is an amidine substitutent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substitutent, for example, a C₁₋₇ alkyl group (also referred to as a C₁₋₇alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substitutent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substitutent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substitutent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substitutent, for example, a C₁₋₇alkyl group, a C₃₋₂₀ heterocyclyl group, or a C₅₋₂₀ aryl group, preferably a C₁₋₇alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substitutent, for example, a C₁₋₇alkyl group, a C₃₋₂₀ heterocyclyl group, or a C₅₋₂₀ aryl group, preferably a C₁₋₇alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substitutent, for example, a C₁₋₇alkyl group, a C₃₋₂₀ heterocyclyl group, or a C₅₋₂₀ aryl group, preferably a C₁₋₇alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substitutent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substitutent, for example, a C₁₋₇ alkyl group, a C₃₋₂₀ heterocyclyl group, or a C₅₋₂₀ aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substitutents, as defined for amino groups. Examples of sulfamyl groups include, but are not limited to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substitutents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substitutent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substitutent, as defined for amino groups, and R is a sulfonamino substitutent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substitutent, as defined for amino groups, and R is a sulfinamino substitutent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substitutent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein R is a phosphinyl substitutent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O) (CH₃)₂, —P(═O) (CH₂CH₃)₂, —P(═O) (t-Bu)₂, and —P(═O) (Ph)₂.

Phosphonic acid (phosphono): —P(═O)(OH)₂.

Phosphonate (phosphono ester): —P(═O) (OR)₂, where R is a phosphonate substitutent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O) (OCH₃)₂, —P(═O) (OCH₂CH₃)₂, —P(═O) (O-t-Bu)₂, and —P(═O) (OPh)₂.

Phosphoric acid (phosphonooxy): —OP(═O)(OH)₂.

Phosphate (phosphonooxy ester): —OP(═O) (OR)₂, where R is a phosphate substitutent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphate groups include, but are not limited to, —OP(═O) (OCH₃)₂, —OP(═O) (OCH₂CH₃)₂, —OP(═O) (O-t-Bu)₂, and —OP(═O) (OPh)₂.

Phosphorous acid: —OP(OH)₂.

Phosphite: —OP(OR)₂, where R is a phosphite substitutent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited to, —OP(OCH₃)₂, —OP(OCH₂CH₃)₂, —OP(O-t-Bu)₂, and —OP(OPh)₂.

Phosphoramidite: —OP(OR¹)—NR²₂, where R¹ and R² are phosphoramidite substitutents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N (i-Pr)₂, and —OP(OCH₂CH₂CN)—N (i-Pr)_{2.}

Phosphoramidate: —OP(═O) (OR¹)—NR²₂, where R¹ and R² are phosphoramidate substitutents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O) (OCH₂CH₃)—N(CH₃)₂, —OP(═O) (OCH₂CH₃)—N(i-Pr)₂, and —OP(═O) (OCH₂CH₂CN)—N (i-Pr)₂.

Proliferative Diseases

One of ordinary skill in the art is readily able to determine whether or not a candidate compound treats a proliferative condition for any particular cell type. For example, assays which may conveniently be used to assess the activity offered by a particular compound are described in the examples below.

The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), leukemias, psoriasis, bone diseases, fibroproliferative disorders (e.g. of connective tissues), and atherosclerosis.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Methods of Treatment

As described above, the present invention provide the use of a compound of formula I or II in a method of therapy. Preferably the compounds of formulae I or II comprise a N10-C11 imine bond, or the N10 is protected by a nitrogen protecting group (R¹⁰) which can be removed in vivo and the C11 substitutent (R¹¹) is OH. Also provided is a method of treatment, comprising administering to a subject in need of treatment a therapeutically-effective amount of a compound of formula I or II, preferably in the form of a pharmaceutical composition, which is the third aspect of the present invention. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A compound may be administered alone or in combination with other treatments, either simultaneously or sequentially dependent upon the condition to be treated. Examples of treatments and therapies include, but are not limited to, chemotherapy (the administration of active agents, including, e.g. drugs; surgery; and radiation therapy. If the compound of formula I or II bears a carbamate-based nitrogen protecting group which may be removed in vivo, then the methods of treatment described in WO 00/12507 (ADEPT, GDEPT and PDT) may be used.

Pharmaceutical compositions according to the present invention, and for use in accordance with the present invention, may comprise, in addition to the active ingredient, i.e. a compound of formula I or II, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Includes Other Forms

Unless otherwise specified, included in the above are the well known ionic, salt, solvate, and protected forms of these substitutents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Isomers, Salts and Solvates

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Preferably compounds of the present invention have the following stereochemistry at the C11 position:

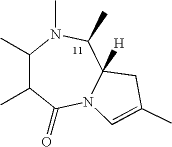

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

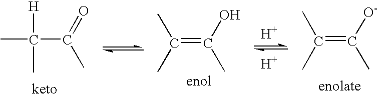

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylactic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

A particular salt form of interest can be formed from compounds of formula I and II, where R¹⁰ and R¹⁵ form an imine bond, by reacting said compound with a bisulphite salt to form a bisulphite derivative of the PBD. The PBD moieties of these compounds can be represented as: 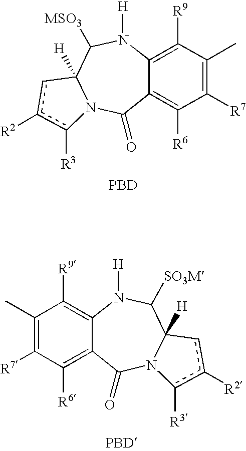
where M and M′ are independently monovalent pharmaceutically acceptable cations, or together form a divalent pharmaceutically acceptable cation, and the other groups are as previously defined.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

Solvates of particular relevance to the present invention are those where the solvent adds across the imine bond of the PBD moiety, which is illustrated below where the solvent is water or an alcohol (R^{A}OH, where R^{A} is an ether substitutent as described above):

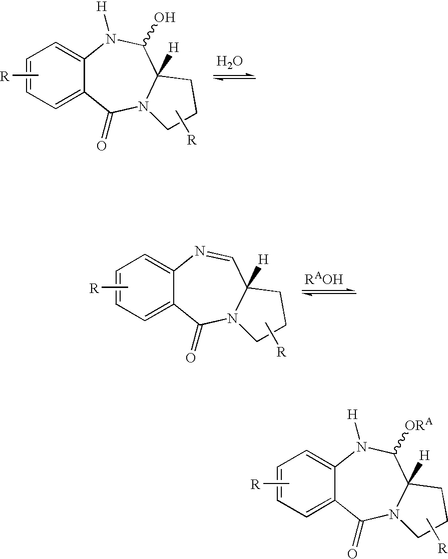

These forms can be called the carbinolamine and carbinolamine ether forms of the PBD. The balance of these equilibria depend on the conditions in which the compounds are found, as well as the nature of the moiety itself.

In general any nucleophilic solvent is capable of forming such solvates as illustrated above for hydroxylic solvents. Other nucleophilic solvents include thiols and amines.

These solvates may be isolated in solid form, for example, by lyophilisation.

General Synthetic Routes

For a general discussion of the synthetic routes used to obtain compounds of formulae I and II, the formulae I and II can each be conveniently divided into two capping groups joined by a linker group. The capping groups comprise the units PBD-A-Y—X— and —X′—Y′-A′-PBD′ and the linker groups comprise:
-(Het)ₙₐ-L-(Het)_{nb}-L-(Het)_{nc}-T-(Het′)_{nd}-L-(Het′)ₙₑ-L-(Het′)_{nf}-
in the case of formula I and:
-(Het)_{ng}-[L-(Het)ₙₕ]ₙⱼ-
in the case of formula II.

A key step in the synthesis of compounds of formula I or II is the linking of two capping groups with a linker group. In general the capping group may be joined to the linking group by a peptide bond of the form —NH—C(═O)—. This can be formed either by an amine terminated capping group reacting with a carboxylic acid (or equivalent) terminated linking group or vice versa (carboxylic acid (or equivalent) terminated capping group with an amine terminated linking group).
Reaction of an Amine Terminated Capping Group with a Carboxylic Acid (or Equivalent) Terminated Linking Group

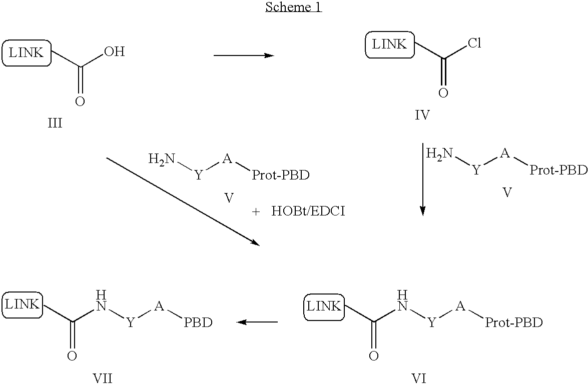

The generalised scheme 1 illustrates two possible methods for coupling an amine terminated capping group with a carboxylic acid (or equivalent) terminated linking group. In this scheme, only a mono-acidic linker compound III is shown. The linker group may contain additional functional groups or protected functional groups which may take part in further reactions of the product VII. It is also envisaged that the linker compound III may be a diacid. This would lead to a diacid chloride analogue of compound IV and ultimately to a product VII containing two capping groups joined by one linker group.

The first method proceeds by formation of the acid chloride (IV) of the carboxylic acid terminated linking compound (III). This may be achieved by reaction of III with oxalyl chloride. Compound IV is then reacted with the amine terminated capping group (V) in an elimination reaction to form a peptide bond.

Scheme 1 shows the coupling reaction proceeding via the acid chloride IV however it is envisaged that any activated carboxylic acid analogue of compound IV known in the art may alternatively be used in the coupling reaction.

The second coupling method proceeds without activation of the acid linking compound III. Instead the peptide coupling reaction proceeds directly with the amine terminated capping group in the presence of a coupling initiator. Preferred peptide coupling initiators may be chosen from BOP, BOP-Cl, DCC, DIC, FDPP, HATU, HOBt, PyBroP and TBTU. Preferably the coupling initiator is HOBt more preferably HOBt in conjunction with EDCI (as shown in scheme 1).

In both methods, the N10 position of the PBD group in the capping group (V) is preferably protected during the coupling reaction to avoid any unwanted side reactions. The group Prot-PBD is used to indicate that the N10 position on the PBD molecule is protected. Following the coupling reaction, the N10 protection on the PBD group in the capping group may be removed to yield the unprotected PBD dimer coupled to a linker unit (VII).

Furthermore, in the Prot-PBD group, the C11 hydroxyl group may optionally be protected during the coupling reaction. This may be achieved by using any hydroxylprotecting group known in the art, however, the C11 hydroxylprotecting group is preferably THP or a silyl ether (for example TBS).

The imine bond in the compound VI can be deprotected by standard methods to yield the unprotected compound (which may be in its carbinolamine or carbinolamine ether form, depending on the solvents used). For example if R¹⁰ in formula I or II is Alloc, then the preferred method of deprotection is hydrogenation using palladium on carbon to remove the N10 protecting group, followed by the elimination of water. If R¹⁰ is Troc, then the deprotection is carried out using a Cd/Pb couple to yield the compound VII.
Reaction of a Carboxylic Acid (or Equivalent) Terminated Capping Group with an Amine Terminated Linking Group

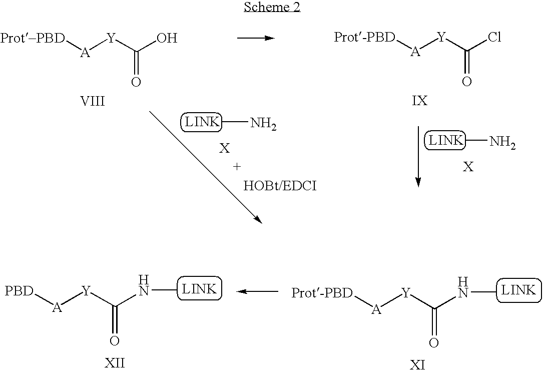

The generalised scheme 2 illustrates two possible methods for coupling a carboxylic acid (or equivalent) terminated capping group with an amine terminated linking group. In this scheme, only a mono-amine linker group X is shown. The linker group may contain additional functional groups or protected functional groups which may take part in further reactions of the product XII. It is also envisaged that the linker compound X may be a diamine. This would lead to a product XII containing two capping groups joined by one linker group.

Conditions for the coupling reactions in scheme 2 are as for those shown in scheme 1. It is important however that when the capping group VIII is carboxylic acid (or equivalent) terminated, the PBD group in compound VIII is protected at both the N10 and C11 positions. This is to avoid unwanted side reactions resulting in products other than compound XII. The group Prot′-PBD is used to indicate that the N10 and C11 positions of the PBD molecule are protected. The N10 nitrogen protecting group (R¹⁰) may be any nitrogen protecting group known in the art and is preferably a carbamate protecting group and is more preferably Alloc, Troc, Fmoc or Boc. The C11 oxygen protecting group may be any oxygen protecting group known in the art and is preferably THP or TBS.

Deprotection of compound XI at both the N10 and C11 positions (nitrogen and oxygen protection respectively) is achieved by methods known in the art.

In both scheme 1 and 2, if the nitrogen protecting group (R¹⁰) is such that the desired end product still contains it, e.g. if it is removable in vivo, then the C11 unprotected forms of compounds VII or XII above may be synthesised by removal of the oxygen protecting groups under suitable conditions to leave the R¹⁰ group unaffected.

Synthesis of Amine Terminated Capping Groups

As indicated above, the capping groups at each end of the linker chain may terminate either in an amine group or a carboxylic acid group (or equivalent). This allows coupling to a linker chain terminating in either an acid group or an amine group respectively by the methods shown above. Synthesis of an amine terminated capping group is shown generally in scheme 3:

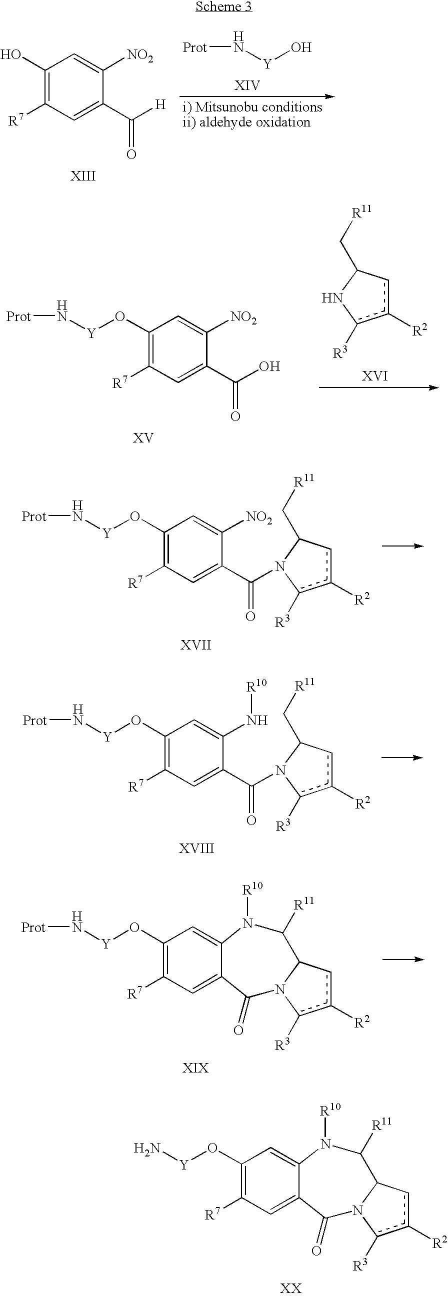

The starting material XIII (hydroxyl-5-substituted-2-nitro-benzaldehyde) may be coupled under Mitsunobu conditions, using, for example, PPh₃, to an N-protected hydroxy-amide (XIV). Subsequent oxidation of the aldehyde gives the corresponding acid (XV). The R⁷ group is as defined above but is preferably not reactive under Mitsunobu conditions nor susceptible to oxidation. The oxidation conditions used are known in the art but the reaction is preferably performed using hot aqueous KMnO₄.

The protected amine compound (XIV) may be BOC protected. However, any suitable amine protecting group known in the art may be used.

The attachment of a pre-formed PBD C-ring via peptide bonding and subsequent ring closure to form the PBD B-ring is known in the art and is demonstrated in WO 00/12508. Briefly, the PBD C-ring (as part of compound XVI) is attached to the compound XV via a peptide bond to give compound XVII. The NO₂ group in compound XVII is then reduced to the corresponding NH₂ group which is protected using any nitrogen protecting group known in the art. Preferably this protecting group is a carbamate protecting group and more preferably this is an alloc protecting group. The PBD B-ring is then formed by ring closure of the compound XVIII to give compound XIX. The protecting group may then be removed from the chain at the C8 position to give the desired amine terminated PBD capping group XX. An alternative method proceeding via an isocyanate intermediate is described in co-pending application GB 0321295.8.

The capping group XX may then be reacted with an acid terminated linker chain through the amine group of the chain at the C8 position to attach the capping group to the linker chain.

An alternative method of synthesis comprises synthesising a N10/C11 protected PBD unit with an 8-OH substitutent, and then coupling this, under Mitsunobu conditions, to the protected amine of formula XIV.

Synthesis of Carboxylic Acid (or Equivalent) Terminated Capping Groups

The carboxylic acid terminated linker compound (XXI) shown below is made from the known carboxylic acid ester (Tercel et al., J. Med. Chem., 2003, 46, 2132-2151):

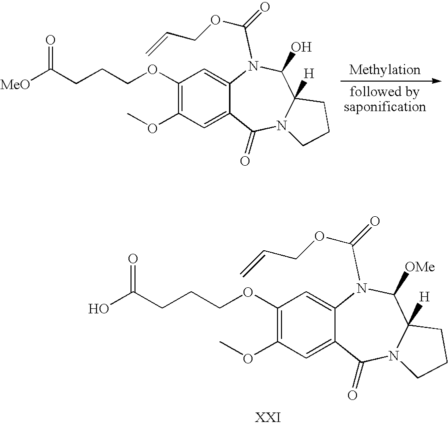

Methylation of the ester at the hydroxyl position on C11 followed by saponification of the methyl ester, preferably using NaOH, gives the corresponding acid terminated PDB capping group XXI which is protected at both the N10 and C11 positions.

It is envisaged that other acid terminated capping units could be formed by the same general synthesis strategy with different substitutents at the C7 position and/or a different chain at the C8 position, although still terminating in the methyl ester group and/or possible different N10 and C11 protecting groups and optionally substitutents at the C2 and/or C3 positions of the PBD C-ring.

The acid terminated capping group XXI can then be linked to an amine terminated linker group via a peptide bond. Where the substitutents present on the acid terminated capping group are different from those shown in XXI, it is important that they are stable under the coupling conditions used to link the capping group to the linking group. It is possible that alternative substitutent groups may be altered by the coupling reaction however unwanted side reactions should be avoided. It is clear to a person skilled in the art that careful choice of substitutent groups and protecting groups on the acid terminated capping group will ensure that unwanted side reactions are minimised.

Synthesis of Carboxylic Acid (or Equivalent) Terminated Linker Units

The simplest acid terminated linker units, which fall under the description of T above, are of the form HOOC-Q-COOH where Q is as defined above. In this case the units may be used directly to link two amine terminated capping units through the general coupling reactions shown in scheme 1. However, more complex linker units are envisaged that may be synthesised as follows:

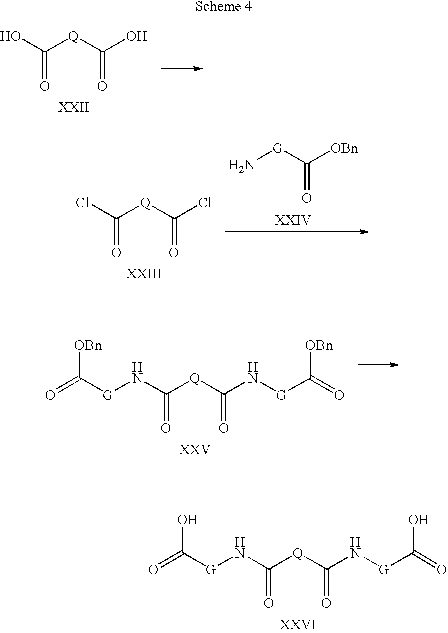

The diacid linker unit XXII may be coupled by peptide bonds to two acid-protected amine groups (XXIV) to form an acid protected dipeptide linker unit (XXV) that may subsequently be deprotected to form the diacidic linking unit XXVI.

Initial activation of the diacid XXII to form the acid chloride XXIII is preferably performed using oxalyl chloride. In scheme 4, activation of the diacid compound XXII is shown as proceeding via the acid chloride however any activation step known in the art may be used to form an active ester analogue of compound XXIII for reaction with the amine group on compound XXIV. Alternatively, a coupling initiator, as described above, could be used to couple the diacid XXII and the protected amine XXIV.

Importantly the acid group on compound XXIV is protected so that the peptide bond formation occurs favourably between the activated compound XXIII and the amine group on compound XXIV.

Following coupling, the dipeptide XXV may be deprotected to give the diacidic linker compound XXVI. Compound XXVI may then be used either to react with an amine functional group on an amine terminated capping unit to form a dimer of formula I, or to react with an amine functional group on a separate molecule to further lengthen the linker chain. For example reaction with a molecule of the general formula H₂N-G′-COOBn, in an analogous manner to the reaction shown in scheme 4, could be used to produce a linker molecule of the general formula:
HOOC-G′-NH—C(═O)-G-NH—C(═O)-Q-C(═O)—NH-G-C(═O)—NH-G′-COOH
(equivalent to:
HOOC-G′-NH-Het-T-Het′-NH-G′-COOH)

By repeated use of the general synthesis outlined in scheme 4, the skilled person could build up linker chains with a range of different G groups by successive addition of H₂N-Gⁿ′-COOBn molecules. These units could then be coupled in the same way as compounds of the formula XXV to acid terminated capping units (VIII) to form compounds of formula I.

The Gⁿ′ motif is used above to indicate that successive G groups in the linker unit need not necessarily be identical. It will be clear to the person skilled in the art that reaction with successive H₂N-Gⁿ′-COOBn groups in which the Gⁿ′ groups differ would result in linker chains formed comprising different heteroarylene groups.

Furthermore, orthogonal protection of both ends of the diacid compound XXII (or any diacid linker unit formed by addition of one or more H₂N-Gⁿ′-COOBn groups) followed by selective deprotection of one of the acid groups may allow linker units to be formed which do not have the same number of G units on each side of the T unit.

Preferably the number of Gⁿ′ units on each side of the T unit is between 0 and 8, more preferably between 0 and 5, more preferably between 0 and 3.

In the above discussion, the compound XXIV is shown with a Bn acid-protecting group. It is envisaged that any other acid-protecting group known in the art may be used as an alternative to the Bn group shown.

It is also envisaged that the Het units in the general formula I may be interrupted by spacer units (L) which alter the spacing between one Het group and the next. The identity of the possible L groups is as defined above i.e. β-alanine, glycine, 3-aminobutanoic acid (or a single bond, i.e. no L unit). Where L is an amino acid the addition of an L spacer group to the linker chain may be achieved as shown below:

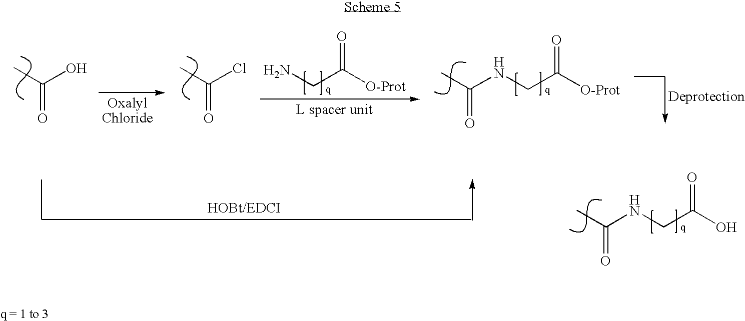

At any stage in the synthesis of the linker group wherein a compound is deprotected to give an acid, a spacer (L) group may be coupled to this acid by formation of a peptide bond. In scheme 5 above, two routes are shown to achieve this coupling, either via activation of the acid, to the acid chloride, followed by addition of the hydroxyl-protected spacer group or by direct peptide bond formation between the acid compound and the hydroxyl-protected spacer group. In both cases subsequent deprotection of the hydroxyl group results in the corresponding acid product.

As mentioned above, any activation step known in the art may be used to form an active ester analogue of the acid chloride compound. Also, any peptide bonding initiator known in the art may be used as an alternative to the HOBt/EDCI shown in scheme 5. Suitable peptide bonding initiator groups are as mentioned above.

This acid product may then be further coupled to a G group via a peptide bond in a similar manner to that shown in scheme 4. By using this general method of reacting an acid compound with an hydroxyl-protected amino acid followed by deprotection of the hydroxyl group, linker chains can be built up comprising Het groups and L spacer groups in various arrangements.

An example of a suitable hydroxylprotecting group is a Bn group. Preferably a spacer unit (L) is inserted into the linker chain after between 3 and 5 consecutive Het units. It is preferred that no more than 5 Het units occur consecutively in the linker chain without being interrupted by a spacer unit (L). More preferably no more than 3 Het units occur consecutively in the linker chain without being interrupted by a spacer unit (L).

The final deprotected acidic compound may then be coupled to an amine terminated capping unit (V) in a manner described in scheme 1 to give a product of formula I.

Synthesis of Amine Terminated Linker Units

The simplest amine terminated linker units, which fall under the description of T above, are of the form H₂N-Q-NH₂ where Q is as defined above. In this case the units may be used directly to link two carboxylic acid (or equivalent) terminated capping units. However, more complex linker units are envisaged that may be synthesised as follows:

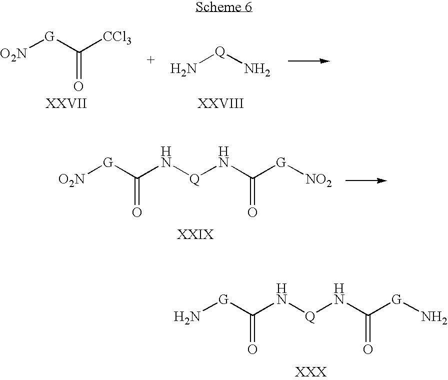

In scheme 6, the dipeptide compound XXIX is formed via an elimination reaction between compounds of the general formula XXVII and XXVIII. The two nitro groups on the dipeptide may then be reduced to form the diamine compound XXX. The reduction reaction is preferably a hydrogenation reaction performed with Pd/C and H₂ under pressure in a Parr apparatus.

The diamine XXX may then be used to react directly, under peptide coupling conditions, with carboxylic acid terminated capping units (VIII) to form compounds of formula I.

Alternatively, it is also envisaged that the compounds of formula XXX may be further reacted with other compounds to lengthen the linker chain. For example, reaction of compound XXX with a compound of the general formula O₂N-G′-C(═O)—CCl₃ could be used to give compounds of the general formula:
O₂N-G′-C(═O)—NH-G-C(═O)—NH-Q-NH—C(═O)-G-NH—C(═O)-G′-NO₂
(equivalent to:
O₂N-G′-C(═O)—Het-T-Het′-C(═O)-G′-NO₂)
Reduction of these molecules under conditions as described above may then give the corresponding diamine linker unit. These could then be coupled under peptide coupling conditions to carboxylic acid terminated capping units (VIII) to form a compound of formula I.

By repeated use of the general synthesis outlined in scheme 6, the skilled person could build up linker chains with a range of different G groups by successive addition of O₂N-Gⁿ′-C(═O)—CCl₃ molecules.

The Gⁿ′ motif is used above to indicate that successive G groups in the linker unit need not necessarily be identical. It will be clear to the person skilled in the art that reaction with successive O₂N-Gⁿ′-C(═O)—CCl₃ groups in which the Gⁿ′ groups differ would result in linker chains formed comprising different heteroarylene groups.

Furthermore, orthogonal protection of both ends of the diamine compound XXVIII (or any diamine linker unit formed by addition of one or more O₂N-Gⁿ′-C(═O)—CCl₃ groups) followed by selective deprotection of one of the amine groups may allow linker units to be formed which do not have the same number of G units on each side of the T unit.

Preferably the number of Gⁿ′ units on each side of the central T unit is between 0 and 5, more preferably 0 and 3.

In scheme 6 above, compound XXVII may be derived from a corresponding carboxylic acid by activation to substitute the activating CCl₃ group onto the acid. It is also envisaged that compound XXVII may be any other activated compound derived from a corresponding carboxylic acid, for example the acid chloride or acid bromide analogues. Also, the coupling reaction may be performed directly from the carboxylic acid from which XXVII is derived on reaction with compound XXVIII in the presence of a peptide coupling initiator as described above.

Furthermore, reaction of the compounds of formula XXX with a compound of the general formula ZO-C(═O)-G′-NHZ′ where Z and Z′ are oxygen and nitrogen protecting groups respectively could be used to give linker units as shown below:

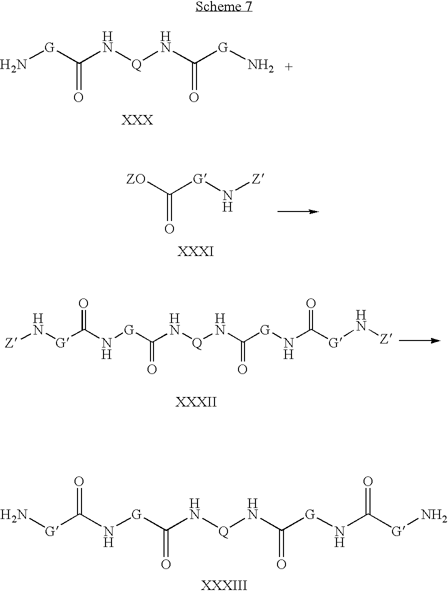

In scheme 7, Z is any oxygen protecting group known in the art although it is preferable that Z is removed under the peptide coupling conditions. Z may alternatively be an activating group derived from any peptide coupling reagent known in the art, for example BOP, BOP-Cl, DCC, DIC, EDPP, HATU, HOBt, PyBroP or TBTU, that activates compound XXXI to peptide coupling reactions. Preferably OZ is OBt, Cl or Br. Also Z′ is any nitrogen protecting group known in the art although it is preferable that Z′ is not removed under the peptide coupling conditions, more preferably Z′ is BOC, Fmoc, CBz, Alloc, Teoc, Adoc, Troc, Doc, Hoc or TcBOC. Removal of Z′ from compound XXXII to deprotect the diamine gives compound XXXIII. This may then be used as a linker unit to couple two acid terminated capping units (VIII) via peptide bond formation resulting in a compound of formula I.

It is also envisaged that the Het units in the general formula I may be interrupted by spacer units (L) which alter the spacing between one Het group and the next. The identity of the possible L groups is as defined above i.e. β-alanine, glycine, 3-aminobutanoic acid (or a single bond, i.e. no L group). Where L is an amino acid, the addition of an L spacer group to the linker chain may be achieved as shown below:

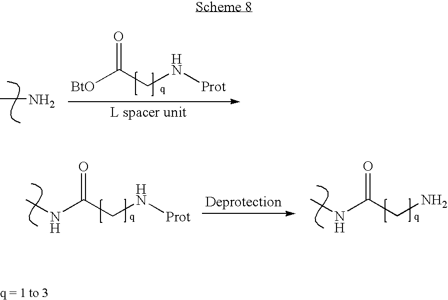

At any stage in the synthesis of the linker group wherein a compound is deprotected to give an amine, a spacer (L) group may be coupled to this amine by formation of a peptide bond. In scheme 8 above, this coupling is achieved by peptide bond formation between the amine compound and the nitrogen-protected spacer group. Subsequent deprotection of the nitrogen group results in the corresponding amine product. The hydroxyl group of the acid moiety on the spacer group may also be protected with a hydroxyl-protecting group which is removed under the peptide bond formation conditions.

Following coupling of the spacer unit to the amine compound and subsequent deprotection, the amine product may then be further coupled to a G group via a peptide bond in a similar manner to that shown in either scheme 6 or 7. By using this general method of reacting an amine compound with a amine-protected amino acid followed by deprotection of the amine group, linker chains can be built up comprising Het groups and L spacer groups in various arrangements.

The spacer unit (L) may be a β-alanine unit, a glycine unit or a 4-aminobutanoic acid unit.

The hydroxyl-protecting group in scheme 8 is shown as Bt. However any other hydroxyl-protecting group known in the art is also envisaged.

Preferably a spacer unit (L) is inserted into the linker chain after between 3 and 5 consecutive Het units. It is preferred that no more than 5 Het units occur consecutively in the linker chain without being interrupted by a spacer unit (L). More preferably no more than 3 Het units occur consecutively in the linker chain without being interrupted by a spacer unit (L).

The final unprotected amine compound may then be coupled to a carboxylic acid (or equivalent) terminated capping unit (VIII) in a manner described in scheme 2 to give a product of formula I.

Synthesis of Compounds of General Formula II

Compounds of the general formula II may be synthesised by the general synthetic route described below:

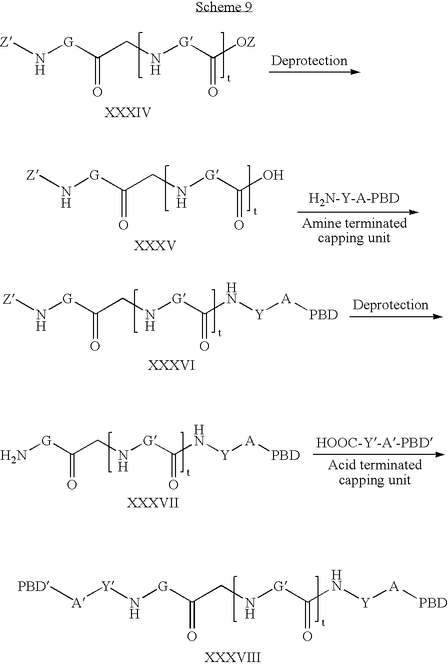

In this general scheme, compound XXXIV comprises at least one G unit, and may optionally comprise further G and/or spacer (L) units coupled together as shown above. In this scheme, t=0 to 12. Importantly, compound XXXIV has a nitrogen protected moiety and a hydroxyl protected moiety (Z′ and Z respectively).

Removal of the Z hydroxylprotecting moiety gives the unprotected acid compound XXXV. Preferably Z forms an ester functional group on compound XXXIV and more preferably Z is C₁₋₇ alkyl, even more preferably Z is methyl. In the preferred form where Z forms an ester functional group, hydrolysis of the ester under standard conditions yields the free acid compound XXXV.

Compound XXXV is then coupled with an amine terminated capping unit (V), via an elimination reaction forming a peptide bond, to give compound XXXVI.

Subsequently, the nitrogen protecting group (Z′) on compound XXXVI is removed to give the free amine compound XXXVII. Preferably the Z′ group is a carbamate nitrogen protecting group, more preferably Z′ is BOC. Where Z′ is BOC, HCl in dioxane is preferably used to cleave the BOC group to give the free amine.

The compound XXXVII is the coupled with an acid terminated capping unit (VIII), again via an elimination reaction forming a peptide bond, to give the compound XXXVIII.

The joining of the linker chain to the amine and carboxylic acid terminated end cap units could be carried out in the reverse order, with appropriate protection of the functional groups.

An alternative synthesis of compounds of formula II follows the method of WO 00/12509, where a PBD moiety is immobilised at the N10 position onto a solid support, and the linker chain is grown from the C8 position, using amino-heteroarylene-carbonyl groups as the combinatorial units. The chain grown at the C8 position can then be capped with the appropriate terminated capping unit.

In the above mentioned embodiments, the optionally substituted heteroarylene group (G) may alternatively be replaced with a C₅₋₆ arylene-C₅₋₆ arylene group or a C₈₋₁₀ heteroarylene-C₅₋₂₀ arylene group. C₅₋₆ arylene-C₅₋₆ arylene groups are as defined in copending application entitled “amino acids” filed on 1 Mar. 2004.

In the C₈₋₁₀ heteroarylene-C₅₋₂₀ arylene moiety described above, the C₈₋₁₀ heteroarylene group comprises two fused rings.

The term arylene, as used herein, pertains to a divalent moiety obtained by removing two hydrogen atoms from aromatic ring atoms of an aromatic compound having from 5 to 20 ring atoms. Arylene compounds as described herein correspond to aryl groups as defined above with one fewer hydrogen atoms on the ring atoms. Preferably, the C₅₋₂₀ arylene group is a C₅₋₇ arylene group and more preferably a C₅₋₆ heteroarylene group.

Het units comprising a carbonyl-C₈₋₁₀ heteroarylene-C₅₋₆ heteroarylene-amino unit have been described in Briehen, C. A., et al., Chem. Eur. J., 9, 2110-2122 (2003) and Renneberg, D., et al., J. Am. Chem. Soc., 125, 5707-5716 (2003) and include: 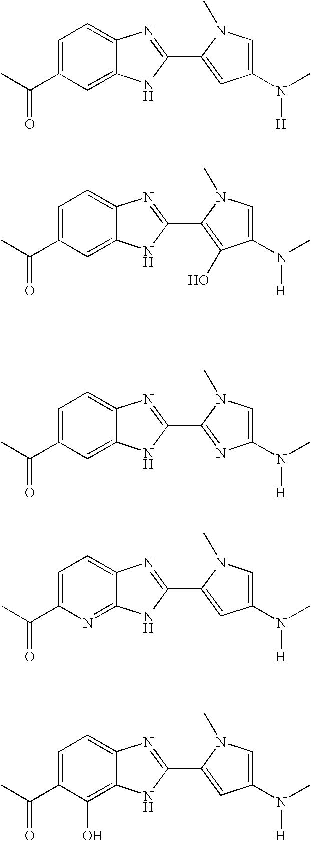
Further Preferences

The following preferences may apply to all aspects of the invention as described above, or may relate to a single aspect. The preferences may be combined together in any combination.

It is preferred that PBD and PBD′ are the same.

R⁹ is preferably H.

R² is preferably R, and is more preferably an optionally substituted C₅₋₂₀ aryl group. Most preferred is an optionally substituted phenyl group.

R⁶ is preferably selected from H, OH, OR, SH, NH₂, nitro and halo, and is more preferably H or halo, and most preferably is H.

R⁷ is preferably independently selected from H, OR, SH, SR, NH₂, NHR, NRR′, and halo, and more preferably independently selected from H and OR, where R is preferably selected from optionally substituted C₁₋₇ alkyl, C₃₋₁₀ heterocyclyl and C₅₋₁₀ aryl groups. Preferably R⁷ is OMe or H and most preferably OMe.

R¹⁰ is preferably H, BOC, Troc or alloc and is most preferably H or alloc.

R¹¹ is preferably THP or a silyl oxygen protecting group (for example TBS) and is most preferably THP.

In other embodiments of the invention, R¹⁰ and R¹⁵ together form a double bond between N10 and C11.

A is preferably NH, O or a single bond and most preferably NH or O.

Y is preferably a single bond or C₁₋₇ alkyl, more preferably a single bond or C₃ alkyl.

In the first aspect of the invention, Het and Het′ are preferably selected from the same class of amino-heteroarylene-carbonyl units.

A preferred class of amino-heteroarylene-carbonyl units are those based on nitrogen containing heteroarylene units, and in particular N-containing C5 heteroarylene units. These N-containing heteroarylene units are preferably substituted on one N atom with a C₁₋₄ alkyl group, which is more preferably methyl. A particularly preferred sub-class comprises the following three units:

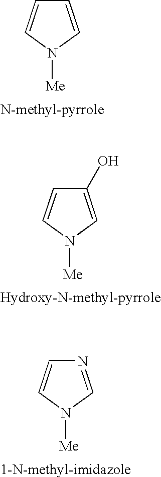

Other preferred units have heteroarylene groups based on 2-(pyrrol-2-yl)benzimidazoles, 2(pyrrol-2-yl)imiazopyridines and 5-hydroxy(pyrrol-2-yl)benzimadozles.

In the first aspect of the invention, the sums na+nb+nc and nd+ne+nf are preferably equal and are both more preferably between 1 and 3.

In the second aspect of the invention, the total number of Het groups in the compound (i.e. the sum ng+(nj×nh)) is preferably 1 to 3 and is more preferably 1 or 3.

### EXAMPLES

### Example 1

### Synthesis of Dimer 1 (SJG-605)

### a) 1-Methyl-1H-pyrrole-2,5-dicarboxylic acid bis-[(11S, 11aS)(11-hydroxy-7-methoxy-10-(carboxylic acid allyl ester)-5-oxo-1,2,3,10,11,11a-hexahydro-5H-pyrrolo[2,1-c][1,4]benzodiazepin-8-yl)-amide] (6).

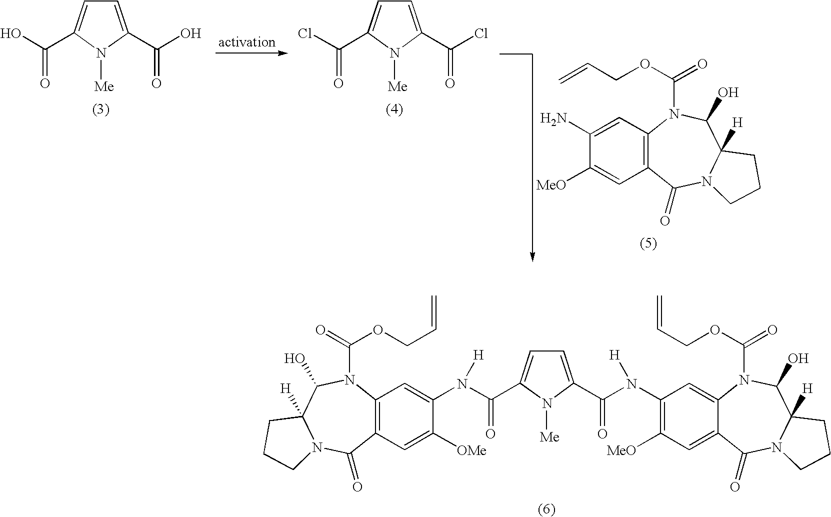

A catalytic amount of DMF (1 drop) was added to a stirred solution of the bis-acid 3 (49 mg, 0.29 mmol, J. Org. Chem., 43, 1978, 4849-53) and oxalyl chloride (81 mg, 55 μL, 0.63 mmol) in anhydrous THF (5 mL) at room temperature. Initial effervescence was observed and the mixture was allowed to stir for a further 4 h. The acid chloride solution was added dropwise to a solution of the aniline 5 (200 mg, 0.58 mmol, Bioorg. & Med. Chem. Lett., 13, 2003, 2277-80) and Et₃N (129 mg, 177 μL, 1.27 mmol) at 0° C. (ice/acetone) under a N₂ atmosphere. The reaction mixture was allowed to warm to room temperature and stirring was continued for 16 h. Analysis of the reaction mixture by TLC (90:10 v/v CHCl₃/MeOH) revealed amide formation. Excess THF was removed by rotary evaporation under reduced pressure and the resulting residue was dissolved in CH₂Cl₂ (30 mL). The organic phase was washed with saturated aqueous 1N HCl (3×10 mL), sat^{d} aqueous NaHCO₃ (10 mL), H₂O (10 mL), brine (10 mL) and dried (MgSO₄). The mixture was filtered and excess solvent removed by rotary evaporation under reduced pressure to afford the crude product as a thin film. The crude material was subjected to flash column chromatography (Neat CHCl₃ then 99:1 v/v CHCl₃/MeOH) and removal of excess eluent isolated the pure amide 6 as a white foam (161 mg, 68%): [α]²⁰_{D}=+38° (c=0.17, CHCl₃); ¹H NMR (400 MHz, CDCl₃) δ 8.46 (s, 2H), 8.44 (s, 2H), 7.30 (s, 2H), 6.71 (s, 2H), 5.92-5.75 (m, 2H), 5.74-5.60 (m, 2H), 5.24-5.08 (m, 4H), 4.67 (dd, 2H, J=13.1, 5.0 Hz), 4.59-4.46 (m, 2H), 4.26 (s, 3H), 4.00 (s, 6H), 3.77-3.69 (m, 2H), 3.61-3.47 (m, 4H), 2.22-1.96 (m, 8H); ¹³C NMR (100 MHz, CDCl₃) δ 166.8, 158.9, 156.2, 146.8, 132.0, 131.3, 129.9, 128.7, 128.5, 120.4, 117.9, 111.4, 109.5, 86.0, 66.9, 60.1, 56.4, 46.5, 34.8, 28.8, 23.1; IR (CHCl₃) 3417, 2976, 2954, 2879, 1707, 1689, 1628, 1609, 1589, 1519, 1480, 1460, 1430, 1411, 1380, 1340, 1313, 1275, 1249, 1200, 1039 cm⁻¹; MS (FAB) m/z (relative intensity) 850 ([M+Na]^{+.}, 21), 810 (15), 766 (23), 419 (33), 379 (100), 326 (67), 272 (41), 232 (79); HRMS [M+Na]^{+.} calcd for C₄₁H₄₅N₇O₁₂Na m/z 850.3024, found (FAB) m/z 850.2991.

### b) 1-Methyl-1H-pyrrole-2,5-dicarboxylic acid bis-[(11aS)(7-methoxy-5-oxo-1,2,3,11a-tetrahydro-5H-pyrrolo[2,1-c][1,4]benzodiazepin-8-yl)-amide] (1)

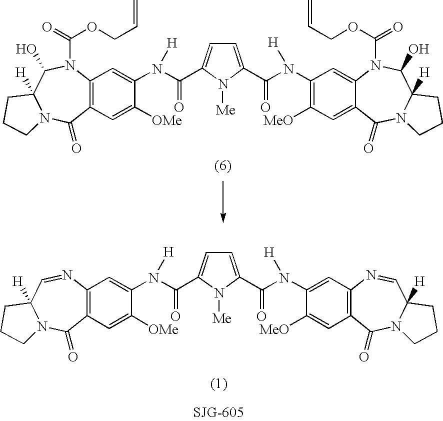

A catalytic amount of tetrakis(triphenylphosphine)palladium (9.9 mg, 8.5 μmol) was added to a stirred solution of the protected PBD (6) (141 mg, 0.17 mmol), Ph₃P (4.5 mg, 17.0 μmol) and pyrrolidine (25 mg, 30 μL, 0.36 mmol) in CH₂Cl₂ (10 mL) under a N₂ atmosphere. The reaction mixture was allowed to stir at room temperature and the progress of reaction monitored by TLC (90:10 v/v CHCl₃/MeOH), after 2.5 h the reaction was complete. The solvent was evaporated under reduced pressure and the resulting residue subjected to flash chromatography (98:2 v/v CHCl₃/MeOH) to give 1 (SJG-605) as a pale orange glass which was repeatedly evaporated in vacuo with CHCl₃ to provide the imine form (90 mg, 85%): [α]²⁰_{D}=+550° (c=0.45, CHCl₃); ¹H NMR (400 MHz, CDCl₃) δ 8.47 (s, 2H), 8.43 (s, 2H), 7.70 (d, 2H, J=4.4 Hz), 7.56 (s, 2H), 6.74 (s, 2H), 4.27 (s, 3H), 4.00 (s, 6H), 3.86-3.79 (m, 2H), 3.77-3.69 (m, 2H), 3.63-3.52 (m, 2H), 2.38-2.28 (m, 4H), 2.19-1.95 (m, 4H); ¹³C NMR (100 MHz, CDCl₃) δ 164.5, 162.6, 159.0, 146.2, 140.7, 131.2, 130.3, 122.6, 117.7, 111.5, 110.2, 56.3, 53.6, 46.7, 34.7, 29.6, 24.1; IR (CHCl₃) 3417, 2976, 2878, 1683, 1605, 1574, 1477, 1456, 1429, 1381, 1340, 1257, 1201, 1178, 1082, 1018 cm⁻¹; MS (FAB) m/z (relative intensity) 624 ([M+H]^{+.}, 100), 571 (10), 395 (15), 379 (63), 326 (23), 307 (54), 289 (29); HRMS [M+H]^{+.} calcd for C₃₃H₃₄N₇O₆ m/z 624.2571, found (FAB) m/z 624.2544.

### Example 2

### Synthesis of Dimer 2 (SJG-604)

### a) 1-Methyl-1H-pyrrole-2,4-dicarboxylic acid bis-[(11S, 11aS)(11-hydroxy-7-methoxy-10-(carboxylic acid allyl ester)-5-oxo-1,2,3,10,11,11a-hexahydro-5H-pyrrolo[2,1-c][1,4]benzodiazepin-8-yl)-amide] (9).

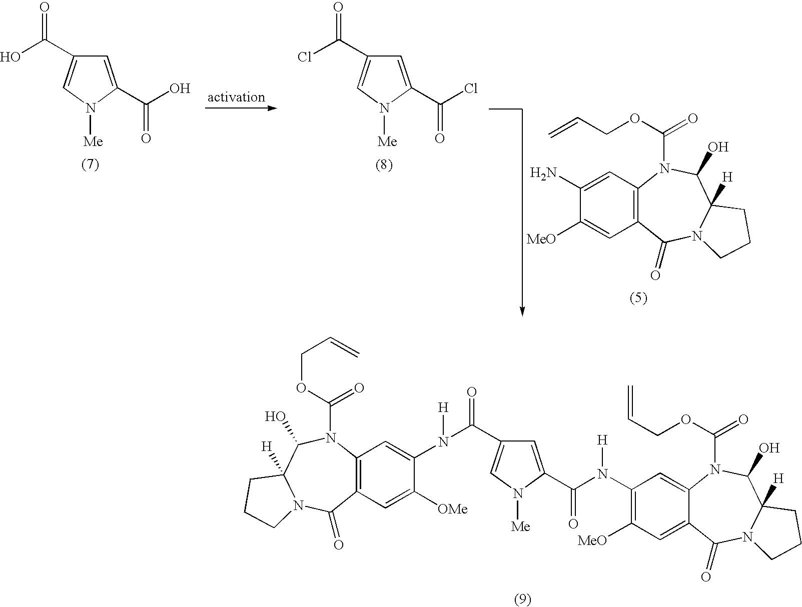

A catalytic amount of DMF (1 drop) was added to a stirred solution of the diacid (7) (49 mg, 0.29 mmol) and oxalyl chloride (81 mg, 55 μL, 0.63 mmol) in anhydrous THF (5 mL) at room temperature. Initial effervescence was observed and the mixture was allowed to stir for a further 15 min. The acid chloride solution was added dropwise to a solution of the aniline capping unit 5 (200 mg, 0.58 mmol, Bioorg. & Med. Chem. Lett., 13, 2003, 2277-80) and Et₃N (129 mg, 177 μL, 1.27 mmol) at 0° C. (ice/acetone) under a N₂ atmosphere. The reaction mixture was allowed to warm to room temperature and stirring was continued for 16 h. Analysis of the reaction mixture by TLC (90:10 v/v CHCl₃/MeOH) revealed amide formation. Excess THF was removed by rotary evaporation under reduced pressure and the resulting residue was dissolved in CH₂Cl₂ (30 mL). The organic phase was washed with saturated aqueous 1N HCl (3×15 mL), sat^{d} aqueous NaHCO₃ (15 mL), H₂O (15 mL), brine (15 mL) and dried (MgSO₄). The mixture was filtered and excess solvent removed by rotary evaporation under reduced pressure to afford the crude product as a thin film. The crude material was subjected to flash column chromatography (Neat CHCl₃ then 99:1 v/v CHCl₃/MeOH) and removal of excess eluent isolated the pure amide 9 as a white solid (168 mg, 71%): [α]²¹_{D}=+40° (c=0.15, CHCl₃); ¹H NMR (400 MHz, CDCl₃) δ 8.48-8.34 (m, 3H), 8.24-8.17 (m, 1H), 7.36 (s, 1H), 7.30 (s, 2H), 7.12 (s, 1H), 5.89-5.74 (m, 2H), 5.72-5.62 (m, 2H), 5.22-5.08 (m, 4H), 4.72-4.59 (m, 2H), 4.58-4.47 (m, 2H), 4.11-3.95 (m, 9H), 3.77-3.68 (m, 2H), 3.61-3.45 (m, 4H), 2.22-1.91 (m, 8H); ¹³C NMR (100 MHz, CDCl₃) δ 166.9, 166.8, 161.3, 158.7, 156.1, 146.9, 146.8, 132.0, 130.0, 129.8, 128.8, 128.6, 128.4, 128.2, 127.1, 120.8, 120.5, 119.0, 117.8, 111.5, 109.5, 86.0, 66.8, 60.1, 56.4, 46.5, 37.5, 28.8, 23.1; IR (CHCl₃) 3418, 2978, 2880, 1704, 1682, 1633, 1609, 1589, 1552, 1520, 1463, 1433, 1411, 1313, 1261, 1218, 1134, 1040 cm⁻¹; MS (FAB) m/z (relative intensity) 960 ([M+Cs]^{+.}, 100), 850 ([M+Na]^{+.}, 8), 464 (10), 419 (28); HRMS [M+Na]^{+.} calcd for C₄₁H₄₅N₇O₁₂Na m/z 850.3024, found (FAB) m/z 850.2991.

### b) 1-Methyl-1H-pyrrole-2,4-dicarboxylic acid bis-[(11aS)(7-methoxy-5-oxo-1,2,3,11a-tetrahydro-5H-pyrrolo[2,1-c][1,4]benzodiazepin-8-yl)-amide] (2)

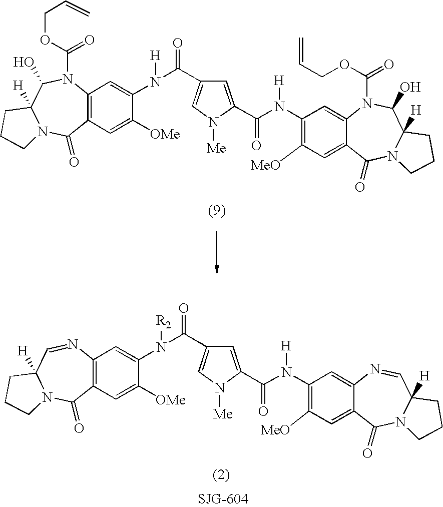

A catalytic amount of tetrakis(triphenylphosphine)palladium (10.9 mg, 9.4 μmol) was added to a stirred solution of the protected PBD (9) (156 mg, 0.19 mmol), Ph₃P (5.0 mg, 19.0 μmol) and pyrrolidine (28 mg, 33 μL, 0.40 mmol) in CH₂Cl₂ (10 mL) under a N₂ atmosphere. The reaction mixture was allowed to stir at room temperature and the progress of reaction monitored by TLC (90:10 v/v CHCl₃/MeOH), after 2 h the reaction was complete. The solvent was evaporated under reduced pressure and the resulting residue subjected to flash chromatography (98:2 v/v CHCl₃/MeOH) to give 2 (SJG-604) as a pale orange glass which was repeatedly evaporated in vacuo with CHCl₃ to provide the imine form (99 mg, 84%): [α]¹⁹_{D}=+433° (c=0.49, CHCl₃); ¹H NMR (400 MHz, CDCl₃) δ 8.38 (s, 2H), 8.30 (s, 1H), 8.21 (s, 1H), 7.61 (d, 2H, J=4.3 Hz), 7.47 (s, 1H), 7.46 (s, 1H), 7.33 (d, 1H, J=1.65 Hz), 7.11 (d, 1H, J=1.75 Hz), 3.94-3.92 (m, 9H), 3.80-3.70 (m, 2H), 3.69-3.59 (m, 2H), 3.55-3.43 (m, 2H), 2.30-2.11 (m, 4H), 2.09-1.85 (m, 4H); ¹³C NMR (100 MHz, CDCl₃) δ 163.5 (2 signals), 161.6, 161.5, 160.4, 157.9, 145.3, 145.1, 139.7, 139.6, 129.6, 129.3, 129.1, 128.9, 126.0, 121.6, 121.3, 117.9, 116.9, 116.8, 110.6, 109.3, 109.1, 55.3, 52.7, 45.7, 36.5, 28.6, 23.2; IR (CHCl₃) 3417, 2975, 2877, 1667, 1605, 1575, 1552, 1510, 1480, 1463, 1340, 1260, 1214, 1179, 1020 cm⁻¹; MS (FAB) m/z (relative intensity) 640 ([M+Na]^{+.}, 9), 624 ([M+H]^{+.}, 63), 592 (11), 395 (20), 379 (100), 365 (9), 326 (15), 307 (23); HRMS [M+H]^{+.} calcd for C₃₃H₃₄N₇O₆ m/z 624.2571, found (FAB) m/z 624.2544.

### Example 3

### Synthesis of an Amino Capping Unit 10

### a) 4-(3-tert-Butoxycarbonylamino-propoxy)-5-methoxy-2-nitro-benzoic Acid (35)

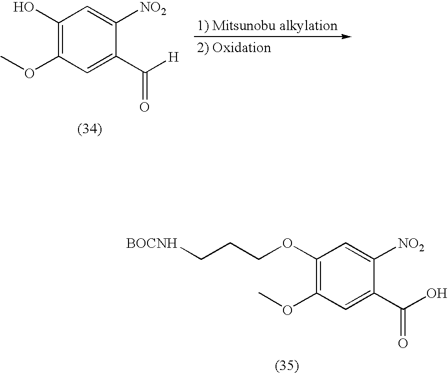

A solution of diethylazodicarboxylate (16 mL, 0.10 mol) in anhydrous THF (200 mL) was added dropwise to a stirred solution of 4-Hydroxy-5-methoxy-2-nitro-benzaldehyde (20 g, 0.10 mol J. Am. Chem. Soc., 112, 1990, 7050-51), (3-Hydroxy-propyl)-carbamic acid tert-butyl ester (17.35 mL, 0.10 mol), and triphenylphosphine (26.63 g, 0.10 mol) in anhydrous THF (800 mL). The reaction mixture was allowed to stir overnight after which time the solvent was removed in vacuo. The residue was triturated with toluene (500 mL) and filtered. The resulting filtrate washed with 1N aqueous NaOH (300 mL), brine (300 mL), dried (MgSO₄), filtered and evaporated in vacuo. The residue was triturated with diethyl ether (400 mL) and the precipitate was collected by filtration and then air dried to yield a pale yellow solid (20 g), which contained the desired product contaminated by some side-reaction products. This batch was used directly in the next step without any further purification. The solid was dissolved in acetone (200 mL) and vigorously stirred. A hot solution (85° C.) of KMnO₄ (20 g) in water (200 mL) was added dropwise to the mixture where vigorous reflux occurred. The reaction mixture was stirred for 1 h after which time it was filtered through celite. The acetone was removed in vacuo and the remaining aqueous phase was basified to pH 10 with 1N aqueous sodium hydroxide. The solids were removed by filtration and the filtrate washed with EtOAc (400 mL). The aqueous phase was acidified to pH 2-3 with concentrated aqueous citric acid and the resulting suspension was extracted with EtOAc (400 mL), washed with brine (100 mL), dried (MgSO₄), filtered and the solvent was removed in vacuo to yield 35 (12 g, 32% over two steps) as a dark oil that solidifies in the freezer. A sample was recrystallised from EtOAc to yield a white solid, which provided analytical data: ¹H NMR (400 MHz, DMSO-d₆) δ 7.56 (s, 1H), 7.30 (s, 1H), 6.91 (t, J=5 Hz, 1H), 4.11 (t, J=6.0 Hz, 2H), 3.92 (s, 3H), 3.10 (q, J=6.0 Hz, 2H), 1.86 (p, J=6.3 Hz, 2H), 1.38 (s, 9H); ¹³C NMR (100 MHz, DMSO-d₆) δ 166.0, 155.6, 151.7, 149.4, 141.3, 121.0, 111.2, 107.8, 77.5, 66.9, 56.4, 36.8, 28.8, 28.2.

### b) {3-[4-(2S-Hydroxymethyl-pyrrolidine-1-carbonyl)-2-methoxy-5-nitro-phenoxy]-propyl}-carbamic acid tert-butyl ester (36)

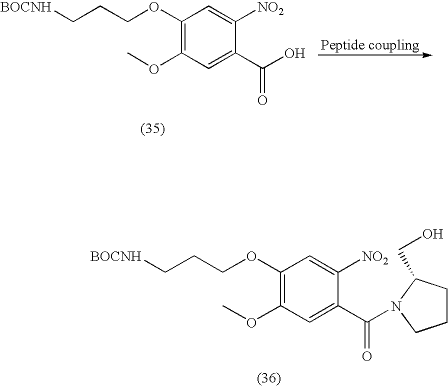

A solution of 35 (5.36 g, 14.4 mmol), EDCI (4.16 g, 21.7 mmol), and HOBt (3.32 g, 21.7 mmol) in anhydrous DMF (80 mL) were stirred at 30° C. for 3 h. Pyrrolidine-methanol (1.57 mL, 15.9 mmol) was added slowly at room temperature and the reaction was allowed to stir overnight. The following day the reaction mixture was diluted with EtOAc (250 mL) and washed with 10% aqueous citric acid (100 mL), water (2×200 mL), sat^{d} aqueous NaHCO₃ (200 mL), brine (200 mL), dried (MgSO₄), filtered and the solvent was removed in vacuo to yield 36 (6.1 g, 93%) which was pure as observed by TLC (EtOAc): ¹H NMR (400 MHz, CDCl₃) δ 7.68 (s, 1H), 6.81 (s, 1H), 5.24 (m, 1H), 4.38 (m, 2H), 4.18 (t, J=5.7 Hz), 3.99 (s, 3H), 3.95-3.80 (m, 2H), 3.38 (m, 2H), 3.18 (t, J=6.6 Hz, 2H), 2.19-1.74 (m, 6H), 1.45 (s, 9H); ¹³C NMR (100 MHz, CDCl₃) δ 156.0, 154.7, 148.4, 137.1, 128.0, 109.0, 108.1, 79.2, 68.4, 66.1, 61.5, 56.7, 49.5, 38.4, 29.1, 28.4, 24.4; Calculated for C₂₁H₃₁N₃O₈: C, 55.62; H, 6.89; N, 9.27. Found: C 55.45, H 7.03, N 9.05.

### c) [5-(3-tert-Butoxycarbonylamino-propoxy)-2-(2S-hydroxymethyl-pyrrolidine-1-carbonyl)-4-methoxy-phenyl]-carbamic acid allyl ester (37)

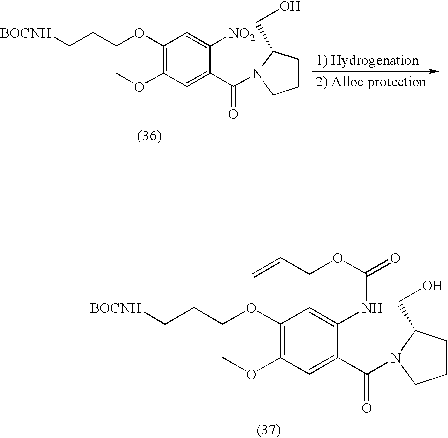

A slurry of 36 (6.1 g, 13.4 mmol) and 10% Pd/C (600 mg) in EtOAc (100 mL) was hydrogenated at 45 PSI using Parr apparatus. When hydrogen uptake ceased, the reaction was stopped and TLC analysis revealed completion of the reaction. The suspension was filtered through celite, the filtrate was dried (MgSO₄), filtered and the solvent removed in vacuo. The crude aniline was dissolved in anhydrous DCM (200 mL) in the presence of anhydrous pyridine (2.34 mL, 28.9 mmol) and stirred at 0° C. (acetone/ice bath). A solution of allyl chloroformate (1.28 mL, 12.1 mmol) in anhydrous DCM (60 mL) was added dropwise and the mixture was allowed to stir at room temperature overnight. The following day, the reaction mixture washed with 10% aqueous citric acid (100 mL), water (200 mL), sat^{d} aqueous NaHCO₃ (200 mL), brine (200 mL), dried (MgSO₄), filtered and the solvent removed in vacuo to yield 37 (5.85 g, 86%): ¹H NMR (400 MHz, CDCl₃) δ 8.76 (br s, 1H), 7.77 (s, 1H), 7.26 (s, 1H), 5.98 (m, 1H), 5.47 (m, 1H), 5.38-5.24 (dd, 2H), 4.63 (d, 2H), 4.45-4.20 (m, 2H), 4.15 (m, 2H), 3.85 (m, 4H), 3.80-3.45 (m, 3H), 3.35 (m, 2H), 2.18-1.60 (m, 6H), 1.46 (s, 9H); ¹³C NMR (100 MHz, CDCl₃) δ 170.8, 156.0, 153.6, 150.3, 144.0, 132.5, 131.8, 118.1, 111.0, 105.5, 78.9, 68.6, 68.0, 65.8, 61.0, 56.3, 38.8, 31.5, 29.1, 28.5, 28.3, 22.6; IR (CHCl₃) 3398, 2973, 1711, 1597, 1523, 1457, 1407, 1328, 1228, 1204, 1172, 1117, 1052, 917, 730 cm⁻¹; [α]²⁵_{D}=−80° (c=0.3, CHCl₃).

### d) (11S, 11 as)-8-(3-tert-Butoxycarbonylamino-propoxy)-11-hydroxy-7-methoxy-1,2,3,10,11,11a-hexahydro-5H-pyrrolo[2,1-c][1,4]benzodiazepine-10 carboxylic acid allyl ester (38)

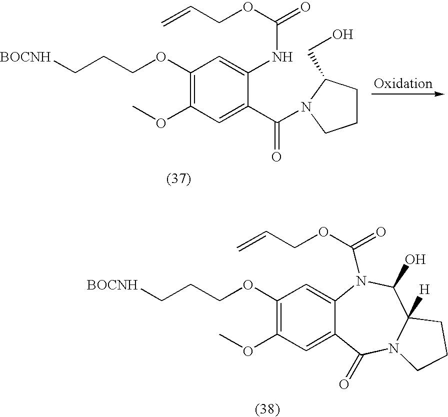

A solution of 37 (5.85 g, 11.5 mmol), diacetoxy-iodobenzene (4.1 g, 12.7 mmol) and TEMPO (182 mg, 1.15 mmol) in DCM (100 mL) was allowed to stir overnight. The following day, TLC (EtOAc) revealed completion of the reaction and the organic phase was washed with sat^{d} sodium metabisulphite, sat^{d} aqueous NaHCO₃, brine, dried (MgSO₄), filtered and the solvent was removed in vacuo. The residue was purified by column chromatography (gradient elution from 60:40 EtOAc/Hexane to 100% EtOAc). The pure fractions were evaporated in vacuo to yield the ring-closed product 38 as a white foam (4.19 g, 72%): ¹H NMR (400 MHz, CDCl₃) δ 7.24 (s, 1H), 6.67 (s, 1H), 5.80 (m, 1H), 5.64 (m, 1H), 5.47 (m, 1H), 5.13 (m, 2H), 4.68 (m, 1H), 4.45 (m, 1H), 4.15-3.98 (m, 2H), 3.92 (s, 3H), 3.78-3.67 (m, 1H), 3.60-3.42 (m, 2H), 3.34 (m, 2H), 2.18-1.95 (m, 6H); ¹³C NMR (100 MHz, CDCl₃) δ 167.0, 156.1, 156.0, 149.8, 148.5, 131.8, 128.3, 126.1, 118.0, 113.7, 110.5, 86.0, 79.0, 68.2, 66.7, 60.4, 60.0, 56.0, 46.4, 38.8, 29.1, 28.7, 28.5, 23.0; MS (ES⁺) m/z (relative intensity), 506 ([M+H]^{+.}, 100). 38 was optically pure as observed by chiral HPLC and is optically stable to treatment with TFA and extraction with conc^{d} NH₄OH.

### e) (11S, 11aS)-8-(3-Amino-propoxy)-11-hydroxy-7-methoxy-1,2,3,10,11,11aS-hexahydro-5H-pyrrolo[2,1-c][1,4]benzodiazepine-10-carboxylic acid allyl ester (10)

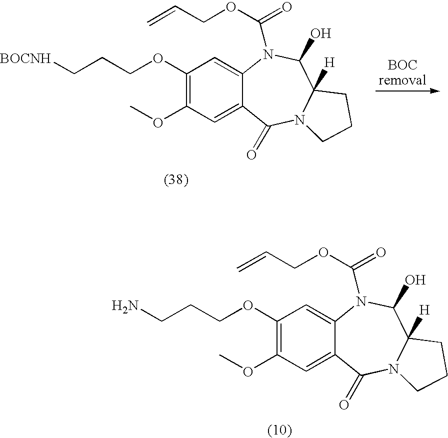

A mixture of 38 (1.1 g, 2.17 mmol), TFA (4 mL), DCM (6 mL) and water (0.5 mL) was stirred for 20 min at room temperature. After which time, TLC (CHCl₃) analysis revealed completion of the reaction. The mixture was diluted with ice and basified to pH 10 or greater with aqueous NH₄OH. The aqueous solution was then extracted with CHCl₃ (2×100 mL) and the combined organic layers washed with brine, dried (MgSO₄), filtered and the solvent was removed in vacuo to yield a white powder, which was pure as observed by TLC (90:10:1 v/v/v CHCl₃/MeOH/NH₄OH) (880 mg, 100%). This unstable amino capping unit 10 was used directly in the next coupling step without further purification.

### Example 4

### Synthesis of the Dimer (13) AT217

### a) Preparation of 18

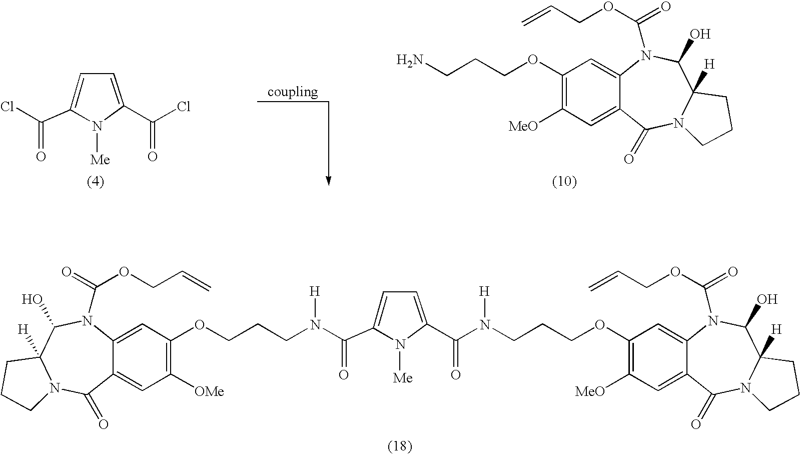

Oxalyl chloride (47 μL, 0.55 mmol) was added to a stirred solution of the diacid 3 (42 mg, 0.25 mmol) in anhydrous THF (5 mL) at room temperature. The mixture was then treated with a drop of DMF at which point vigorous effervescence occurred. The mixture was allowed to stir for 20 min at which point all effervescence had stopped. This acid chloride solution was then added dropwise to a stirred solution of the amide capping unit 10 (200 mg, 0.49 mmol) and TEA (151 μL, 1.08 mmol) in anhydrous THF (5 mL) at 0° C. under a nitrogen atmosphere. A white precipitate formed during the addition and the reaction mixture was allowed to warm to room temperature. After 4 h, the solvent was removed in vacuo and the residue was partitioned between CHCl₃ (30 mL) and aqueous 1N HCl (20 mL). The organic phase washed with sat^{d} aqueous NaHCO₃, brine, dried (MgSO₄), filtered and evaporated in vacuo to yield 18 (230 mg, quantitative yield) as a white powder which was used in the next step without further purification: ¹H NMR (400 MHz, DMSO-d₆) δ 8.26 (triplet, J=5.27 Hz, 2H), 7.10 (s, 2H), 6.79 (s, 2H), 6.68 (s, 2H), 6.51 (broad s, 2H), 5.92-5-68 (m, 2H), 5.58-5.39 (m, 2H), 5.17-4.93 (m, 4H), 4.74-4.32 (m, 4H), 4.14-3.93 (m, 7H), 3.83 (s, 6H), 3.50 (m, 2H), 3.44-3.23 (m, 8H), 2.14-1.81 (m, 12H); ¹³C NMR (100 MHz, DMSO-d₆) δ 165.0, 161.2, 154.4, 149.4, 147.0, 132.8, 129.0, 128.6, 116.9, 114.3, 110.7, 110.3, 85.4, 79.1, 66.6, 65.5, 64.9, 55.6, 45.0, 35.8, 33.9, 30.4, 28.8, 28.2, 25.1, 22.7, 15.1; MS (ES+) m/z (relative intensity) 944.5 ([M+H]^{+.}, 35), 926.5 (40), 908.6 (100); IR (CHCl₃) 3320, 2928, 2246, 1709, 1627, 1535, 1513, 1464, 1435, 1409, 1312, 1277, 1218, 1134, 1104, 1054, 1014, 913, 871, 770, 731, 646 cm⁻¹.

### b) Preparation of Dimer 13 (AT-217)

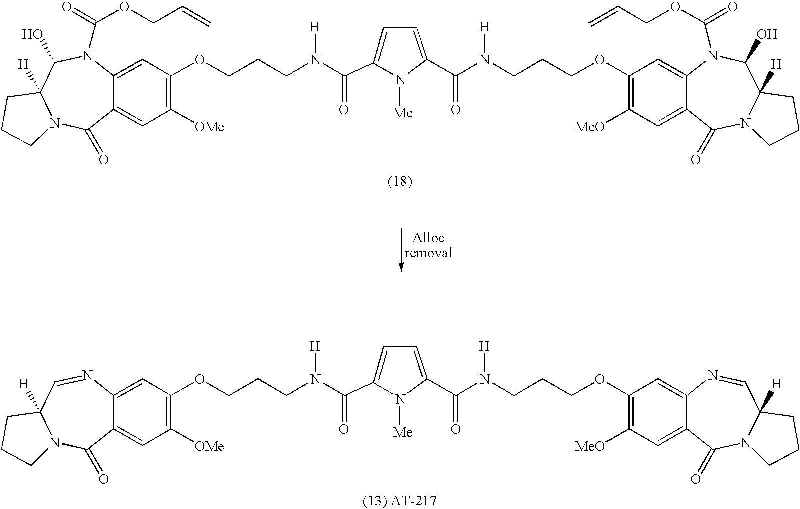

Tetrakis(triphenylphosphine)palladium (5 mg, 4 μmol) and pyrrolidine (37 μL, 0.44 mmol) were added to a stirred solution of 18 (190 mg, 0.2 mmol) and PPh₃ (6 mg, 0.02 mmol) in dry DCM under a nitrogen atmosphere. After 30 s, the formation of a white precipitate was observed. The reaction was allowed to stir for 10 mins at which point TLC showed reaction completion. The solvent was removed in vacuo to yield a white residue which was purified by flash chromatography using gradient elution (from 2:98 to 5:95 v/v MeOH/CHCl₃). Evaporation of the pure fractions yielded 13 (AT-217) (120 mg, 81%) as a white solid: ¹H NMR (400 MHz, CDCl₃) δ 7.68 (d, J=4.07 Hz, 2H), 7.51 (s, 2H), 6.96 (br s, 2H), 6.82 (s; 2H), 6.56 (s, 2H), 4.33-4.10 (m, 7H), 3.90-3.76 (m, 8H), 3.76-3.50 (m, 8H), 2.42-2.25 (m, 4H), 2.23-1.95 (m, 8H); ¹³C NMR (100 MHz, CDCl₃) δ 164.5, 162.6, 161.8, 150.3, 147.5, 140.6, 130.7, 120.6, 111.5, 110.3, 68.8, 55.9, 53.7, 46.7, 38.1, 34.4, 29.6, 28.7, 24.2; MS (ES+) m/z (relative intensity) 740 ([M+H]^{+.}, 100); IR (CHCl₃) 3320, 2951, 2237, 1656, 1622, 1600, 1534, 1505, 1463, 1432, 1383, 1340, 1262, 1216, 1092, 1019, 914, 874 cm⁻¹.

### Example 5

### Synthesis of Dimer 14 (AT-234)

### a) Preparation of 20

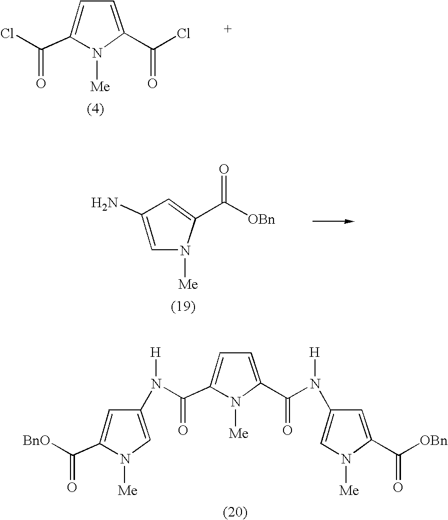

Oxalyl chloride (227 μL, 2.6 mmol) was added to a stirred solution of the diacid (3) (200 mg, 1.18 mmol) in anhydrous THF (5 mL) at room temperature. The mixture was then treated with a drop of DMF at which point vigorous effervescence occurred. The mixture was allowed to stir until all effervescence had stopped. This acid chloride solution was then added dropwise to a stirred suspension of the hydrochloride salt of 19 (631 mg, 2.36 mmol, J. Med. Chem., 26, 1983, 1042-49) and TEA (1.6 mL, 11.5 mmol) in anhydrous THF (10 mL) at 0° C. under a nitrogen atmosphere. The resulting white slurry was allowed to warm to room temperature and was stirred for 4 h. The solvent was then removed in vacuo and the residue was partitioned between CHCl₃ (100 mL) and aqueous 1N HCl (50 mL). The organic phase washed with water, sat^{d} aqueous NaHCO₃, brine, dried (MgSO₄), filtered and evaporated in vacuo to give a white powder, which was shown to contain two components by TLC (EtOAc/Hexane). The major component (lower spot) was isolated by recrystallisation (CHCl₃/diethyl ether) to provide 20 (160 mg, 23%): ¹H NMR (400 MHz, DMSO-d₆) δ 10.14 (s, 2H), 7.51 (d, J=1.82 Hz, 2H), 7.47-7.30 (m, 10H), 6.97 (d, J=1.90 Hz, 2H), 6.86 (s, 2H), 5.25 (s, 4H), 4.11 (s, 3H), 3.86 (s, 6H); ¹³C NMR (100 MHz, DMSO-d₆) δ 160.0, 158.1, 136.5, 130.1, 128.4, 127.9, 127.7, 122.6, 121.1, 118.6, 111.4, 108.7, 79.1, 64.9, 54.8, 36.2, 34.0; MS (ES⁺) m/z (relative intensity) 616 ([M+Na]^{+.}, 60), 594.2 ([M+H]^{+.}, 40), 353 (100); IR (CHCl₃) 3266, 3121, 2953, 1710, 1638, 1585, 1545, 1438, 1390, 1333, 1258, 1194, 1150, 1109, 1109, 1086, 1029, 1005, 895, 873, 808, 781, 744, 694, 630, 609 cm¹.

### b) Preparation of 22

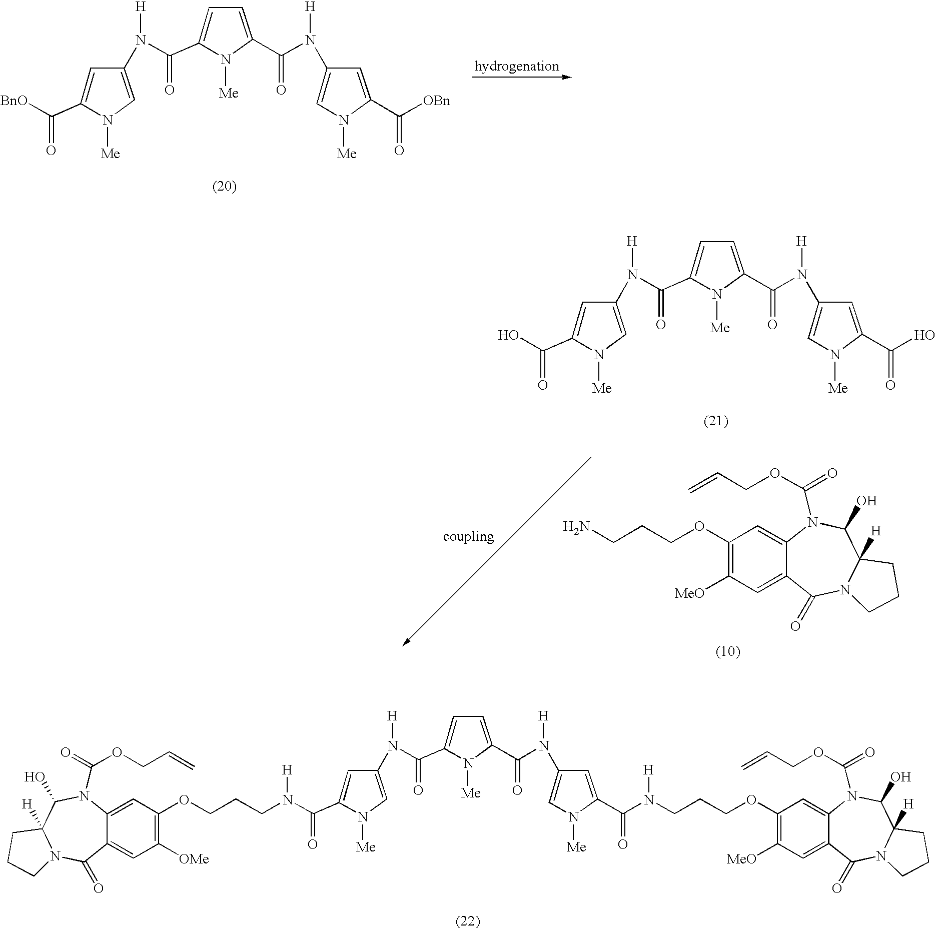

A slurry of 20 (150 mg, 0.25 mmol) and 10% Pd/C (30 mg) in anhydrous DMF (5 mL) was hydrogenated at 30 PSI using Parr apparatus for 6 h at which point TLC showed reaction completion. The suspension was filtered through celite directly into a round bottomed flask. The celite was quickly rinsed with anhydrous DMF (3 mL) and the combined filtrate was treated with HOBt (85 mg, 0.55 mmol) and EDCI (106 mg, 0.55 mmol) which resulted in the formation of a clear yellow solution that was stirred overnight. A sample of 10 (205 mg, 0.50 mmol) was then added and the solution was allowed to stir for 24 h. The solution was then partitioned between CHCl₃ (100 mL) and 10% aqueous citric acid (100 mL) and the organic phase washed with sat^{d} aqueous NaHCO₃, brine, dried (MgSO₄), filtered and the solvent was removed in vacuo. The residue was purified by column chromatography (3:97 to 4:96 v/v MeOH/CHCl₃) to yield 22 (180 mg, 60%): ¹H NMR (400 MHz, DMSO-d₆) δ 10.10 (s, 2H), 8.13 (t, J=4.94 Hz, 2H), 7.22 (d, J=1.10 Hz, 2H), 7.10 (s, 2H), 6.90 (d, J=1.16 Hz, 2H), 6.84 (s, 2H), 6.79 (s, 2H), 6.53 (broad s, 2H), 6.06-5.71 (m, 2H), 5.58-5.39 (m, 2H), 5.17-4.94 (m, 4H), 4.74-4.32 (m, 4H), 4.12 (s, 3H), 4.09-3.95 (m, 4H), 3.83 (m, 12H), 3.50 (m, 2H), 3.44-3.23 (m, 8H), 2.14-1.77 (m, 12H); ¹³C NMR (100 MHz, DMSO-d₆) δ 166.0, 161.3, 158.1, 154.4, 149.5, 148.0, 132.8, 130.2, 123.0, 121.7, 118.0, 116.9, 114.3, 111.2, 110.3, 104.2, 85.3, 79.1, 66.6, 60.5, 55.6, 45.9, 36.0, 35.60, 34.0, 29.0, 28.2, 22.7; MS (ES⁺) m/z (relative intensity) 1188 ([M+H]+., 100); IR (CHCl₃) 3320, 2928, 2246, 1709, 1627, 1535, 1513, 1464, 1435, 1409, 1312, 1277, 1218, 1134, 1104, 1054, 1014, 913, 871, 770, 731, 646 cm⁻¹.

### c) Preparation of Dimer 14 (AT-234)

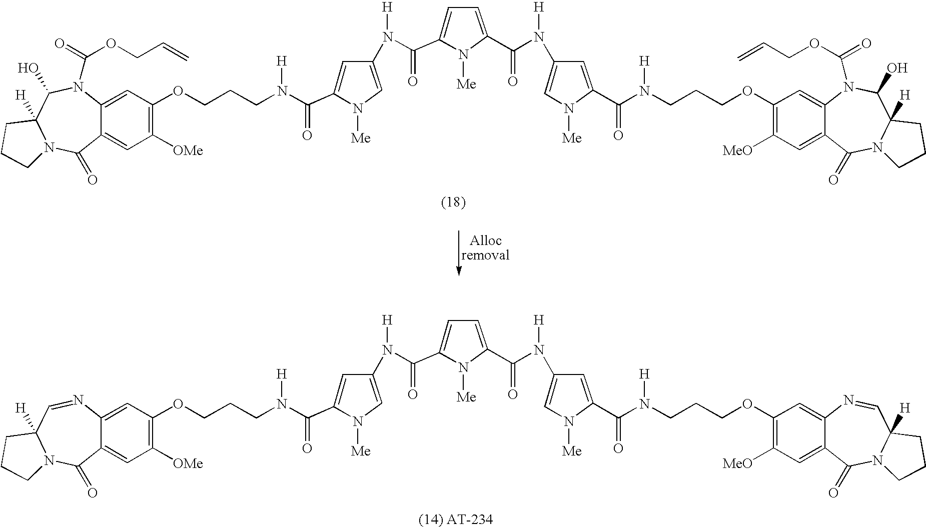

A sample of 22 (150 mg, 0.126 mmol) was dissolved in an anhydrous mixture of CHCl₃ (10 mL) and acetonitrile (10 mL) under a nitrogen atmosphere. PPh₃ (3 mg, 0.01 mmol), Pd(PPh₃)₄ (3 mg, 2.5 μmol), and pyrrolidine (22 μL, 0.26 mmol) were added simultaneously. Analysis by TLC showed reaction completion after 1 h. The solvent was removed in vacuo to yield a white residue which was purified by flash chromatography (gradient elution from 4:96 to 7:93 v/v MeOH/CHCl₃). Evaporation of the pure fractions yielded 14 (AT-234) (110 mg, 88%) as a white solid: ¹H NMR (400 MHz, DMSO-d₆) (diimine only) δ 10.1 (s, 2H), 8.12 (m, 2H), 7.78 (d, J=4.4 Hz, 2H), 7.35 (s, 2H), 7.23 (s, 2H), 6.89 (s, 2H), 6.84 (s, 4H), 4.12 (s, 3H), 4.22-3.92 (m, 4H), 3.84 (2s, 12H), 3.65-3.56 (m, 2H), 3.40-3.20 (m, 8H), 2.37-2.17 (m, 4H), 2.07-1.85 (m, 8H); ¹³C NMR (100 MHz, DMSO-d₆) δ 164.2, 163.3, 161.3, 158.1, 150.3, 146.9, 140.5, 130.2, 123.1, 121.7, 119.7, 118.0, 111.2, 110.0, 104.2, 66.4, 55.6, 53.4, 48.5, 46.3, 35.9, 35.6, 34.0, 23.6; MS (ES⁺) m/z (relative intensity) 984 ([M+H]^{+.}, 100); IR (CHCl₃) 3316, 2952, 1632, 1602, 1530, 1436, 1387, 1264, 1217, 1199, 1090, 1066, 1010, 872, 664 cm⁻¹.

### Example 6

### Synthesis of Acid Capping Unit 12

### a) (11S, 11aS)-7-Methoxy-8-(3-methoxycarbonyl-propoxy)-11-(tetrahydro-pyran-2-yloxy)-1,2,3,10,11,11a-hexahydro-5H-pyrrolo[2,1-c][1,4]benzodiazepine-10-carboxylic acid allyl ester (41).

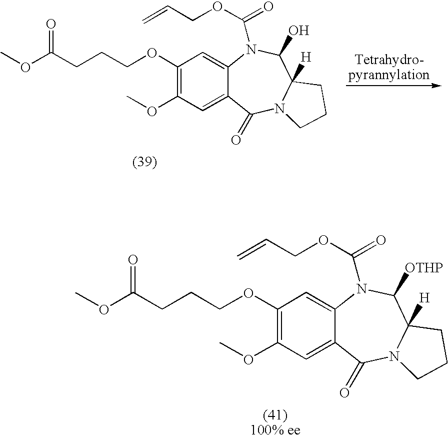

A solution of DHP (4.22 mL, 46.2 mmol) in EtOAc (30 mL) was stirred for 10 min in the presence of a crystal of p-TSA (catalytic quantity, 20 mg). A sample of 39 (2.0 g, 4.62 mmol) was then added in one portion to this solution and the mixture allowed to stir for 2 h. TLC analysis (EtOAc) revealed completion of reaction and the solution was diluted with EtOAc (70 mL) and washed with sat^{d} aqueous NaHCO₃ (50 mL), brine (50 mL), dried (MgSO₄), filtered and the solvent was removed in vacuo. The oily residue was further dried under strong vacuum to remove any remaining DHP to provide 41 in quantitative yield (2.38 g, 100%) which was used directly in the next step: ¹H NMR (CDCl₃, 400 MHz) as a 4:5 mixture of diastereoisomers: δ 7.24-7.21 (2s, 2H), 6.88-6.60 (2s, 2H), 5.89-5.73 (m, 4H), 5.15-5.04 (m, 6H), 4.96-4.81 (m, 2H), 4.68-4.35 (m, 4H), 4.12-3.98 (m, 4H), 3.98-3.83 (m, 8H), 3.74-3.63 (m, 8H), 3.60-3.40 (m, 8H), 2.56-2.50 (m, 4H), 2.23-1.93 (m, 12H), 1.92-1.68 (m, 10H), 1.66-1.48 (m, 20H); ¹³C NMR (CDCl₃, 100 MHz) δ 173.4, 167.3, 149.2, 132.0, 114.5, 100.0, 98.5, 94.7, 91.8, 68.0, 67.8, 66.3, 64.0, 63.6, 63.4, 62.9, 56.1, 51.6, 51.6, 46.4, 46.3, 31.1, 30.9, 30.7, 30.4, 30.3, 29.1, 25.4, 25.3, 25.3, 24.2, 20.0, 19.8, 19.7; MS (ES⁺) m/z (relative intensity) 533 ([M+H]^{+.}, 100).

### b) (11S, 11aS)-8-(3-Carboxy-propoxy)-7-methoxy-11-(tetrahydro-pyran-2-yloxy)-1,2,3,10,11,11a-hexahydro-5H-pyrrolo[2,1-c][1,4]benzodiazepine-10-carboxylic acid allyl ester (12)

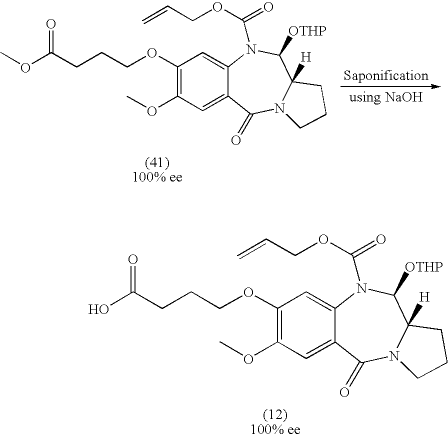

A solution of sodium hydroxide (340 mg, 8.5 mmol) in water (7 mL) was added to a stirred solution of 41 (2.2 g, 4.26 mmol) in MeOH (30 mL). The mixture was allowed to spin on the rotary evaporator at 70° C. for 15 min at atmospheric pressure, after which time the reaction was found to be complete by TLC analysis. The methanol was removed in vacuo and water (20 mL) was added. The aqueous solution was then acidified to pH<4 with 5% aqueous citric acid solution. The resulting precipitate was then extracted with EtOAc (100 mL) and the organic layer washed with brine (30 mL), dried (MgSO₄), filtered and the solvent was removed in vacuo. Diethylether (50 mL) was added to the residue and further evaporation in vacuo yielded 12 as a white foam (2.10 g, 98%): ¹H NMR (DMSO, 400 MHz) as a mixture of ⅘ of diastereoisomers δ 7.10 (2s, 2H), 6.90-6.84 (2s, 2H), 5.84-5.68 (m, 4H), 5.45-4.91 (m, 6H), 4.72-4.30 (m, 4H), 4.09-3.93 (m, 4H), 3.91-3.75 (m, 8H), 3.60-3.44 (m, 4H), 3.44-3.22 (m, 8H), 2.46-2.33 (m, 4H), 2.20-1.76 (m, 14H), 1.76-1.31 (m, 12H); ¹³C NMR (DMSO, 100 MHz) δ 174.0, 173.9, 171.9, 166.2, 166.1, 149.7, 148.4, 148.3, 132.7, 116.6, 114.4, 110.5, 110.3, 99.2, 67.6, 67.4, 65.7, 65.5, 62.9, 59.5, 55.7, 45.9, 30.6, 30.3, 29.9, 29.8, 28.4, 28.3, 24.9, 24.8, 24.0, 23.8, 22.9, 22.8; MS (ES⁺) m/z (relative intensity) 519 ([M+H]^{+.}, 100). 12 was shown to be optically pure at the C11a position by re-esterification (EDCI, HOBt, then MeOH), THP removal (AcOH/THF/H₂O) followed by chiral HPLC, as in Tercel et al., J. Med. Chem., 2003, 46, 2132-2151).

### Example 7

### Synthesis of Dimer 15 (AT-281)

### a) Preparation of 24

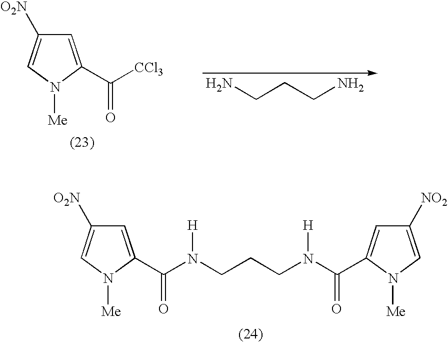

1-methyl-2-trichloroacetyl-4-nitropyrrole (23) (2 g, 7.36 mmol, J. Am. Chem. Soc., 118, 1996, 6141-46) and 1,3-diaminopropane (307 μL, 3.67 mmol) were dissolved in anhydrous THF (15 mL). The formation of a precipitate was observed after 5 min and the suspension was allowed to stir for 3 h. Following dilution with diethyl ether (60 mL), the precipitate was collected by filtration and dried in vacuo to yield 24 as an off-white fine powder (1.28 g, 92%): ¹H NMR (400 MHz, DMSO-d₆) δ 8.40 (t, J=5.4 Hz, 2H), 8.12 (d, J=1.51 Hz, 2H), 7.42 (d, J=1.88 Hz, 2H), 3.92 (s, 6H), 3.26 (q, J=6.49 Hz, 4H), 1.75 (p, J=6.88 Hz, 2H); ¹³C NMR (100 MHz, DMSO-d₆) δ 159.8, 133.7, 127.7, 126.4, 107.2, 37.3, 36.5, 28.8; MS (ES⁺) m/z (relative intensity) 379 ([M+H]^{+.}, 100); IR (CHCl₃) 3414, 3364, 3116, 2950, 1654, 550, 1530, 1501, 1418, 1346, 1311, 1264, 1222, 1209, 1139, 1111, 1075, 984, 950, 827, 849, 811, 747, 707 cm⁻¹.

### b) Preparation of 25

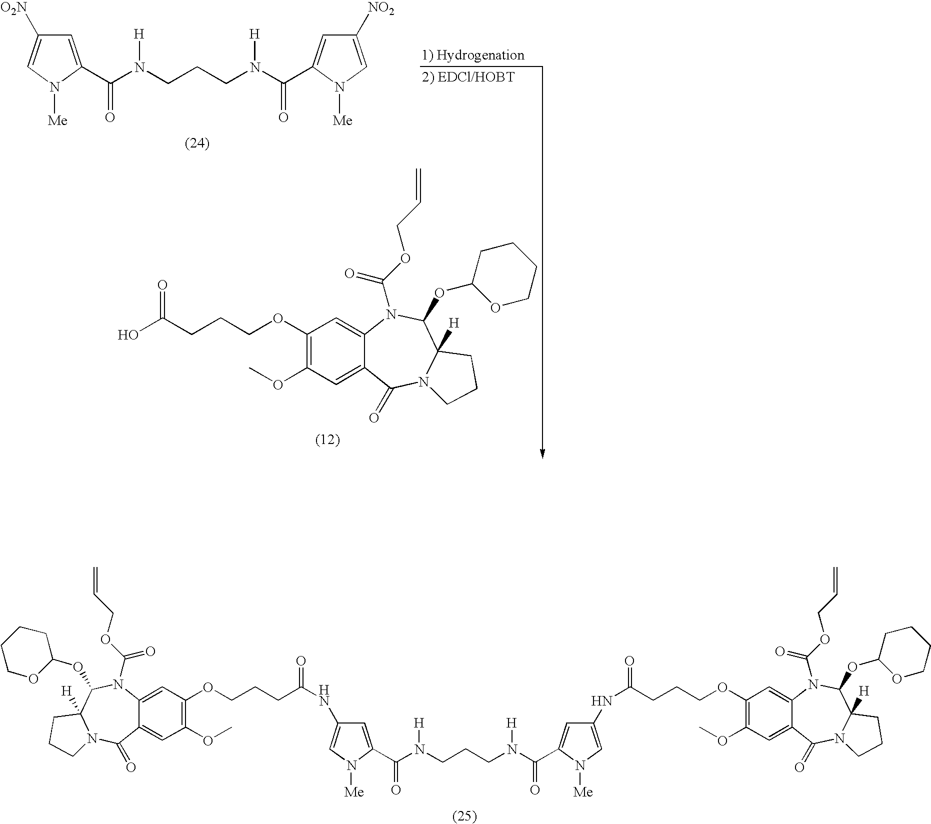

A slurry of 24 (100 mg, 0.264 mmol) and 10% Pd/C (40 mg) in anhydrous DMF (5 mL) was hydrogenated at 15 PSI using Parr apparatus. Completion of reaction was observed by TLC after 5 h. The hydrogenation suspension was filtered through celite directly into a round-bottomed flask containing a pre-prepared (stirred for 3 h) solution of 12 (265 mg, 0.528 mmol), EDCI (152 mg, 0.792 mmol) and HOBt (121 mg, 0.790 mmol) in anhydrous DMF (4 mL). The reaction mixture was stirred at 60° C. for a further 3 h and then overnight at room temperature. The following day the mixture was diluted with CHCl₃ (200 mL) and washed with water (200 mL), 10% aqueous citric acid (100 mL), sat^{d} aqueous NaHCO₃ (100 mL), brine (100 mL), dried (MgSO₄), filtered and the solvent was removed in vacuo. The residue was purified by flash chromatography (gradient elution from 1:99 to 3:97 v/v MeOH/CHCl₃) to yield 25 as an off-white solid (190 mg, 54.5%): ¹H NMR (400 MHz, DMSO-d₆) δ 9.82 (s, 2H), 8.00 (t, J=5.44 Hz, 2H), 7.12 (m, 4H), 6.93 (m, 1H), 6.83 (s, 1H), 6.67 (m, 2H), 5.79-5.68 (m, 4H), 5.88-5.58 (m, 6H), 4.70-4.31 (m, 4H), 4.06-3.96 (m, 4H), 3.79 (pseudo d, 14H), 3.58-3.46 (m, 4H), 3.35 (m, 4H), 3.20 (q, J=6.41 Hz, 4H), 2.40 (t, J=7 Hz, 4H), 2.19-1.77 (m, 12H), 1.65 (m, 6H), 1.55-1.28 (m, 8H); ¹³C NMR (100 MHz, DMSO-d₆) δ 168.7, 168.6, 166.2, 166.1, 161.2, 148.4, 148.2, 132.7, 122.9, 121.9, 117.5, 116.6, 114.4, 110.3, 103.2, 79.1, 68.0, 67.8, 65.5, 64.9, 62.8, 59.4, 55.7, 45.9, 36.1, 35.9, 31.7, 31.6, 30.6, 30.3, 29.7, 28.4, 28.3, 24.9, 24.7, 24.6, 24.4; MS (ES⁺) m/z (relative intensity) 1320 ([M+H]⁺, 100).

### c) Preparation of 15 (AT-281)

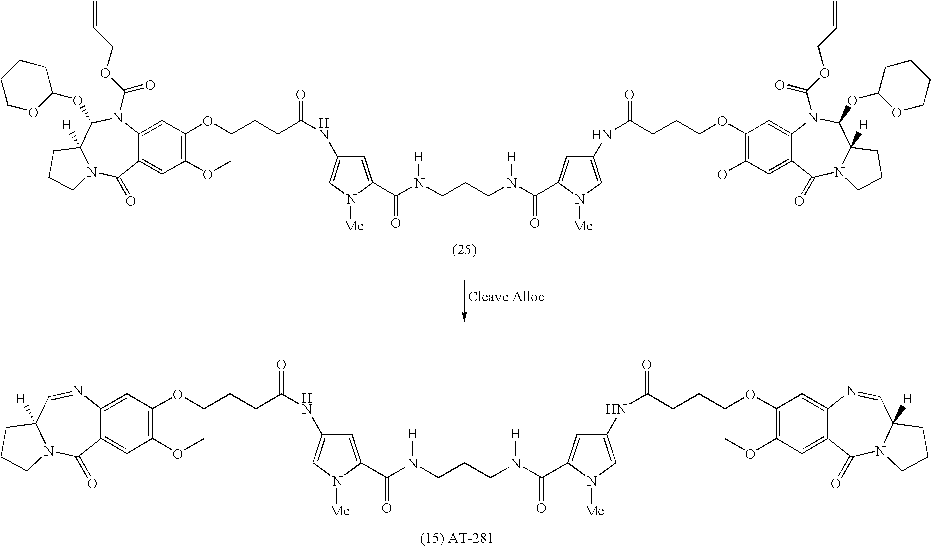

A stirred solution of 25 (138 mg, 0.104 mmol) in anhydrous CHCl₃ (5 mL) was treated with Pd(PPh₃)₄ (2.4 mg, 2.1 μmol) and pyrrolidine (19.2 μL, 0.246 mmol). After 1 h stirring at room temp reaction completion was observed by TLC analysis. The solvent was removed in vacuo and the residue purified by flash chromatography (gradient from 3:97 to 7:93 v/v MeOH/CHCl₃) to yield 15 (AT-281) as an off-white solid (99 mg, 90%). A sample was dissolved in deuterated DMSO and NMR data of the diimine form was recorded after at least 48 h standing at room temperature: ¹H NMR (400 MHz, DMSO-d₆) δ 9.83 (s, 2H), 8.01 (t, J=5.73 Hz, 2H), 7.79 (d, J=4.4 Hz, 2H), 7.35 (s, 2H), 7.12 (s, 2H), 6.83 (s, 2H), 6.69 (s, 2H), 4.20-3.99 (m, 4H), 3.82 (m, 12H), 3.75-3.58 (m, 4H), 3.47-3.40 (m, 2H), 3.22 (m, 4H), 2.43 (t, J=7.3 Hz, 4H), 2.33-2.15 (m, 4H), 2.12-1.94 (m, 8H), 1.67 (m, 2H); ¹³C NMR (100 MHz, DMSO-d₆) δ 168.7, 164.2, 163.3, 161.2, 150.2, 146.9, 140.6, 122.9, 121.9, 119.8, 117.5, 111.3, 110.1, 103.3, 79.1, 67.8, 56.0, 55.6, 53.4, 46.3, 36.1, 35.9, 31.8, 29.7, 28.8, 24.7, 23.6, 18.5; MS (ES⁺) m/z (relative intensity) 947 ([M+H]^{+.}, 100).

### Example 8

### Synthesis of Dimer 16 (AT-242)

### a) Preparation of 26

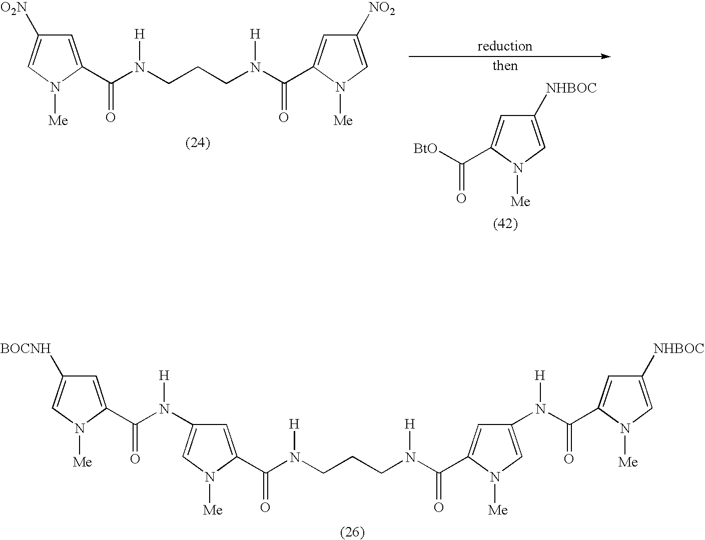

A slurry of 24 (200 mg, 0.53 mmol) and 10% Pd/C (50 mg) in anhydrous DMF (7 mL) was hydrogenated at 15 PSI using Parr apparatus. Completion of the reaction was observed by TLC after 6 h. The suspension was filtered through celite directly into a round-bottomed flask containing 42 (377 mg, 1.06 mmol, J. Am. Chem. Soc., 118, 1996, 6141-46). The reaction mixture was stirred at 60° C. for 1 h and then overnight at room temperature. The following day the mixture was extracted with chloroform (200 mL), washed with water (200 mL), 10% aqueous citric acid (100 mL), sat^{d} aqueous NaHCO₃ (100 mL), brine (100 mL), dried (MgSO₄), filtered and the solvent was removed in vacuo. The residue was purified by flash chromatography (3.5:96.5 v/v MeOH/CHCl₃) to yield 26 as an off-white solid (284 mg, 70%): ¹H NMR (400 MHz, DMSO-d₆) δ 9.83 (s, 2H), 9.09 (s, 2H), 8.03 (t, J=5.6 Hz, 2H), 7.19 (d, J=1.37 Hz, 2H), 6.89 (s, 4H), 6.84 (s, 2H), 3.82 (s, 12H), 3.24 (q, J=6.16 Hz, 4H), 1.71 (p, J=6.70 Hz, 2H), 1.47 (s, 18H); ¹³C NMR (100 MHz, DMSO-d₆) δ 161.3, 158.3, 152.8, 122.9, 122.8, 122.3, 122.1, 117.7, 117.0, 104.1, 103.7, 36.0, 35.9, 29.7, 28.2; MS (ES⁺) m/z (relative intensity) 763 ([M+H]^{+.}, 100); IR (CHCl₃) 3311, 2977, 1697, 1643, 1586, 1529, 1434, 1402, 1364, 1248, 1206, 1161, 1098, 1063, 997, 895, 804, 757, 664 cm⁻¹.

### b) Preparation of 27

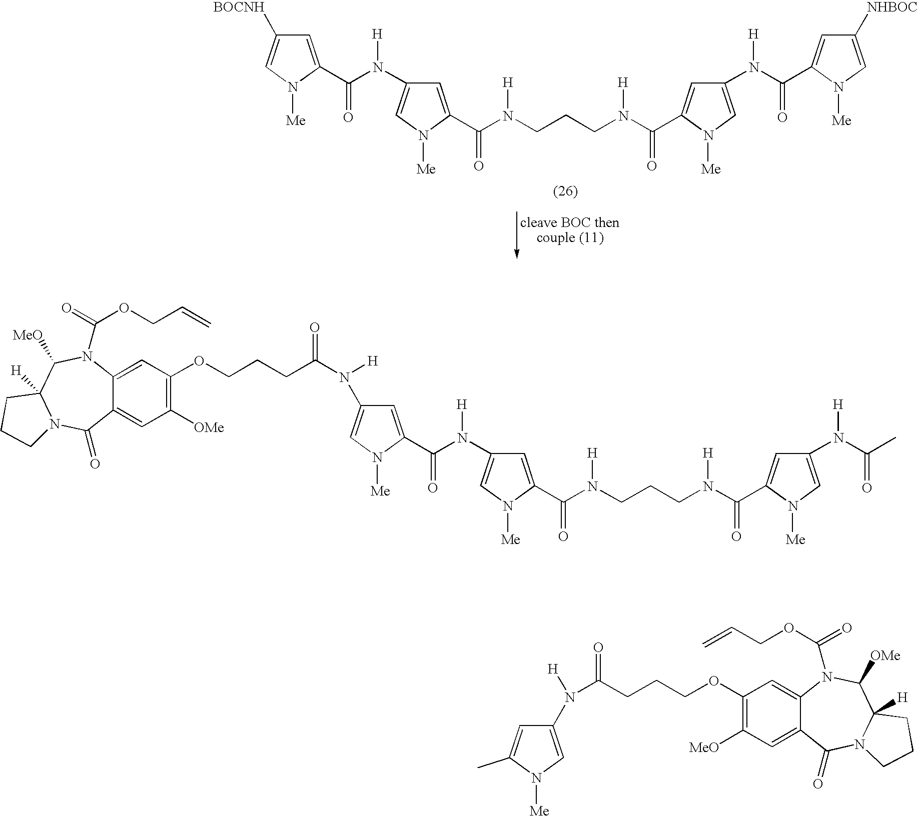

Finely ground 26 (200 mg, 0.262 mmol) was treated with 4N HCl in dioxane (10 mL) and the mixture vigorously stirred. After a short time, the formation of a white precipitate was observed. The mixture was allowed to stir for a further 45 min and the solvent was removed in vacuo. This salt was suspended in anhydrous THF (7 mL) in the presence of DIPEA (0.45 mL, 2.59 mmol) and treated dropwise with a pre-prepared (stirred for 10 min) mixture of 11 (235 mg, 0.532 mmol), oxalyl chloride (65 μL, 0.549 mmol) and DMF (1 drop) in anhydrous THF (8 mL). Further anhydrous DMF (5 mL) was then added and the suspension became a solution. After 1 h stirring, completion of the reaction was observed by TLC and the solvent was removed in vacuo. The residue was dissolved in CHCl₃, washed with water (200 mL), 10% aqueous citric acid (100 mL), sat^{d} aqueous NaHCO₃ (100 mL), brine (100 mL), dried (MgSO₄), filtered and the solvent was removed in vacuo. The residue was purified by flash chromatography (gradient elution from neat CHCl₃ to 4:96 v/v MeOH/) to give 27 as an off-white solid (217 mg, 58%): ¹H NMR (400 MHz, DMSO-d₆) δ 9.88-9.86 (m, 4H), 8.04 (t, J=5.6 Hz, 2H), 7.20-7.18 (m, 4H), 7.11 (s, 2H), 6.89 (m, 6H), 5.79 (m, 2H), 5.36 (d, J=9 Hz, 2H), 5.04 (m, 4H), 4.58-4.44 (m, 4H), 4.04 (m, 4H), 3.83 (s, 18H), 3.47 (s, 8H), 3.33 (m, 4H), 3.24 (q, J=6.10 Hz, 4H), 2.45 (t, J=7.15 Hz, 4H), 2.06 (m, 6H), 1.97-1.85 (m, 6H), 1.71 (p, J=6.56 Hz, 2H); ¹³C NMR (100 MHz, DMSO-d₆) δ 168.8, 166.1, 161.3, 158.3, 149.7, 148.3, 132.1, 128.4, 122.9, 122.7, 122.0, 122.0, 118.1, 117.8, 114.4, 110.4, 104.0, 103.8, 92.9, 79.1, 68.1, 55.6, 45.9, 36.0, 35.9, 31.7, 28.4, 22.8; MS (ES⁺) m/z (relative intensity) 1424 ([M+H]^{+.}, 60), 681 (100); IR (CHCl₃) 3311, 2977, 1697, 1643, 1586, 1529, 1434, 1402, 1364, 1248, 1206, 1161, 1098, 1063, 997, 895, 804, 757, 664 cm⁻¹.

### c) Preparation of Dimer 16 (AT-242)

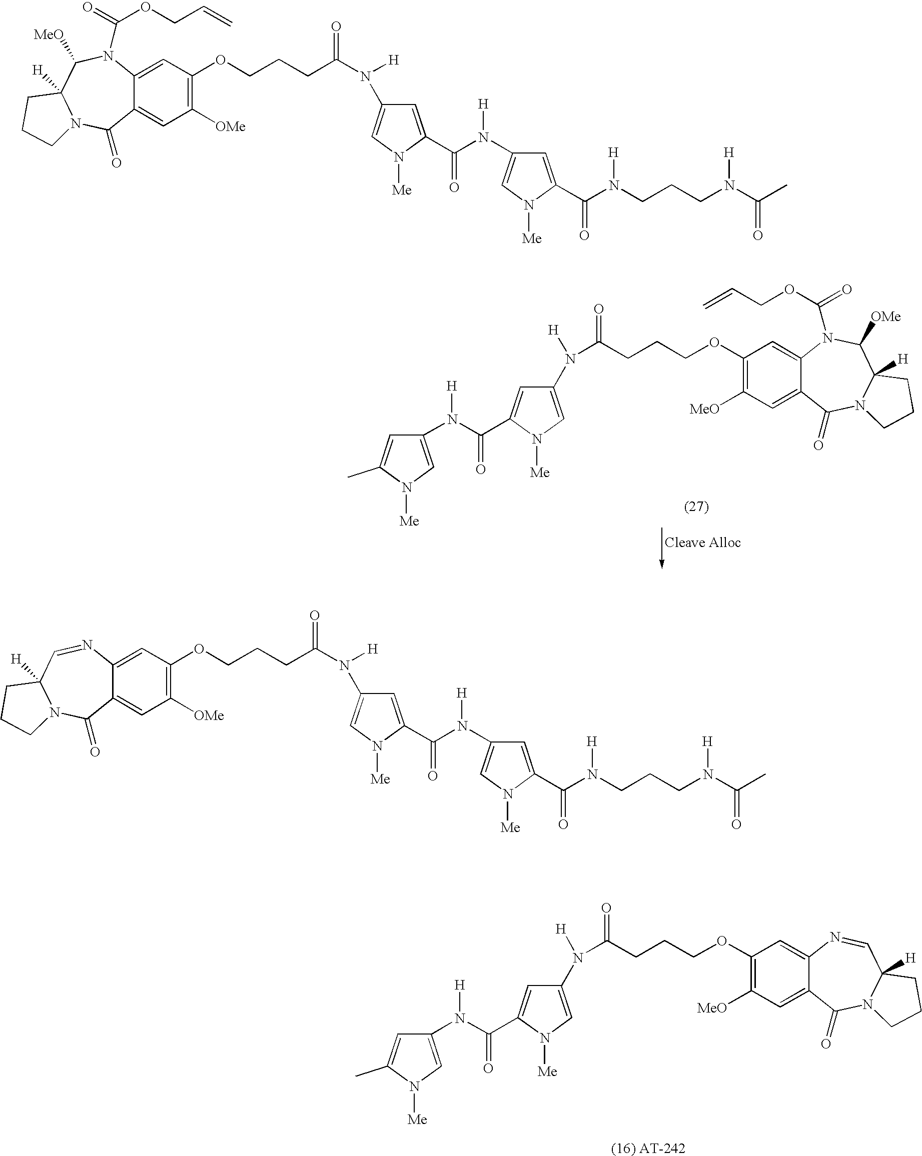

A stirred solution of 27 (167 mg, 0.117 mmol) in anhydrous CHCl₃ (5 mL) was treated with Pd(PPh₃)₄ (2.7 mg, 2.3 μmol) and pyrrolidine (20.6 μL, 0.246 mmol). After stirring at room temperature for 1 h reaction completion was observed by TLC. The solvent was removed in vacuo and the residue purified by flash chromatography (gradient elution from 4:96 to 8:92 v/v MeOH/CHCl₃) to yield 16 (AT-242) as an off-white solid (123 mg, 88%). A sample was dissolved in deuterated DMSO and NMR data of the diimine form was recorded after standing for at least 48 h: ¹H NMR (400 MHz, DMSO-d₆) (diimine only) δ 9.88 (m, 4H), 8.04 (t, J=5.4 Hz, 2H), 7.79 (d, J=4.4 Hz, 2H), 7.35 (s, 2H), 7.20-7.18 (m, 4H), 6.88 (s, 4H), 6.84 (s, 2H), 4.15-4.04 (m, 4H), 3.84 (m, 18H), 3.70-3.58 (m, 4H), 3.43-3.36 (m, 2H), 3.24 (m, 4H), 2.45 (t, J=7.3 Hz, 4H), 2.29-2.24 (m, 4H), 2.09-1.94 (m, 8H), 1.69 (m, 2H); ¹³C NMR (100 MHz, DMSO-d₆) δ 168.8, 164.2, 163.3, 161.3, 158.3, 150.2, 146.9, 140.5, 122.9, 122.7, 122.0, 121.9, 119.8, 118.1, 117.8, 111.2, 110.1, 104.0, 103.9, 67.8, 55.6, 53.4, 46.3, 36.0, 35.9, 31.9, 29.7, 28.8, 24.7, 23.6; MS (ES⁺) m/z (relative intensity) 1197 ([M+H]^{+.}, 100); [α]²⁵_{D}=+315° (c=0.45, CHCl₃); IR (CHCl₃) 3295, 2947, 1639, 1597, 1532, 1508, 1434, 1405, 1341, 1263, 1215, 1155, 1095, 1016, 752, 664 cm⁻¹.

### Example 9

### Synthesis of 17 (AT-288)

### a) Preparation of 30

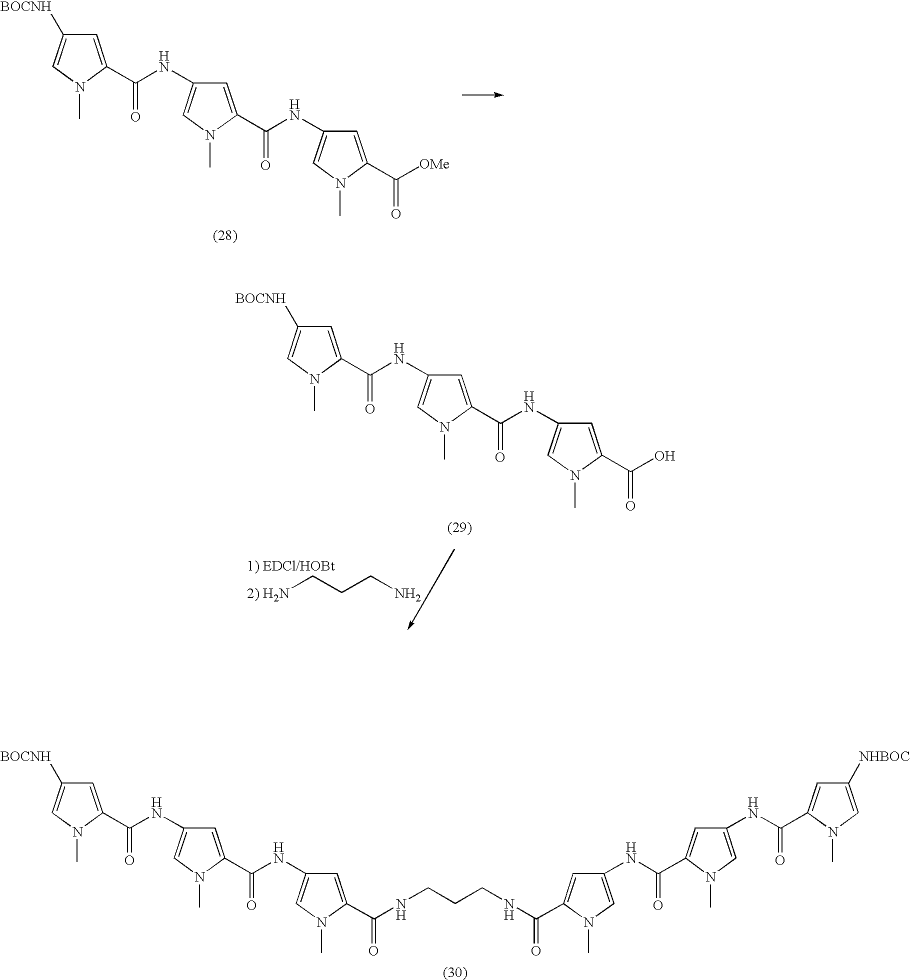

A solution of 29 (450 mg, 0.92 mmol, J. Am. Chem. Soc., 122, 2000, 6382-94) in anhydrous DMF (6 mL) was treated with EDCI (195 mg, 1.02 mmol) and HOBt (155.2 mg, 1.02 mmol) and stirred for 5 h. 1,3-diaminopropane (42.2 μl, 0.50 mmol) was then added and the resulting mixture was allowed to stir overnight. The following day, completion of reaction was observed by TLC and the mixture was diluted with CHCl₃ (100 mL), washed with 5% aqueous citric acid (50 mL), water (50 mL), sat^{d} aqueous NaHCO₃ (50 mL), brine (50 mL) and dried (MgSO₄). The mixture was filtered and solvent removed in vacuo to provide a yellow oil which was triturated with diethylether and the solvent was decanted off. The supernatant was removed and the residue was dried in vacuo to yield 30 as an off-white powder (400 mg, 86%): ¹H NMR (400 MHz, DMSO-d₆) δ 9.86 (m, 4H), 9.08 (s, 2H), 8.05 (t, J=5.61 Hz, 2H), 7.23 (m, 4H), 7.06 (s, 2H), 6.90 (m, 6H), 3.86 (m, 18H), 3.26 (q, J=6.35 Hz, 4H), 1.71 (m, 2H), 1.47 (s, 18H); ¹³C NMR (100 MHz, DMSO-d₆) δ 162.3, 161.3, 158.5, 158.4, 152.8, 122.9, 122.8, 122.7, 122.3, 122.2, 118.3, 117.8, 117.0, 104.7, 104.1, 103.8, 78.2, 64.9, 36.0, 36.0, 35.9, 35.7, 30.7, 29.7, 28.2, 15.1; MS (ES⁺) m/z (relative intensity) 1007 ([M+H]^{+.}, 7), 833 (100).

### b) Preparation of 31

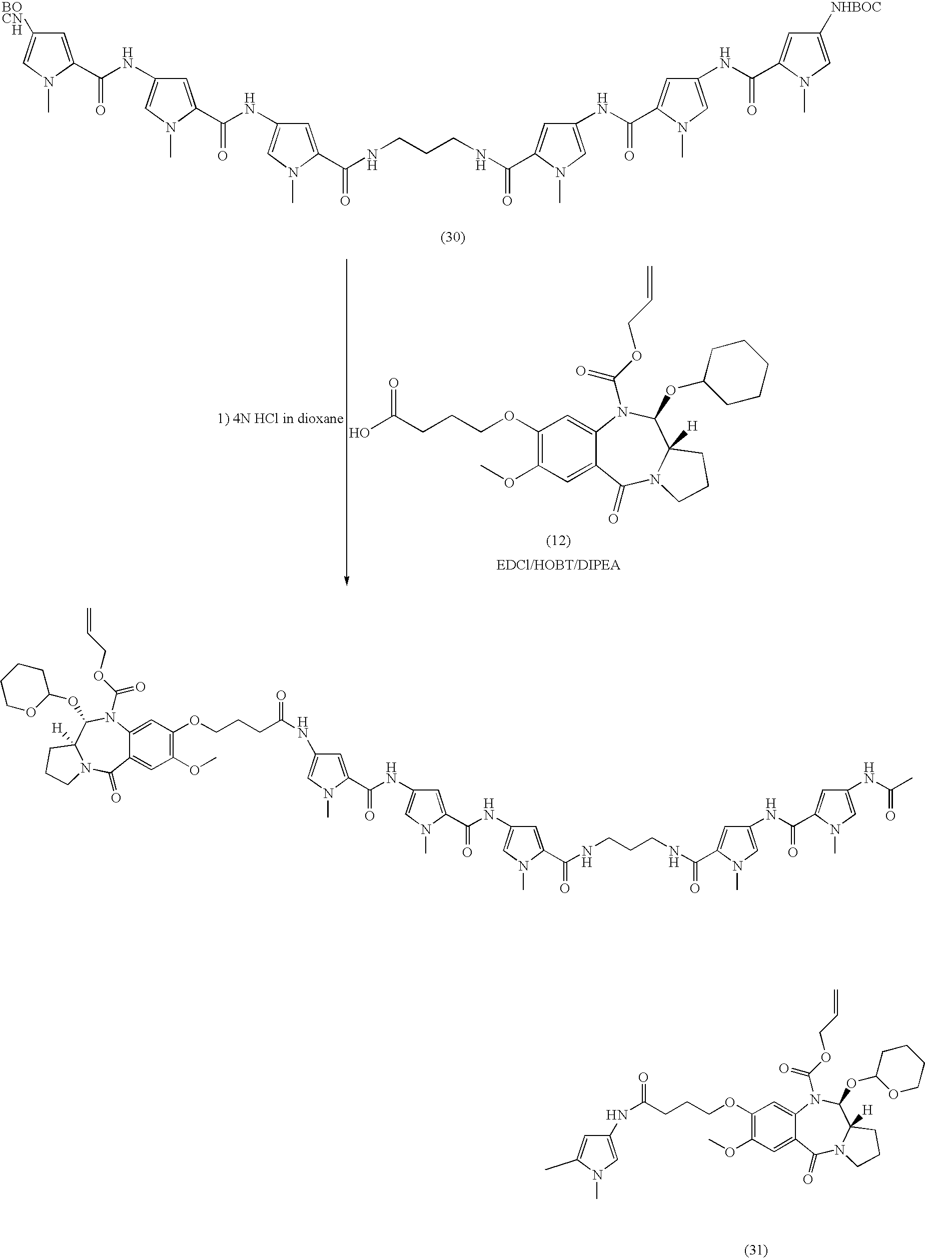

A solution of acid capping unit 12 (300 mg, 0.597 mmol) in anhydrous DMF (5 mL) was treated with EDCI (126 mg, 0.656 mmol) and HOBt (100 mg, 0.653 mmol) and allowed to stir for 3 h. Finely ground 30 (300 mg, 0.298 mmol) was suspended in 4N HCl-dioxane (10 mL). Following stirring for 1 h the mixture was subjected to ultrasound for 3 min and the volatiles were removed in vacuo to provide a grey solid. This was then dissolved in anhydrous DMF (5 mL) and DIPEA (0.5 mL, 2.87 mmol) and treated dropwise with the aforementioned activated acid solution. The resulting mixture was stirred at 60° C. for 1 h and then overnight at room temperature. The following day, completion of reaction was observed by TLC and the mixture was diluted with CHCl₃ (100 mL), washed with 5% aqueous citric acid (50 mL), water (50 mL), sat^{d} aqueous NaHCO₃ (50 mL), brine (50 mL), dried (MgSO₄), filtered and the solvent removed in vacuo to provide a yellow oil. The residue was triturated with diethylether and decanted. The supernatant was removed and the residue was dried in vacuo to furnish a tan solid which was purified by flash chromatography (gradient elution from neat CHCl₃ to 4:96 v/v MeOH/CHCl₃) to yield pure 31 (250 mg, 46%): ¹H NMR (400 MHz, DMSO-d₆) δ 9.89 (m, 6H), 8.06 (t, J=5.55 Hz, 2H), 7.21 (m, 6H), 7.10 (m, 4H), 6.89 (m, 6H), 5.75 (m, 4H), 5.18-4.92 (m, 6H), 4.69-4.32 (m, 4H), 4.01 (m, 4H), 3.86 (m, 26H), 3.50 (m, 4H), 3.40 (m, 4H), 3.26 (q, J=6.34 Hz, 4H), 2.44 (t, J=7.04 Hz, 4H), 2.18-1.77 (m, 12H), 1.76-1.56 (m, 6H), 1.56-1.30 (m, 8H); ¹³C NMR (100 MHz, DMSO-d₆) δ 168.8, 166.2, 161.3, 158.4, 158.4, 148.4, 148.2, 132.7, 122.9, 122.7, 122.7, 122.2, 122.1, 122.0, 118.4, 118.1, 117.8, 114.4, 104.7, 104.1, 103.8, 79.1, 68.0, 64.9, 55.7, 45.9, 40.1, 36.0, 35.9, 31.8, 30.3, 29.7, 28.4, 28.3, 24.9, 24.7, 24.4, 22.9, 19.3, 15.1.

### c) Preparation of Dimer 17 (AT-288)

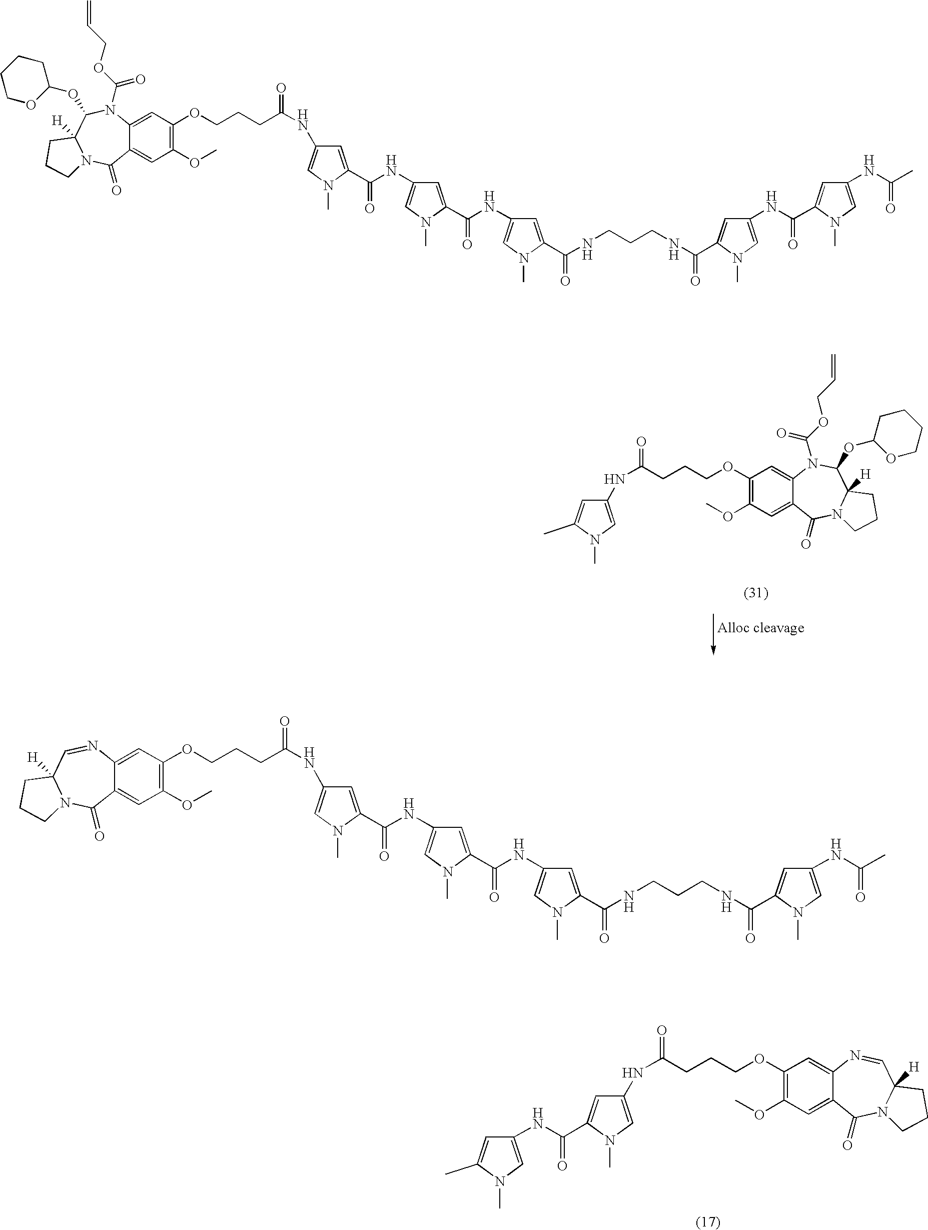

A solution of 31 (200 mg, 0.111 mmol) in anhydrous CHCl₃ (6 mL) was treated with Pd(PPh₃)₄ (2.5 mg, 2.1 μmol) and pyrrolidine (19.5 μL, 0.233 mmol). After stirring for 2 h completion of reaction was observed by TLC. The solvent was removed in vacuo and the residue purified by flash chromatography (gradient elution from 4:96 to 9:91 v/v MeOH/CHCl₃) to yield 17 (AT-288) as an off-white solid (123 mg, 77%). A sample was dissolved in deuterated DMSO and NMR data of the diimine form was recorded after standing for at least 48 h: ¹H NMR (400 MHz, DMSO-d₆) (diimine only) δ 9.89 (m, 6H), 8.04 (pseudo triplet, 2H), 7.79 (d, J=4.4 Hz, 2H), 7.35 (s, 2H), 7.25 (s, 2H), 7.21 (s, 2H), 7.18 (s, 2H), 7.05 (s, 2H), 6.90 (s, 4H), 6.84 (s, 2H), 4.15-4.04 (m, 4H), 3.84 (m, 24H), 3.72-3.58 (m, 4H), 3.41-3.37 (m, 2H), 3.24 (m, 4H), 2.45 (t, J=7.3 Hz, 4H), 2.30-2.24 (m, 4H, C1), 2.12-1.90 (m, 8H), 1.70 (m, 2H); ¹³C NMR (100 MHz, DMSO) δ 168.8, 164.2, 163.3, 161.3, 158.4, 158.4, 151.2, 146.9, 140.6, 122.9, 122.7, 122.7, 122.2, 122.1, 122.0, 119.8, 118.4, 118.1, 117.8, 111.3, 110.2, 104.7, 104.1, 104.0, 79.1, 67.8, 55.6, 53.4, 48.6, 46.3, 36.0, 35.9, 31.9, 28.8, 24.7, 23.6, 22.6; MS (ES⁺) m/z (relative intensity) 1435 ([M+H]^{+.}, 35), 718 (100).

### Example 10

### Synthesis of Acid Capping Unit (11)

### a) (11S, 11aS)-7,11-Dimethoxy-8-(3-methoxycarbonyl-propoxy)-1,2,3,10,11,11a-hexahydro-5H-pyrrolo[2,1-c][1,4]benzodiazepine-10-carboxylic acid allyl ester (40)

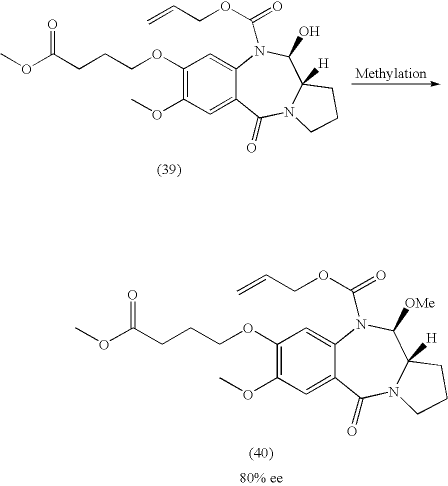

A solution of 39 (1.2 g, 2.77 mmol) in anhydrous DCM (50 mL) was treated with MeI (1.72 mL, 27.6 mmol) and silver (I) oxide (2.5 g, 10.8 mmol) and the suspension was heated at reflux for 24 h. The reaction was then found to be complete as judged by TLC (EtOAc). The suspension was filtered through celite and the volatiles were removed in vacuo (caution: MeI is carcinogenic, evaporate in a fumehood) to yield the methyl ether 40 (1.27 g, 99%): Chiral HPLC revealed 40 to be a mixture of two enantiomers in a 90:10 ratio (90% of the C11aS isomer, 10% of the C11aR isomer); ¹H NMR (CDCl₃, 400 MHz) δ 7.22 (s, 1H), 6.65 (s, 1H), 5.86-5.65 (m, 1H), 5.43 (d, J=9.09 Hz, 1H), 5.18-4.94 (m, 2H), 4.71-4.32 (m, 2H), 4.07 (t, J=6.21 Hz, 2H), 3.89 (s, 3H), 3.66 (m, 4H), 3.47 (m, 4H), 3.40 (m, 1H), 2.54 (t, J=7.19 Hz, 2H), 2.14 (p, J=6.57 Hz, 2H), 2.09-1.91 (m, 4H); ¹³C NMR (CDCl₃, 100 MHz) δ 173.4, 167.2, 156.2, 150.1, 149.1, 132.0, 128.6, 126.8, 117.2, 114.4, 110.9, 93.3, 68.0, 66.4, 60.1, 56.5, 56.1, 51.6, 46.3, 30.3, 29.0, 24.2, 23.2; MS (ES⁺) m/z (relative intensity) 463 ([M+H]^{+.}, 100).

### b) (11S, 11aS)-8-(3-Carboxy-propoxy)-7,11-dimethoxy-1,2,3,10,11,11a-hexahydro-5H-pyrrolo[2,1-c][1,4]benzodiazepine-10-carboxylic acid allyl ester (11)

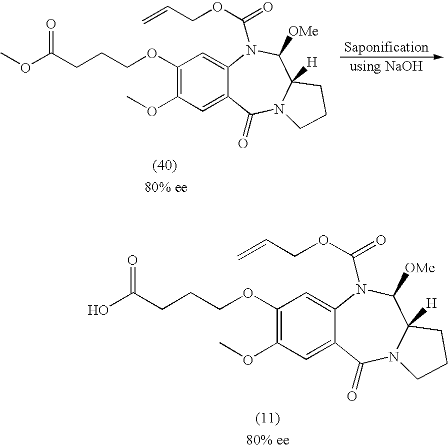

A solution of sodium hydroxide (207 mg, 5.19 mmol) in water (5 mL) was added to a stirred solution of 40 (1.2 g, 2.59 mmol) in MeOH (20 mL). The mixture was allowed to spin on a rotary evaporator at 70° C. for 15 min at atmospheric pressure after which time the reaction was found to be complete as judged by TLC (90:10:1 v/v EtOAc/MeOH/AcOH,). The methanol was removed in vacuo and water (20 mL) was added. The aqueous solution was acidified to pH<3 with 1N aqueous HCl and extracted with EtOAc (2×50 mL). The organic layers were combined, washed with brine (30 mL), dried (MgSO₄), filtered and the solvent was removed in vacuo. Diethylether (50 mL) was added to the residue and further evaporation in vacuo yielded 11 as a white foam (1.15 g, 99%): ¹H NMR (DMSO-d₆, 400 MHz) δ 7.24 (s, 1H), 6.65 (s, 1H), 5.85-5.66 (m, 1H), 5.43 (d, J=9.01 Hz, 1H), 5.17-4.95 (m, 2H), 4.68-4.34 (m, 2H), 4.09 (t, J=6.95 Hz, 2H), 3.89 (s, 3H), 3.74-3.61 (m, 1H), 3.59-3.46 (m, 4H), 3.46-3.35 (m, 1H), 2.59 (t, J=7.15 Hz, 2H), 2.16 (quint, J=6.58 Hz, 2H), 2.10-1.89 (m, 4H); ¹³C NMR (DMSO, 100 MHz) δ 177.8, 176.3, 167.4, 156.1, 150.2, 149.2, 131.9, 128.6, 126.7, 117.3, 114.4, 110.9, 93.4, 67.9, 66.5, 60.4, 60.2, 56.5, 56.1, 46.4, 30.2, 28.9, 24.0, 23.2, 21.0; MS (ES⁺) m/z (relative intensity) 449 ([M+H]^{+.}, 100); IR (CHCl₃) 2942, 1712, 1603, 1516, 1467, 1436, 1408, 1314, 1275, 1205, 1111, 1087, 1040, 972, 918, 767, 731 cm⁻¹.

### Example 11

### Synthesis of Dimer 18 (AT-235)

### a) Preparation of 32

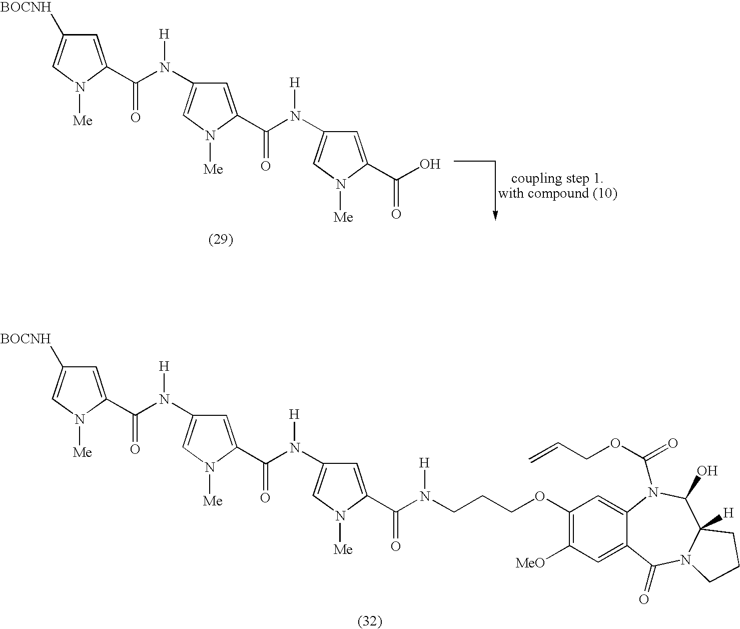

A solution of 29 (197 mg, 0.404 mmol) in anhydrous DMF (5 mL) was treated with EDCI (81 mg, 0.422 mmol) and HOBt (65 mg, 0.425 mmol) and was stirred for 7 h. A solution of the amine capping unit (10) (164 mg, 0.404 mmol) in anhydrous DMF (5 mL) was then added and the resulting mixture was allowed to stir overnight at room temperature. The mixture was diluted with CHCL₃ (200 mL), washed with 5% aqueous citric acid (100 mL), water (100 mL), sat^{d} aqueous NaHCO₃ (100 mL), brine (100 mL), dried (MgSO₄), filtered and the solvents were removed in vacuo to provide a yellow oil Purification by flash chromatography (1:99 v/v MeOH/CHCl₃) yielded 32 (250 mg, 71%): ¹H NMR (400 MHz, DMSO-d₆) δ 9.88 (m, 2H), 9.09 (s, 1H), 8.09 (t, J=5.24 Hz), 7.20 (m, 2H), 7.10 (s, 1H), 7.05 (d, J=1.35 Hz, 1H), 6.90 (s, 1H), 6.85 (s, 1H), 6.79 (s, 1H), 6.53 (br s, 1H), 5.89-5.71 (m, 1H), 5.48 (m, 1H), 5.16-4.97 (m, 2H), 4.73-4.32 (m, 2H), 4.02 (m, 2H), 3.83 (m, 12H), 3.51 (m, 1H), 3.35 (m, 4H), 2.13-1.79 (m, 6H), 1.47 (s, 9H); ¹³C NMR (100 MHz, DMSO-d₆) δ 166.0, 161.4, 158.4, 158.4, 154.4, 152.8, 149.4, 147.9, 132.8, 128.5, 122.8, 122.6, 122.3, 122.1, 122.1, 118.3, 117.8, 117.0, 114.2, 110.2, 104.6, 104.2, 103.7, 85.3, 79.1, 78.2, 66.6, 65.5, 60.5, 55.6, 45.9, 36.0, 35.9, 35.6, 29.0, 28.2, 22.7; IR (CHCl₃) 3315, 2976, 1698, 1633, 1587, 1515, 1465, 1435, 1405, 1314, 1269, 1206, 1163, 1105, 1061, 996, 754 cm⁻¹; MS (ES⁺) m/z (relative intensity) 872 ([M+H]^{+.}, 100).

### b) Preparation of 33

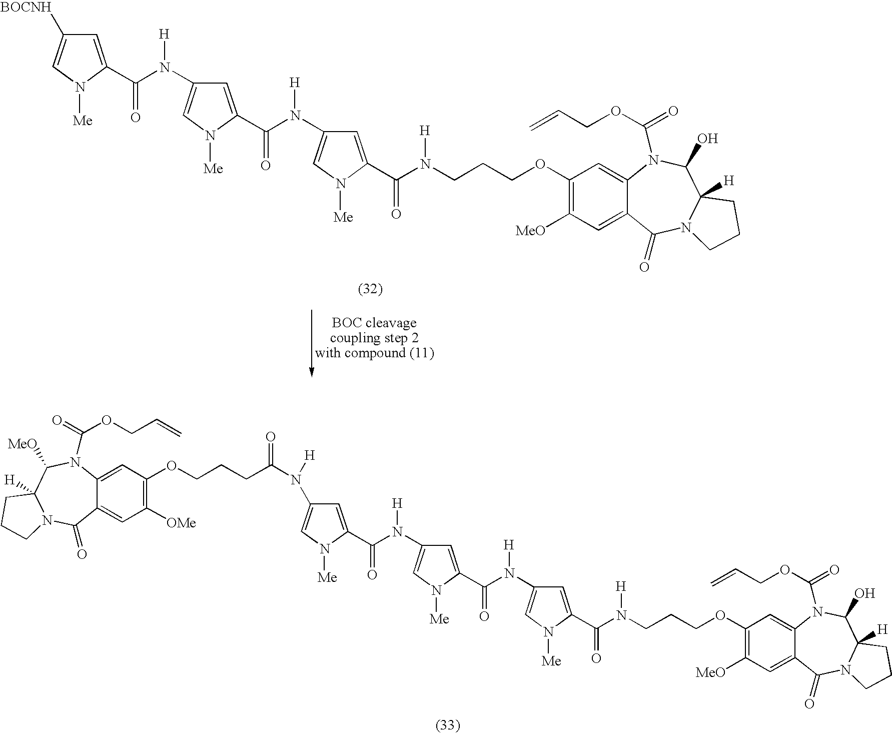

Finely ground 32 (180 mg, 0.206 mmol) was treated with a solution of 4N HCl in dioxane (8 mL) and stirred. After 1 h the solvent was evaporated in vacuo to provide a grey coloured salt which was dissolved in anhydrous THF (5 mL) in the presence of DIPEA (180 μL, 1.035 mmol). This mixture was treated dropwise with a pre-prepared (5 min stirring) solution of acid capping unit 11 (93 mg, 0.207 mmol), oxalyl chloride (27 μL, 0.228 mmol) and DMF (1 drop) in anhydrous THF (5 mL) at 0° C. (ice/acetone bath). After stirring for 1 h, the THF was removed in vacuo and residue dissolved in CHCl₃ (100 mL). The organic phase washed with 5% aqueous citric acid (50 mL), water (50 mL), sat^{d} aqueous NaHCO₃ (50 mL), brine (50 mL) dried (MgSO₄), filtered and the solvent removed in vacuo to provide a yellow oil. Purification by flash chromatography (gradient elution from 3:97 to 4:96 v/v MeOH/CHCl₃) yielded 33 (110 mg, 44%): ¹H NMR (400 MHz, DMSO-d₆) δ 9.89 (s, 3H), 8.09 (m, 1H), 7.25 (d, J=1.36 Hz, 1H), 7.19 (m, 2H), 7.11 (m, 2H), 7.05 (s, 1H), 6.92 (m, 3H), 6.79 (s, 1H), 6.53 (br s, 1H), 6.02-5.70 (m, 2H), 5.49 (m, 1H), 5.37 (d, J=8.8 Hz, 1H), 5.14-4.95 (m, 4H), 4.72-4.32 (m, 4H), 4.04 (m, 4H), 3.85 (m, 15H), 3.47 (m, 5H), 3.40-3.20 (m, 6H), 2.45 (t, J=7.04 Hz, 2H), 2.14-1.80 (m, 12H); ¹³C NMR (100 MHz, DMSO-d₆) δ 168.8, 166.1, 161.3, 158.4, 154.4, 148.3, 122.8, 122.7, 122.7, 122.1, 122.0, 118.1, 114.3, 104.6, 104.2, 92.9, 79.1, 55.6, 45.9, 36.0, 35.9, 22.8; MS (ES⁺) m/z (relative intensity) 1202 ([M+H]^{+.}, 40), 292 (100).

### c) Preparation of Dimer 18 (AT-235)

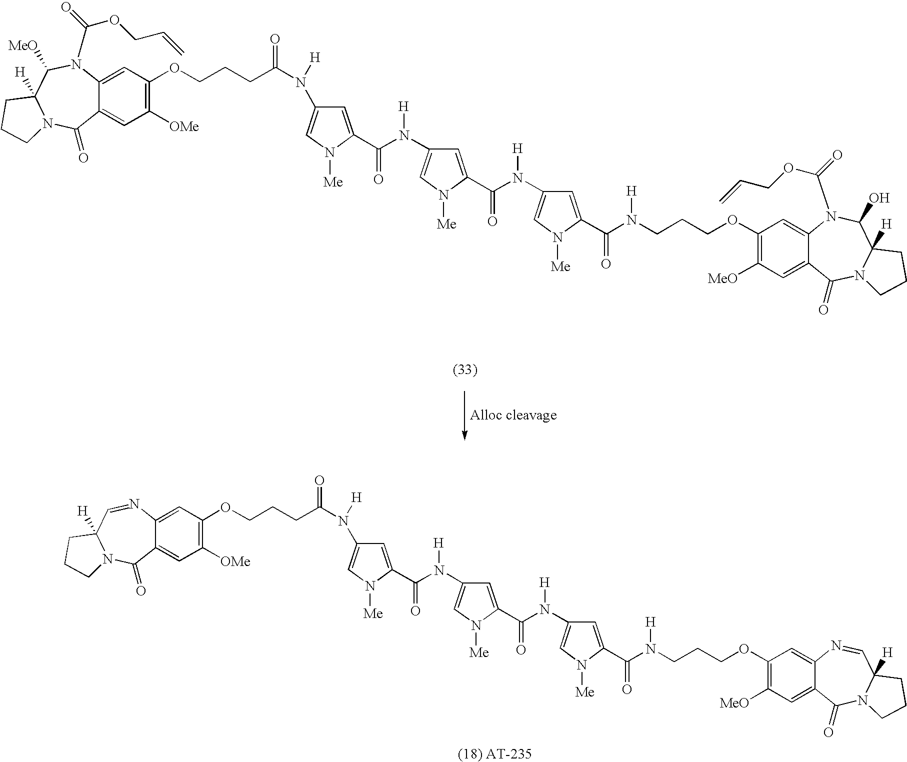

A solution of 33 (80 mg, 0.067 mmol) in anhydrous CHCl₃ (5 mL) was treated with Pd(PPh₃)₄ (1.5 mg, μmol) and pyrrolidine (11.7 μL, 0.140 mmol) and stirred. After 1 h stirring at room temperature reaction completion was observed by TLC. The solvent was removed in vacuo and the residue purified by flash chromatography (gradient elution from 5:95 to 7:93 v/v MeOH/CHCl₃) to yield 18 (AT-235) as an off-white solid (56 mg, 85%). A sample was dissolved in deuterated DMSO and NMR data of the diimine form was recorded after standing for at least 48 h: ¹H NMR (400 MHz, DMSO-d₆) (diimine only) δ 9.90 (s, 3H), 8.10 (m, 1H), 7.79 (d, 2H, J=4.36 Hz), 7.35 (s, 2H), 7.25 (s, 1H), 7.20 (2s, 2H), 7.05 (s, 1H), 6.90 (s, 2H), 6.84 (s, 2H), 4.21-4.01 (m, 4H), 3.85-3.81 (m, 15H), 3.61 (m, 2H), 3.46-3.35 (m, 6H), 2.45 (m, 2H), 2.38-2.17 (m, 4H), 2.13-1.89 (m, 8H); ¹³C NMR (100 MHz, DMSO-d₆) δ 168.8, 164.2, 158.4, 150.3, 150.2, 146.9, 140.5, 122.7, 122.1, 122.0, 119.7, 118.4, 118.4, 117.8, 114.9, 111.2, 110.1, 104.6, 104.2, 103.9, 67.8, 66.4, 55.8, 55.6, 53.7, 53.4, 48.5, 46.3, 36.0, 35.9, 35.6, 30.2, 29.0, 28.8, 24.7, 23.6, 22.4; MS (ES⁺) m/z (relative intensity) 984 ([M+H]^{+.}, 100); IR (CHCl₃) 3306, 2949, 1638, 1597, 1555, 1508, 1465, 1435, 1405, 1262, 1216, 1090, 1064 cm⁻¹.

### Example 12

### Synthesis of Dimer 49 (SJG-085)

### a) Alternative Method for the Preparation of Compound 26

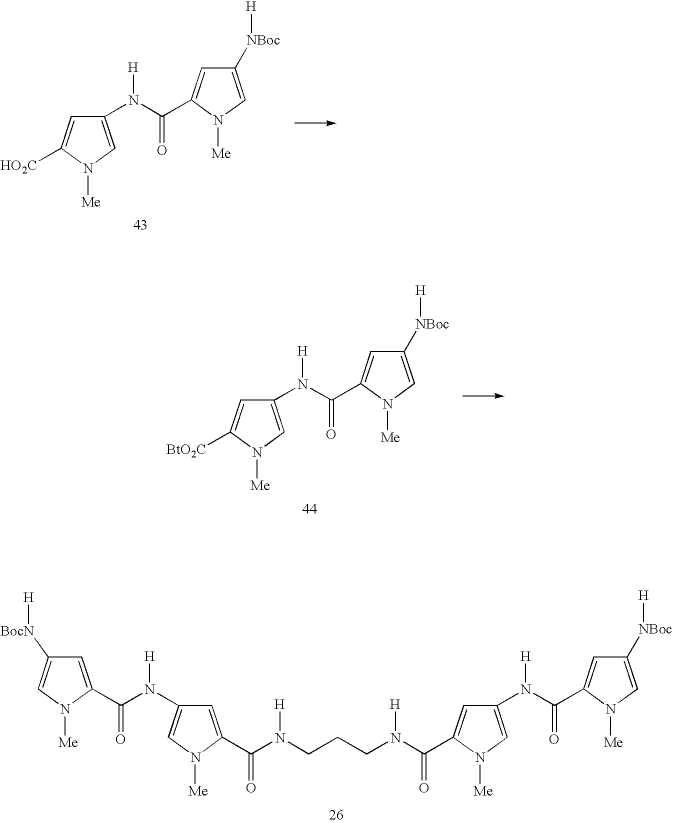

EDCI (671 mg, 3.50 mmol) was added to a stirred solution of the acid 43 (1.15 g, 3.18 mmol) in anhydrous DMF (12 mL) at room temperature. After 5 min stirring at room temperature, the reaction mixture was treated with HOBt (473 mg, 3.50 mmol) and allowed to stir under a nitrogen atmosphere for 3 h at which point TLC (EtOAc) revealed reaction completion and the formation of the desired benzotriazole (Bt) ester 44. The reaction mixture was treated with 1,3-diaminopropane (133 μL, 118 mg, 1.59 mmol) and allowed to continue stirring under nitrogen for 16 h. Following dilution with H₂O (600 mL), the mixture was extracted with CHCl₃ (120 mL). The organic layer washed with 1% aqueous citric acid (40 mL), saturated aqueous NaHCO₃ (40 mL), brine (40 mL), dried (MgSO₄), filtered and evaporated to provide the crude product. Purification by flash chromatography (EtOAc) provided the pure tetrapyrrole 26 as an orange oil (862 mg, 71%): LC/MS 3.38 min (ES+) m/z (relative intensity) 763 ([M+H]^{+.}, 100), 663 (22), 301 (49), 143 (29).

### b) Preparation of 45

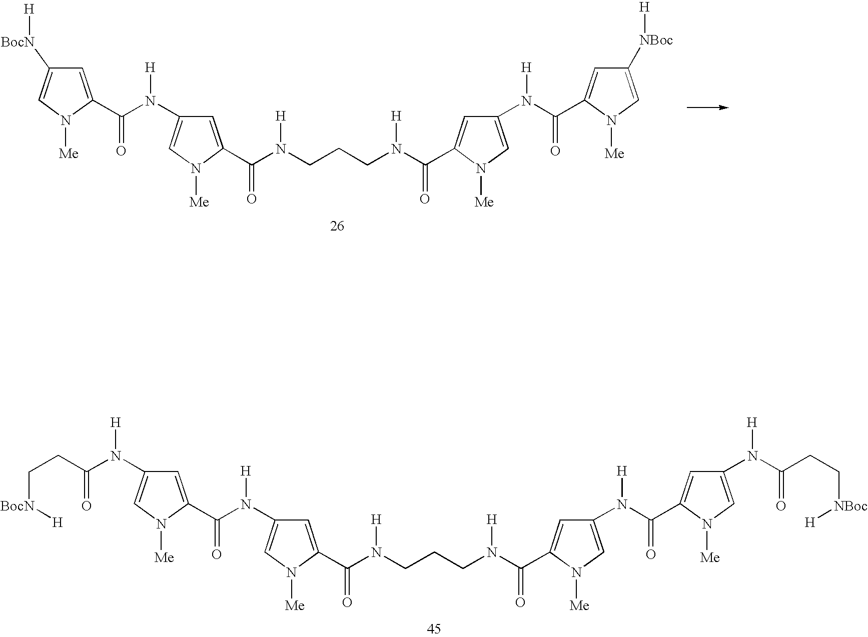

A solution of 4M HCl in dioxane (10 mL) was added to the bis-Boc protected tetrapyrrole 26 (862 mg, 1.13 mmol). The reaction failed to stir, however, the addition of THF (3 mL) assisted mobility. The slurry was allowed to stir at room temperature under a nitrogen atmosphere for 1 h, at which point TLC (EtOAc) revealed complete consumption of starting material. The solvent was removed by evaporation in vacuo to provide the pyrrole amine HCl salt 26a as a solid which was analysed by LC/MS (0.62 and 1.38 min gave identical spectra (ES⁺) m/z (relative intensity) 563 ([M+H]^{+.}, 100), 282 (19), 245 (50), 219 (30), 123 (52)) and carried through to the next step without further purification. In a separate vessel, EDCI (477 mg, 2.49 mmol) was added to a stirred solution of Boc-β-Ala-OH (428 mg, 2.26 mmol) in anhydrous DMF (6 mL) at room temperature. After 10 min stirring at room temperature, the reaction mixture was treated with HOBt (336 mg, 2.49 mmol) and allowed to stir under a nitrogen atmosphere for 3 h. The Boc-β-Ala-OBt ester solution was added to the pyrrole amine HCl salt 26a in the presence of DIPEA (0.434 mL, 0.322 g, 2.49 mmol) and the reaction mixture was allowed to stir under a nitrogen atmosphere. After 16 h stirring LC/MS (2.95 min (ES+) m/z (relative intensity) 905 ([M+H]^{+.}, 71), 805 (23), 372 (100)) revealed formation of the desired product. Following dilution with H₂O (600 mL), the mixture was extracted with CHCl₃ (105 mL). The organic layer washed with 1% aqueous citric acid (40 mL), saturated aqueous NaHCO₃ (40 mL), brine (40 mL), dried (MgSO₄), filtered and evaporated to provide the pure product 45 (812 mg, 79%): ¹H NMR (400 MHz, DMSO-d₆) δ 9.89 (s, 2H), 9.88 (s, 2H), 8.06 (t, 2H, J=5.7 Hz), 7.21 (d, 2H, J=1.6 Hz), 7.18 (d, 2H, J=1.5 Hz), 6.89-6.83 (m, 6H), 3.84 (s, 6H), 3.83 (s, 6H), 3.30-3.12 (m, 8H), 2.41 (t, 4H, J=7.3 Hz), 1.70 (p, 2H, J=6.6 Hz), 1.40 (s, 18H); ¹³C NMR (100 MHz, DMSO-d₆) δ 167.5, 161.3, 158.3, 155.5, 122.9, 122.7, 122.0, 121.9, 118.1, 117.8, 104.0, 103.8, 77.6, 36.7, 36.1, 36.0, 35.9, 29.7, 28.2; IR (ATR) 3298, 2930, 1650, 1579, 1518, 1463, 1433, 1386, 1365, 1252, 1206, 1164, 1095, 1062, 1005, 973, 775, 660 cm⁻¹.

### c) Preparation of 46

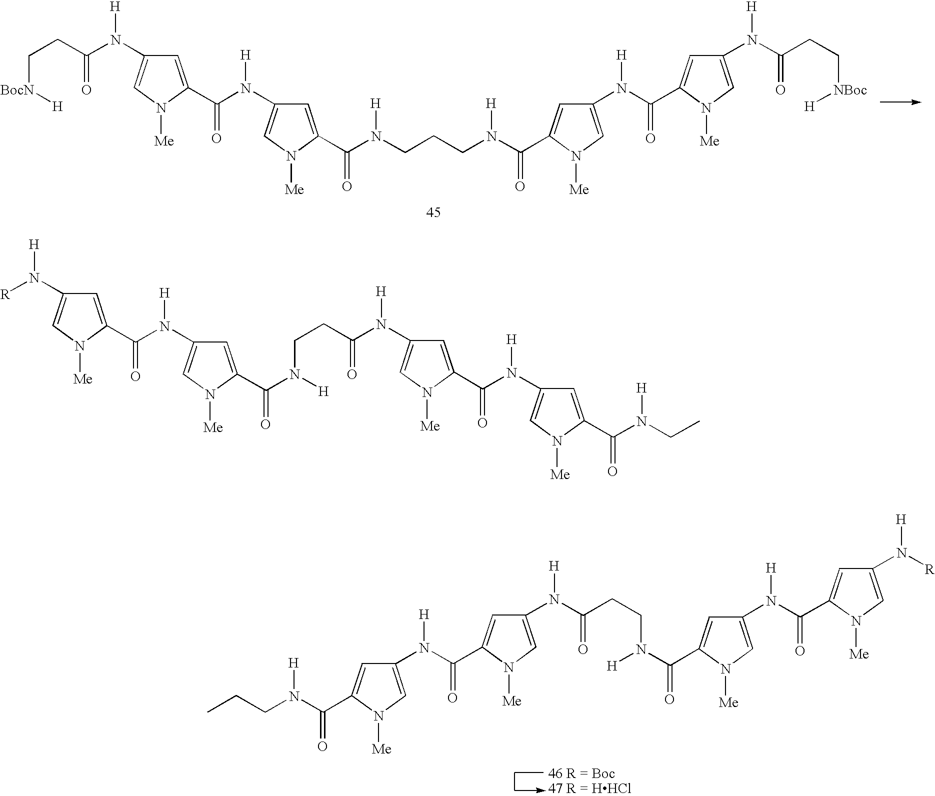

A solution of 4M HCl in dioxane (10 mL) was added to a stirred solution of the bis-Boc protected compound 45 (800 mg, 0.89 mmol) in THF (3 mL). The slurry was allowed to stir at room temperature under a nitrogen atmosphere for 1 h, at which point TLC (90:10 v/v CHCl₃/MeOH) revealed complete consumption of starting material. The solvent was removed by evaporation in vacuo to provide the pyrrole amine HCl salt 45a as a solid which was analysed by LC/MS (0.62 and 1.47 min gave identical spectra (ES⁺) m/z (relative intensity) 705 ([M+H]^{+.}, 15), 512 (10), 416 (30), 390 (43), 353 (80), 316 (48), 290 (82), 245 (38), 194 (100)) and carried through to the next step without further purification. In a separate vessel, EDCI (374 mg, 1.95 mmol) was added to a stirred solution of dipyrrole 43 (641 mg, 1.77 mmol) in anhydrous DMF (6 mL) at room temperature. After 5 min stirring at room temperature, the reaction mixture was treated with HOBt (263 mg, 1.95 mmol) and allowed to stir under a nitrogen atmosphere for 2.5 h at which point TLC (90:10 v/v CHCl₃/MeOH) revealed reaction completion and the formation of the desired benzotriazole (Bt) ester 44. The solution containing 44 was added to the pyrrole amine HCl salt 45a in the presence of DIPEA (0.337 mL, 0.252 g, 1.95 mmol) and the reaction mixture was allowed to stir under a nitrogen atmosphere. After 16 h stirring LC/MS (3.15 min (ES⁺) m/z (relative intensity) 1394 ([M+2H]^{+.}, 4), 716 (6), 597 (22), 143 (100)) and TLC (90:10 v/v CHCl₃/MeOH) revealed formation of the desired product. The reaction mixture was poured into H₂O (600 mL) which resulted in the formation of an orange precipitate which was collected by vacuum filtration and dried in the vacuum desicator to provide the crude product (922 mg). Purification by flash chromatography (gradient elution: 95:5 v/v CHCl₃/MeOH to 90:10 v/v CHCl₃/MeOH) provided the pure Boc-protected octapyrrole 46 as a solid (243 mg, 20%): ¹H NMR (400 MHz, DMSO-d₆) δ 9.93 (s, 2H), 9.90 (s, 2H), 9.84 (s, 2H), 9.11 (s, 2H), 8.08-8.06 (m, 4H), 7.25-7.16 (m, 6H), 6.90-6.79 (m, 10H), 3.85 (s, 6H), 3.82 (s×2, 12H), 3.81 (s, 6H), 3.52-3.39 (m, 4H), 3.24-3.18 (m, 4H), 2.55-2.51 (m, 4H, obscured by DMSO peak), 1.78-1.62 (m, 2H), 1.47 (s, 18H); ¹³C NMR (100 MHz, DMSO-d₆) δ 167.8, 161.3, 158.3, 152.8, 122.9, 122.7 (×2), 122.3, 122.1, 122.0, 121.9, 118.1, 117.8, 104.2, 104.0, 103.8, 103.7, 78.2, 36.1, 36.0, 35.9, 35.8, 35.5, 29.7, 28.2; IR (ATR) 3292, 2940, 1638, 1583, 1514, 1463, 1432, 1399, 1365, 1246, 1205, 1155, 1098, 1060, 998, 893, 747, 666 cm⁻¹.

### d) Preparation of 48

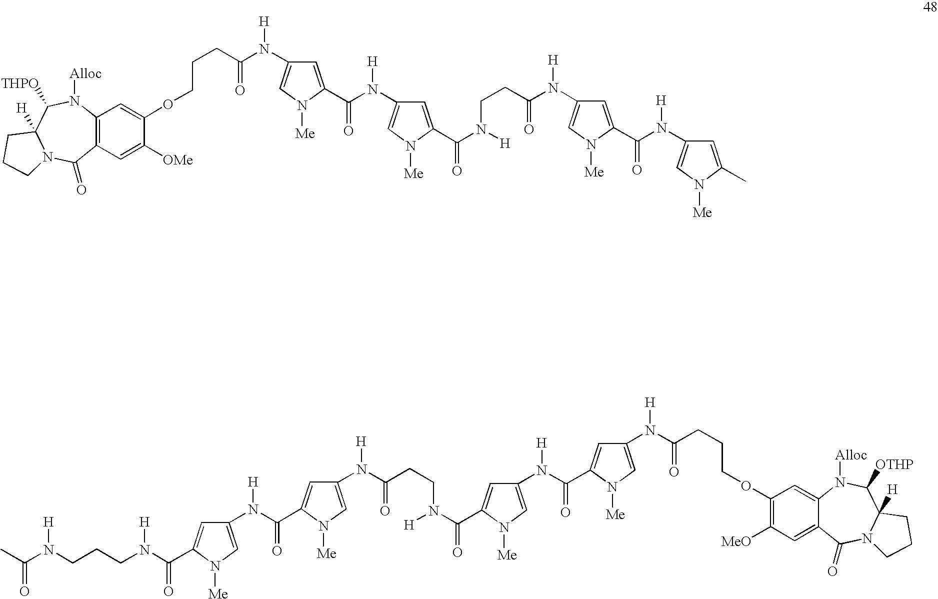

A solution of 4M HCl in dioxane (10 mL) was added to the bis-Boc protected octapyrrole 46 (191 mg, 0.14 mmol) The slurry was allowed to stir at room temperature under a nitrogen atmosphere for 1 h, at which point TLC (90:10 v/v CHCl₃/MeOH) revealed complete consumption of starting material. The solvent was removed by evaporation in vacuo to provide the pyrrole amine HCl salt 47 as a solid which was analysed by LC/MS (1.65 min (ES+) m/z (relative intensity) 1193 ([M+H]^{+.}, 3), 1071 (2), 975 (2), 617 (10), 597 (100), 534 (4), 412 (4), 123 (35)) and carried through to the next step without further purification. In a separate vessel, EDCI (58 mg, 0.30 mmol) was added to a stirred solution of PBD-acid 12 (142 mg, 0.27 mmol) in anhydrous DMF (5 mL) at room temperature. After 5 min stirring at room temperature, the reaction mixture was treated with HOBt (41 mg, 0.30 mmol) and allowed to stir under a nitrogen atmosphere for 16 h where TLC (90:10 v/v CHCl₃/MeOH) revealed reaction completion. The solution containing the Bt-ester of 12 was added to the pyrrole amine HCl salt 47 in the presence of DIPEA (52 μL, 38 mg, 0.30 mmol) and the reaction mixture was allowed to stir under a nitrogen atmosphere. After 16 h stirring LC/MS (3.17 min (ES⁺) m/z (relative intensity) 1117 (51), 1098 ([M+2H]^{2+.}, 100), 664 (15), 628 (10), 143 (80)) and TLC (90:10 v/v CHCl₃/MeOH) revealed formation of the desired product. Following dilution with H₂O (300 mL), the mixture was extracted with CHCl₃ (2×50 mL). The organic layer washed with 1% aqueous citric acid (30 mL), saturated aqueous NaHCO₃ (30 mL), brine (30 mL), dried (MgSO₄), filtered and evaporated to provide the crude product. Purification by flash chromatography (gradient elution: 98:2 v/v CHCl₃/MeOH to 92.5:7.5 v/v CHCl₃/MeOH) provided the pure bis-PBD-octapyrrole 48 as a solid (44.1 mg, 15%).

### e) Preparation of Dimer 49 (SJG-085)

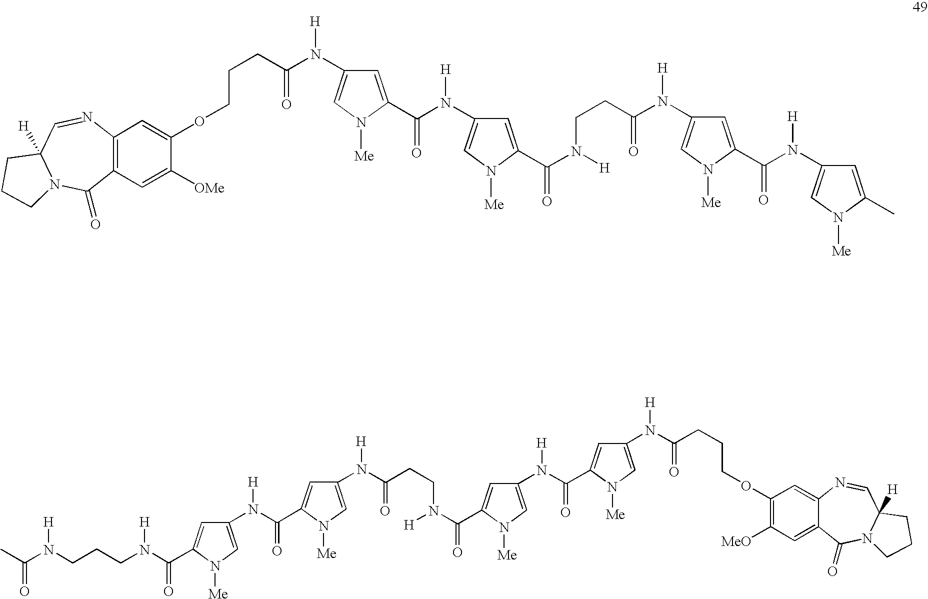

A catalytic amount of tetrakis(triphenylphosphine)palladium (1.13 mg, 0.98 μmol) was added to a stirred solution of the protected PBD dimer 48 (43.1 mg, 19.7 μmol), Ph₃P (0.5 mg, 1.97 μmol) and pyrrolidine (2.93 mg, 3.43 μL, 41.3 μmol) in CH₂Cl₂ (1.5 mL). The reaction mixture was allowed to stir under a N₂ atmosphere at room temperature and the progress of reaction monitored by TLC (90:10 v/v CHCl₃/MeOH), after 2.5 h the reaction was not complete. Additional tetrakis(triphenylphosphine)palladium (1.13 mg, 0.98 μmol) and pyrrolidine (2.93 mg, 3.43 μL, 41.3 μmol) were added and the reaction mixture stirred for a further 1 h, at which point the reaction was deemed complete by TLC. The solvent was evaporated under reduced pressure and the resulting residue subjected to flash chromatography (gradient elution: 95:5 v/v CHCl₃/MeOH to 90:10 v/v CHCl₃/MeOH) to give 49 (012-SJG-085-2) as a white solid (25.4 mg, 71%): [α]²⁴_{D}=+209° (c=0.0263, DMSO); ¹H NMR (500 MHz, DMSO-d₆) δ 9.94 (s, 2H), 9.91 (s, 4H), 9.89 (s, 2H), 8.10 (t, 2H, J=5.6 Hz), 8.07 (t, 2H, J=5.7 Hz), 7.79 (d, 2H, J=4.4 Hz), 7.35 (s, 2H), 7.26-7.18 (m, 6H), 6.88-6.84 (m, 12H), 4.16-4.11 (m, 2H), 4.07-4.02 (m, 2H), 3.84 (s×2, 12H), 3.83 (s, 6H), 3.82 (s×2, 12H), 3.71-3.66 (m, 2H), 3.64-3.58 (m, 2H), 3.48-3.43 (m, 4H), 3.40-3.33 (m, 2H, obscured by H₂O peak), 3.26-3.20 (m, 4H), 2.56-2.50 (m, 4H, obscured by DMSO peak), 2.45 (t, 4H, J=7.3 Hz), 2.33-2.19 (m, 4H), 2.05 (p, 4H, J=6.9 Hz), 1.98-1.93 (m, 4H), 1.69 (p, 2H, J=6.7 Hz); ¹³C NMR (125 MHz, DMSO-d₆) δ 168.5, 164.1, 163.0, 158.0, 149.9, 141.1, 122.4, 121.7, 119.5, 117.8, 117.6, 110.8, 109.7, 104.1, 103.8, 67.5, 55.3, 53.1, 46.1, 35.9, 35.8, 35.7, 35.6, 35.4, 31.6, 28.5, 24.4, 23.4; LC/MS 2.42 min (ES⁺) m/z (relative intensity) 912 ([M+2H]^{2+.}, 100), 621 (10), 425 (5), 377 (4), 162 (40), 143 (90).

### Example 13

### Synthesis of Dimer 55 (AT-338)

### a) Preparation of 52

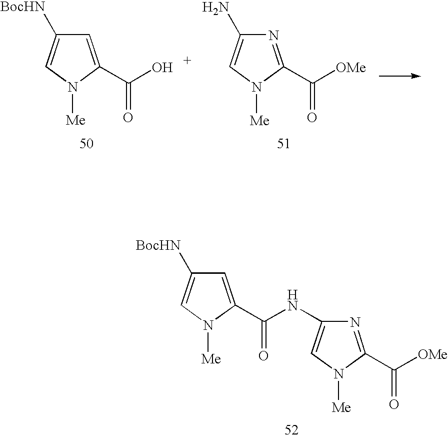

A suspension of imidazole amine 51 (1.80 g, 11.6 mmol), Boc pyrrole acid 50 (2.78 g, 11.6 mmol), EDCI (2.90 g, 15.1 mmol), and DMAP (280 mg, 2.29 mmol) in dry DMF (7 mL) was allowed to stir overnight at room temperature and then for a further 6 h at 60° C. The viscous solution was added dropwise to a stirred mixture of ice/water (300 mL) and the resulting suspension was allowed to stir for 30 min. The precipitate was collected by vacuum filtration, washed with water, and dried. The methyl ester thus obtained was dissolved in MeOH (40 mL) and treated with an aqueous solution of NaOH (0.77 g, 19.3 mmol, 15 mL). The mixture was stirred and heated at 60° C. for 4 h, at which point TLC revealed completion of the reaction. The reaction mixture was diluted with water (20 mL) and the methanol removed by evaporation in vacuo. The aqueous solution was then acidified to pH 4 with cold 1N HCl. The resulting precipitate was collected by vacuum filtration, washed with water and dried in a desiccator to yield 3.06 g (72%) of 52: ¹H NMR (400 MHz, DMSO-d₆) δ 10.56 (s, 1H), 9.08 (s, 1H), 7.59 (s, 1H), 7.00 (s, 1H), 6.93 (s, 1H), 3.94 (s, 3H), 3.83 (s, 3H), 1.46 (s, 9H); ¹³C NMR (100 MHz, DMSO-d₆) δ 160.1, 158.6, 152.8, 137.2, 132.1, 122.3, 121.9, 118.0, 114.7, 104.9, 78.2, 36.1, 35.4, 28.1; MS (ES⁺) m/z (relative intensity) 364 ([M+H]^{+.}, 80), 320.1 (100).

### b) Preparation of 53

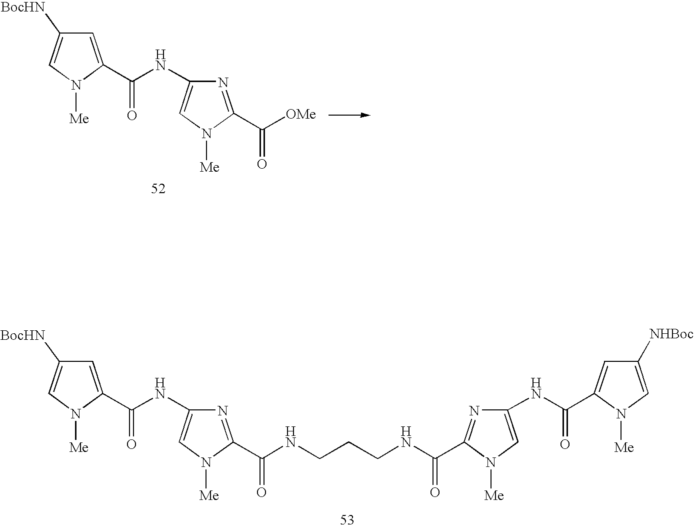

A solution of 52 (580 mg, 1.59 mmol), EDCI (367 mg, 1.91 mmol), DMAP (39 mg, 0.32 mmol) and 1,3-diaminopropane (73 μL, 0.87 mmol) in anhydrous DMF (6 mL) was allowed to stir overnight. Once reaction was complete as observed by TLC the mixture was poured into water (50 mL) with vigorous stirring. The precipitate was collected by vacuum filtration and re-dissolved in chloroform (50 mL). The crude product was absorbed onto silica gel and subjected to column chromatography (silica gel, 98:2 v/v CHCl₃:MeOH) to yield 400 mg (33%) of pure 53. ¹H NMR (400 MHz, DMSO-d₆) δ 10.18 (s, 2H), 9.07 (s, 2H), 8.11 (t, 2H, J=6.05 Hz), 7.48 (s, 2H), 7.00 (s, 2H), 6.89 (s, 2H), 3.95 (s, 6H), 3.83 (s, 6H), 3.30 (q, 4H, J=6.34 Hz), 1.60 (p, 2H, J=6.53 Hz), 1.47 (s, 18H); ¹³C NMR (100 MHz, DMSO-d₆) δ 158.9, 158.7, 152.8, 136.1, 133.9, 122.4, 121.9, 117.9, 114.3, 104.8, 78.2, 36.1, 35.9, 34.8, 30.7, 29.5, 28.2; MS (ES⁺) m/z (relative intensity) 765.4 ([M+H]^{+.}, 100).

### c) Preparation of 54

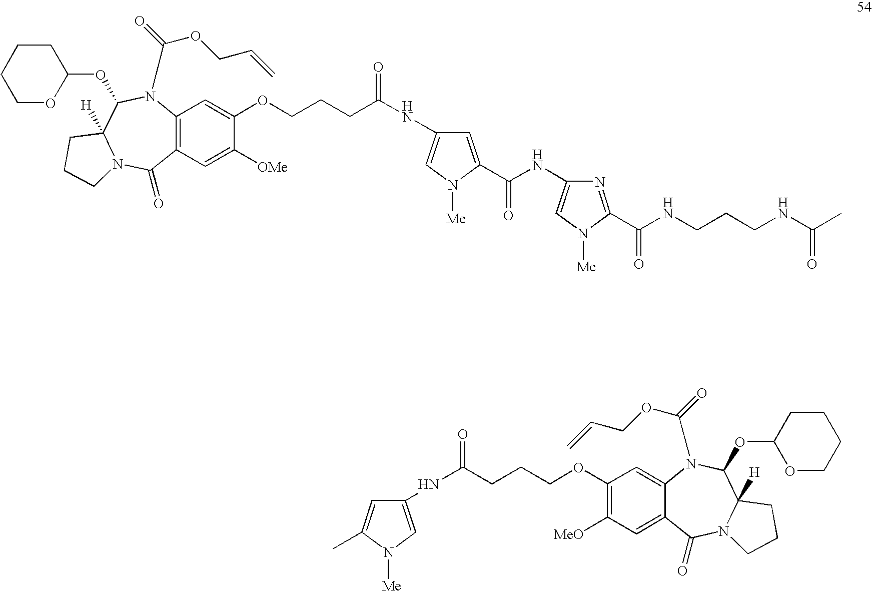

A suspension of 53 (350 mg, 0.457 mmol) in a solution of 4N HCl-dioxane (15 mL), was stirred for 1 h and subjected to ultrasound for 3 min. The solvent was removed by evaporation in vacuo to provide the pyrrole amine HCl salt as a solid, which was carried through to the next step without further purification. In a separate vessel, EDCI (180 mg, 0.94 mmol) and DMAP (160 mg, 1.31 mmol) were added to a stirred solution of the PBD acid (393 mg, 0.78 mmol) in anhydrous DMF (5 mL) at room temperature. The reaction mixture was added to the vessel containing the HCl salt and allowed to stir at room temperature over night. The reaction mixture was diluted with CHCl₃ (100 mL) and the organic phase was washed sequentially with water (50 mL), saturated aqueous NaHCO₃ (50 mL), brine (50 mL) and dried over MgSO₄. Excess solvent was removed under vacuum to afford a yellow oil, which was purified by flash chromatography (gradient from 0:100 to 4:96 v/v MeOH/CHCl₃) to yield pure 54 (170 mg, 33%) which was used immediately in the next reaction.

### d) Preparation of Dimer 55 (AT-338)

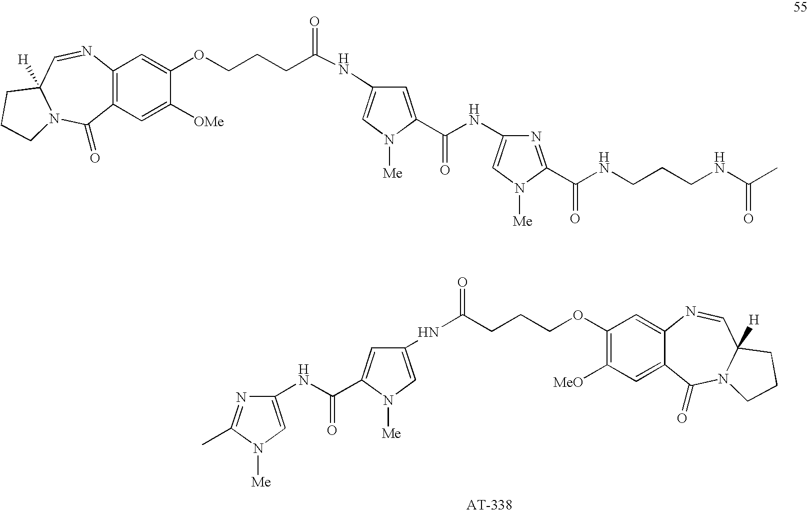

A catalytic amount of tetrakis(triphenylphosphine)palladium (2.3 mg, 2.0 μmol) was added to a stirred solution of the protected PBD dimer 54 (153 mg, 0.1 mmol), and pyrrolidine (17.2 μL, 0.21 mmol) in CHCl₃ (5 mL). The reaction mixture was allowed to stir under a N₂ atmosphere at room temperature and the progress of reaction monitored by TLC. After 2 h stirring at room temperature, the reaction was deemed complete by TLC. The solvent was evaporated under reduced pressure and the resulting residue subjected to flash chromatography (gradient elution: 98:2 v/v CHCl₃/MeOH to 92:8 v/v CHCl₃/MeOH) to yield 55 as an off white solid (78 mg, 67%): ¹H NMR (400 MHz, DMSO-d₆) 10.25 (s, 2H), 9.89 (s, 2H), 8.10 (t, 2H, J=6.1 Hz), 7.79 (d, 2H, J=4.4 Hz), 7.50 (s, 2H), 7.35 (s, 2H), 7.28 (d, 2H, J=1.7 Hz), 6.94 (d, 2H, J=1.8 Hz), 6.84 (s, 2H), 4.21-4.00 (m, 4H), 3.95 (s, 6H), 3.84 (s, 12H), 3.75-3.55 (m, 4H), 3.47-3.35 (m, 2H), 3.30 (m, 4H), 2.45 (t, 4H, J=7.3 Hz), 2.32-2.20 (m, 4H), 2.05 (p, 4H, J=6.5 Hz), 1.95 (m, 4H), 1.73 (m, 2H); MS (ES⁺) m/z (relative intensity) 1193.6 ([M+H]^{+.}, 100); HRMS (TOF MS ES⁺) calcd for C₅₉H₆₈N₁₆O₁₂ (M+H): 1193.5276. Found: 1193.5259.

### Example 14

### Synthesis of Dimer 62 (GDK-109)

### a) Preparation of 57

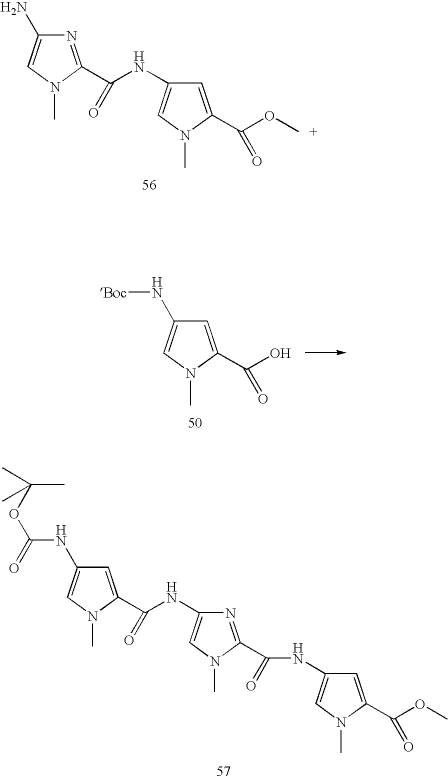

The amino compound 56 (0.2 g, 0.72 mmol; J. Am. Chem. Soc., 125, 12, 2003, 3471-3485), was added to a solution of 50 (0.19 g 0.79 mmol; J. Org. Chem., 66, 20, 2001, 6654-6661), EDCI (0.15 g, 0.79 mmol), and DMAP (44 mg, 0.36 mmol) in DMF (5 mL) and then stirred over night. The excess solvent was evaporated under reduced pressure and the residue was diluted with CHCl₃ (100 mL) and was washed sequentially with 5% aqueous citric acid (30 mL), water (30 mL), saturated aqueous NaHCO₃ (30 mL), brine (30 mL) and dried over MgSO₄. The solvents were removed under vacuum to leave a yellow oil, which was further purified by flash chromatography (50% EtOAc/Hexane) to yield compound 57 (250 mg, 69%): ¹H NMR (CDCl₃, 400 MHz): δ 8.91 (s, 1H), 8.08 (s, 1H), 7.45 (s, 1H), 7.44 (s, 1H), 6.99 (s, 1H), 6.86 (d, 1H, J=1.96 Hz), 6.59 (s, 1H), 6.50 (s, 1H), 4.05 (s, 3H), 3.91 (s, 6H), 3.83 (s, 3H); ¹³C NMR (CDCl₃, 100 MHz) δ 161.5, 158.3, 155.3, 153.3, 136.0, 133.7, 122.2, 122.0, 121.2, 120.7, 120.0, 119.1, 113.9, 108.2, 103.7, 80.3, 51.2, 36.8, 36.7, 35.7, 28.3; IR (neat): 3330, 2953, 1701, 1531, 1446, 1390, 1367, 1302, 1249, 1198, 1157, 1117, 1062, 997, 901, 775 cm⁻¹; MS (ES⁺) m/z (relative intensity) 500 ([M+H]^{+.}, 100), 501 ([M+2H]^{+.}, 20).

### b) Preparation of 58

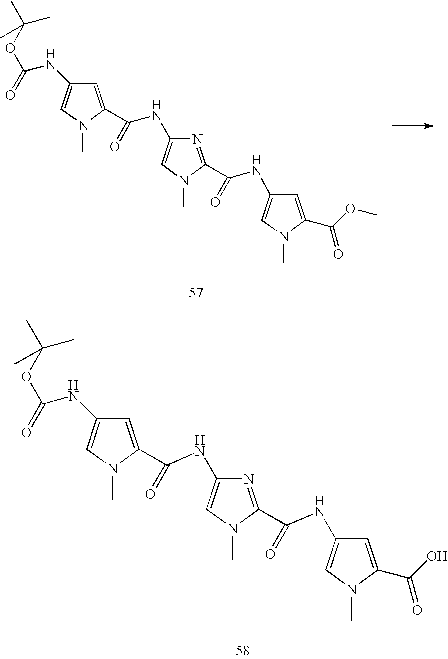

To a solution of ester compound 57 (0.1 g, 0.2 mmol) in dioxane (15 mL), was added 1N-NaOH (0.8 mL, 0.8 mmol) and then heated at reflux for 2 h. The reaction was monitored by TLC, and the excess solvent was removed under reduced pressure. The residue was redissolved into water and was acidified with 1N-HCl to afford a precipitate, which was collected by vacuum filtration and dried to give a brown solid 58 (91 mg, 93%): ¹H NMR (DMSO-d₆, 400 MHz) δ 12.15 (br 5, 1H), 10.10 (5, 1H), 10.08 (s, 1H), 9.08 (s, 1H), 7.51 (5, 1H), 7.47 (d, H, J=1.87 Hz), 6.98 (s, 1H), 6.94 (d, 1H), 6.85 (5, 1H), 3.97 (s, 3H), 3.83 (s, 6H), 1.45 (5, 9H); ¹³C NMR (DMSO-d₆, 100 MHz) δ 161.8, 158.6, 155.7, 153.1, 136.0, 133.9, 122.4, 121.9, 121.7, 120.3, 119.8, 114.6, 108.6, 103.7, 77.5, 36.1, 36.0, 34.8, 28.1; IR (neat): 3299, 2960, 2359, 2338, 1668, 1590, 1553, 1407, 1365, 1244, 1164, 1121, 1064, 1033, 991. 924, 776, 668, 629 cm¹; MS (ES⁺) m/z (relative intensity) 486 ([M+H]^{+.}, 100), 487 ([M+2H]^{+.}, 30).

### c) Preparation of 59

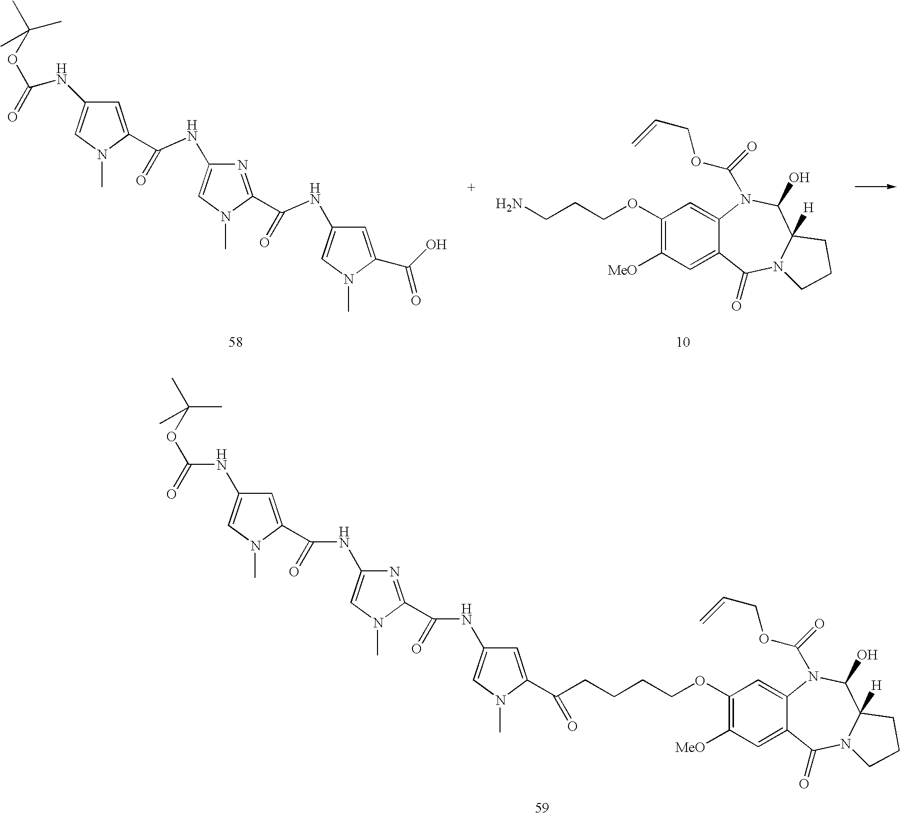

A solution of compound 58 (310 mg, 0.63 mmol), EDCI (134 mg, 0.70 mmol) and HOBt (100 mg, 0.70 mmol) in anhydrous DMF (5 mL) was stirred for 2 h. A solution of the amine capping unit (10) (284 mg, 0.70 mmol) in anhydrous DMF (5 mL) was then added and the resulting mixture was allowed to stir overnight at RT. The excess solvent was evaporated under reduced pressure and the residue was diluted with CHCl₃ (200 mL) and was sequentially washed with 5% aqueous citric acid (50 mL), water (50 mL), saturated aqueous NaHCO₃ (50 mL), brine (50 mL) and dried over MgSO₄. The solvents were removed under vacuum to leave a yellow oil, which was further purified by flash chromatography (EtOAc) to yield compound 59 (340 mg, 60%): [α]²⁴_{D}=+42° (c=0.14, CHCl₃); ¹H NMR (CDCl₃, 400 MHz): δ 8.96 (s, 1H), 8.64 (s, 1H), 7.46 (s, 1H), 7.30 (m, 1H), 7.26 (s, 1H), 7.16-7.02 (m, 2H), 6.89-6.65 (m, 3H), 6.44 (d, J=1.65 Hz, 1H), 5.84-5.63 (m, 2H), 5.16-4.97 (m, 2H), 4.69-4.65 (m, 1H), 4.50-4.41 (m, 1H), 4.18-4.02 (m, 5H), 3.84-3.96 (m, 9H), 3.78-3.43 (m, 5H), 2.15-1.90 (m, 6H), 1.48 (s, 9H); ¹³C NMR (100 MHz, CDCl₃) δ 166.9, 162.1, 158.3, 155.6, 152.8, 149.4, 147.9, 136.2, 132.8, 131.7, 128.5, 123.7, 122.5, 121.9, 120.8, 119.0, 118.2, 116.1, 114.1, 114.0, 110.7, 104.2, 103.1, 87.0, 86.1, 83.6, 81.0, 80.1, 69.0, 66.9, 61.8, 60.1, 56.0, 51.2, 46.5, 37.9, 36.8, 36.4, 35.6, 28.6, 28.4, 22.9; IR (neat): 3314, 2948, 1702, 1633, 1599, 1528, 1463, 1434, 1405, 1366, 1274, 1242, 1202, 1162, 1107, 1056, 1015, 995, 911, 775, 729, 646 cm⁻¹; MS (ES⁺) m/z (relative intensity) 873 ([M+H]^{+.}, 100), 874 ([M+2H]^{+.}, 50).

### d) Preparation of 61

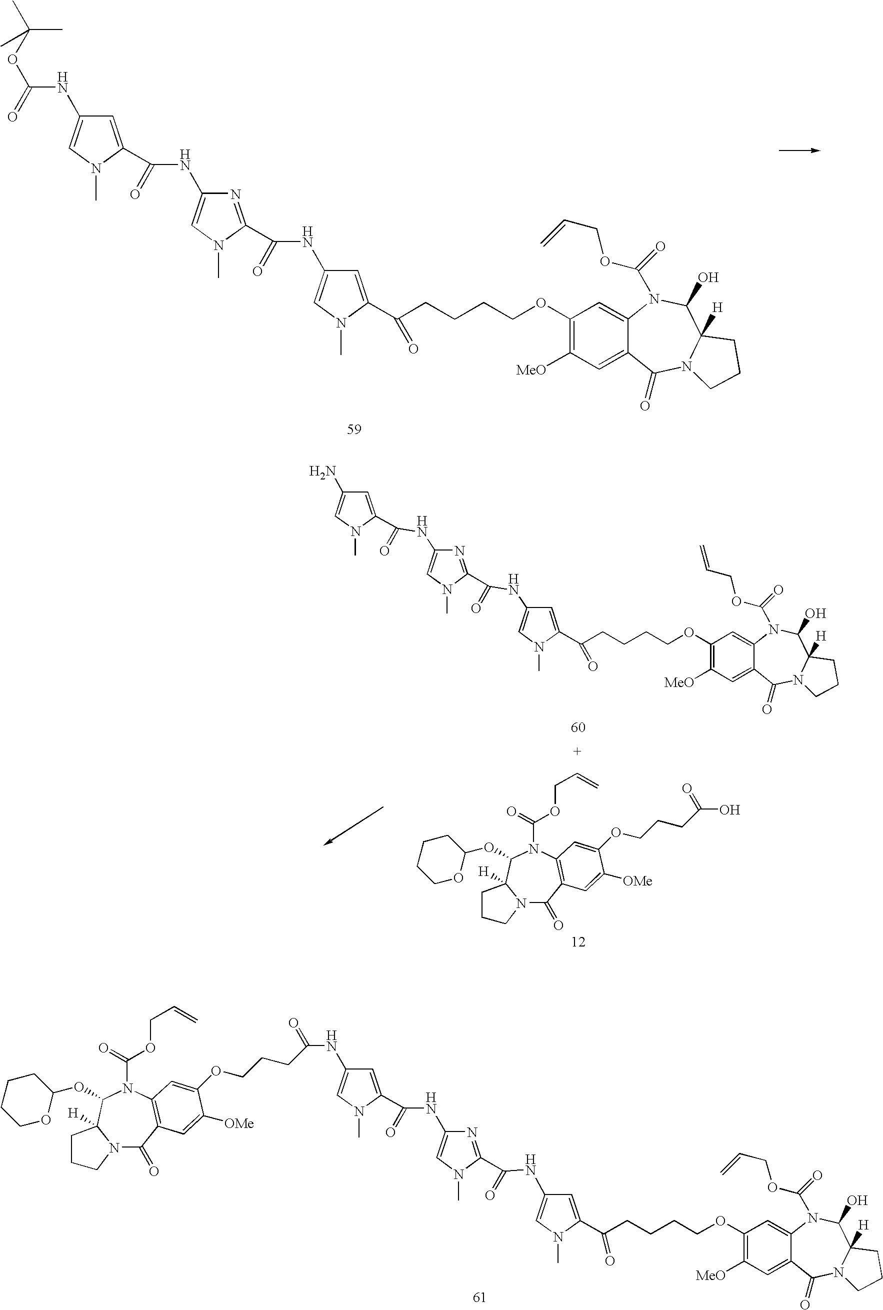

Compound 59 (340 mg, 0.38 mmol) was suspended in a solution of 4N HCl in dioxane (5 mL) and stirred for 1 h. Following evaporation of solvent in vacuo the grey salt 60 was treated with anhydrous DMF (5 mL) in the presence of DIPEA (338 μL, 1.94 mmol). In a separate flask, acid capping unit 12 (221 mg, 0.42 mmol) was dissolved in anhydrous DMF (5 mL), treated with EDCI (82 mg, 0.42 mmol), and stirred for 10 min at 0° C. The reaction mixture was treated with HOBt (65.6 mg, 0.42 mmol) and stirred for 3 h and the solution containing compound 60 was added dropwise and then stirred over night. The DMF was removed in vacuo and residue dissolved in CHCl₃ (100 mL) and washed with 5% aqueous citric acid (50 mL), water (50 mL), saturated aqueous NaHCO₃ (50 mL), brine (50 mL) and dried over MgSO₄. The solvents were removed under vacuum to leave a yellow oil which was further purified by flash chromatography (gradient from 2% MeOH/CHCl₃) to yield compound 61 (200 mg, 39%): [α]²³_{D}=+35° (c=0.14, CHCl₃); ¹H NMR (CDCl₃, 400 MHz) δ 9.22-9.07 (m, 1H), 8.29-8.05 (m, 2H), 7.50-7.16 (m, 10H), 7.00-6.84 (m, 2H), 6.81-6.47 (m, 4H), 6.05-5.46 (m, 6H), 5.37-4.96 (m, 6H), 4.75-4.56 (m, 4H), 4.52-4.30 (m, 2H), 4.29-4.02 (m, 10H), 4.00-3.35 (m, 36H), 2.73-2.50 (m, 3H), 2.34-1.87 (m, 19H), 1.85-1.66 (m, 6H), 1.65-1.45 (m, 7H); IR (neat): 3314, 2947, 2876, 1708, 1641, 1602, 1513, 1453, 1432, 1405, 1311, 1270, 1201, 1112, 1018, 774, 729, 646 cm⁻¹.

### e) Preparation of Dimer 62 (GDK-109)

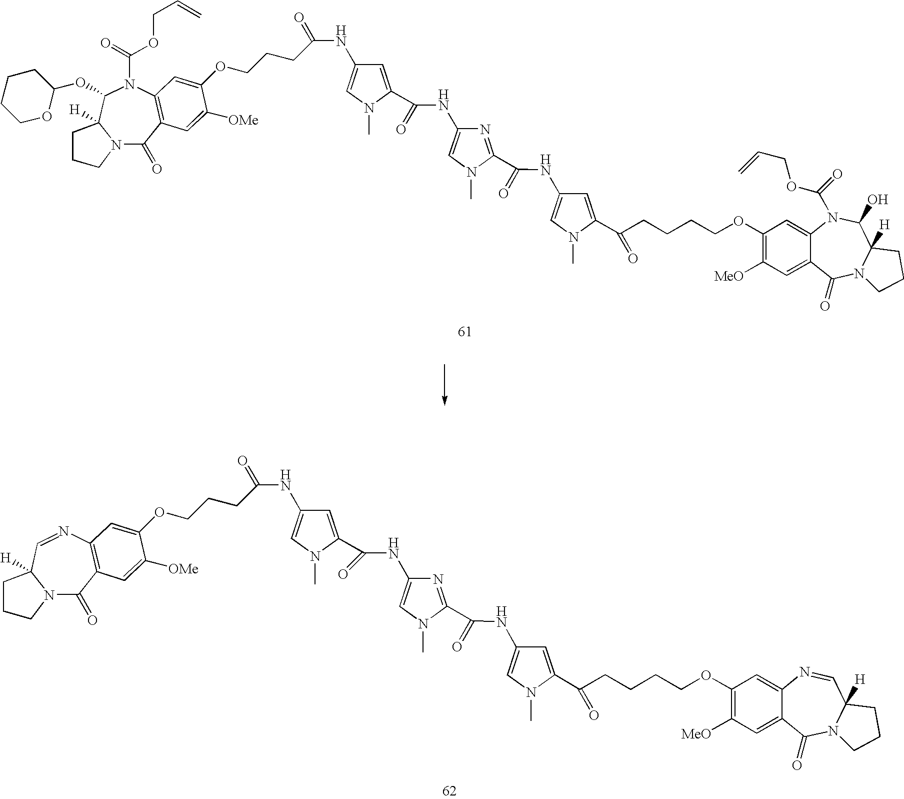

Compound 61 (200 mg, 0.15 mmol), Pd(PPh₃)₄ (3.5 mg, 3 μmol) and pyrrolidine (26 μL, 0.31 mmol) were stirred in anhydrous CHCl₃ (15 mL). Reaction completion was reached in 1 h as observed by TLC. The solvent was removed under vacuum and the residue purified by flash chromatography (gradient from 3:97 to 4:96 MeOH/CHCl₃) to afford final compound 62 as an off white solid (130 mg, 88%): [α]²⁴_{D}=+353° (c=0.13, CHCl₃); ¹H NMR (CD Cl₃, 400 MHz) δ 8.93 (s, 1H), 8.69 (s, 1H), 8.60 (s, 1H), 7.68 (d, 1H, J=4.45 Hz), 7.63-7.62 (m, 2H), 7.48 (s, 1H), 7.43-7.42 (m, 2H), 7.39 (d, 1H, J=1.46 Hz), 7.30 (d, 1H, J=1.59 Hz), 6.91-6.85 (m, 2H), 6.79 (s, 1H), 6.68 (d, 1H, J=1.41 Hz), 6.22 (d, 1H, J=1.55 Hz), 4.35-4.07 (m, 4H), 4.06-3.83 (m, 15H), 3.82-3.44 (m, 8H), 2.63-2.46 (m, 2H), 2.34-2.13 (m, 8H), 2.06-1.84 (m, 4H); ¹³C NMR (CDCl₃,100 MHz) δ 169.6, 164.8, 164.6, 162.8, 162.5, 161.6, 158.5, 155.6, 150.6, 150.3, 147.7, 147.6, 141.0, 140.7, 136.3, 133.7, 123.8, 122.0, 121.7, 120.7, 120.5, 120.4, 118.0, 113.6, 111.6, 111.2, 110.9, 110.3, 104.0, 102.8, 68.9, 68.0, 56.1, 56.0, 53.9, 53.7, 50.8, 46.8, 46.6, 38.2, 36.8, 36.3, 35.5, 32.9, 29.5, 29.4, 28.1, 24.9, 24.1; IR (neat): 3280, 2948, 1596, 1529, 1503, 1462, 1451, 1430, 1403, 1383, 1259, 1214, 1093, 1017, 908, 874, 771, 724, 664, 645 cm⁻¹; MS (ES⁺) m/z (relative intensity) 985([M+H]^{+.}, 100), 986 ([M+2H]^{+.}, 60).

### Example 15

### Synthesis of Bisulphite Salt 63 (AT-361)

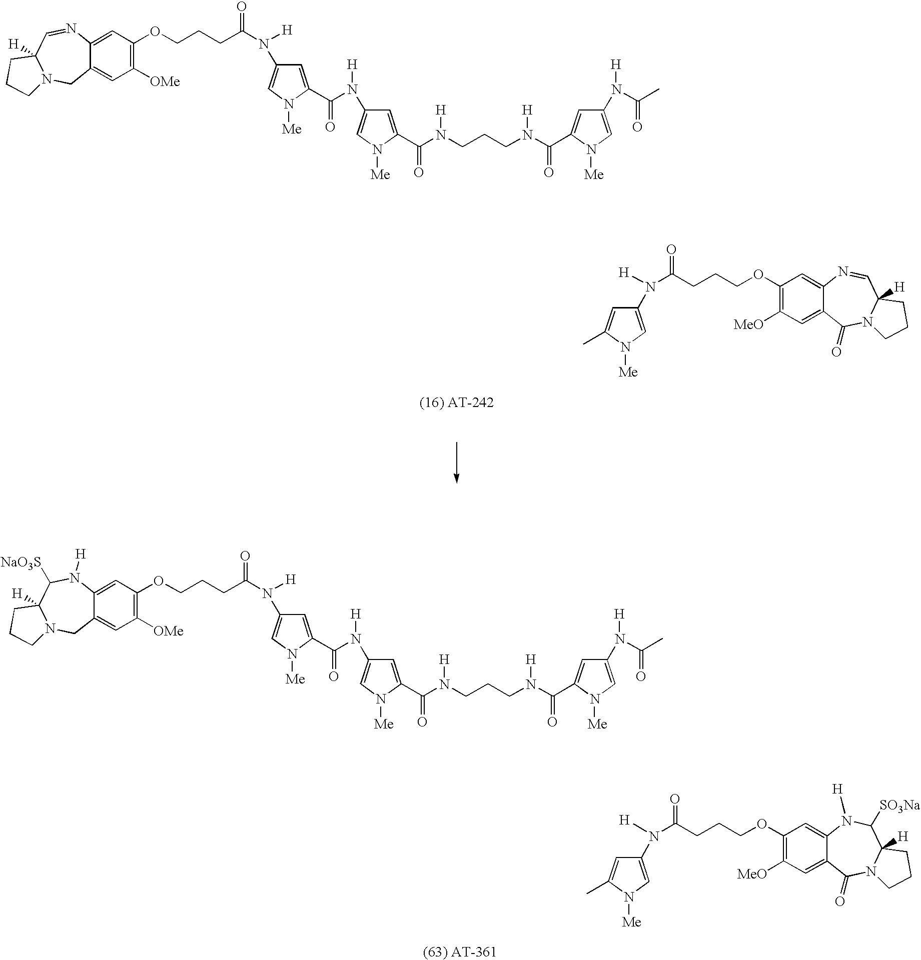

A solution of sodium bisulphite (12.6 mg, 0.12 mmol) in water (2 mL) was added to a stirred solution of 16 (AT-242) (72.0 mg, 60.4 . . . mol) in dichloromethane (2.0 mL). The reaction mixture was allowed to stir vigorously for 5 h, after which time the organic and aqueous layers were separated. TLC analysis (eluent-95:5 v/v CHCl₃/MeOH) of the aqueous phase revealed absence of AT-242 and presence of baseline material with strong UV absorption. The aqueous layer was lyophilised to provide the bisulphite adduct 63 (AT-361) as a lightweight white solid (59.2 mg, 70%): ¹H NMR (CDCl₃, 400 MHz) δ 9.89 (m, 4H), 8.03 (t, 2H, J=5.61 Hz), 7.19 (dd, 4H, J=10.4, 1.67 Hz), 7.02 (s, 2H), 6.90 (m, 4H), 6.40 (s, 2H), 5.04 (s, 2H), 3.98 (m, 4H), 3.84-3.77 (m, 14H), 3.70 (s, 6H), 3.51-3.43 (m, 4H), 3.26-3.21 (m, 4H), 2.56 (m, 2H), 2.44 (t, 4H, J=7.27 Hz), 2.11-1.60 (m, 14H); ¹³C NMR (CDCl₃, 100 MHz) δ 168.8, 167.1, 161.3, 158.4, 150.8, 142.8, 140.1, 122.9, 122.7, 122.1, 122.0, 118.1, 117.8, 116.9, 112.6, 106.4, 104.1, 78.8, 67.6, 56.5, 55.8, 46.1, 36.0, 35.9, 31.9, 29.4, 22.6.

### Example 16

### Determination of DNA Cross-Linking Ability and in Vitro Cytotoxicity

### (a) DNA Cross-Linking

The extent of DNA cross-linking induced by each PBD dimer was determined using the electrophoretic assay method of Hartley, et al. (Hartley, J. A., Berardini, M. D., and Souhami, R. L. (1991) Anal. Biochem. 193, 131-134) based on the principle that, following complete denaturation of linear pBR322 DNA (˜4,300 bp) to the single-stranded (SS) form, an interstrand cross-link results in renaturation to double-stranded (DS) in a neutral gel.

Closed-circular DNA was linearized with HindIII, then dephosphorylated and finally 5′-singly end-labelled using [γ³²P]-ATP and polynucleotide kinase. Reactions containing 30-40 ng of DNA and the test compound were carried out in aqueous TEOA (25 mM triethanolamine, 1 mM EDTA, pH 7.2) buffer at 37° C. in a final volume of 50 μl for 2 h. Reactions were terminated by addition of an equal volume of stop solution (0.6 M NaOAc, 20 mM EDTA, 100 μg/ml tRNA) followed by precipitation with ethanol. Following centrifugation, the supernatant was discarded and the pellet dried by lyophilization. Samples were re-suspended in 10 μl of strand separation buffer (30% DMSO, 1 mM EDTA, 0.04% bromophenol blue and 0.04% xylene cylanol) and denatured by heating to 90° C. for 2.5 min, followed by immersion in an ice/water bath. Control non-denatured samples were re-suspended in 10 μl of non-denaturing buffer solution (0.6% sucrose, 0.04% bromophenol blue in aqueous TAE buffer [40 mM Tris, 20 mM acetic acid, 2 mM EDTA, pH 8.1]) and loaded directly onto the gel for comparison.

Electrophoresis was carried out for 14-16 h at 40 V using a 0.8% submerged agarose gel (20×25×0.5 cm) in TAE buffer. Gels were dried under vacuum for 2 h at 80° C. onto one layer each of Whatman 3MM and DE8I filter papers using a BioRad 583 gel dryer. Autoradiographs were obtained after exposure of Hyperfilm-MP film (Amersham plc, U.K.) to the dried gel for either 4 h with a screen (or over night, without a screen, to obtain a sharper image). Film bands were quantitated using a BioRad GS-670 imaging laser densitometer. Percentage cross-linking was calculated by measuring the total DNA in each lane (summed density for the double-stranded [DS] and single-stranded [SS] bands) relative to the amount of cross-linked DNA (density of DS band alone). A dose-response curve was derived by plotting drug concentration against the determined percentage level of cross-linked DNA which was then analysed to determine the concentration of test compound that results in 50% cross-linked plasmid DNA (XL₅₀).

### (b) In Vitro Cytoxicity

(i) K562 Cells

K562 human chronic myeloid leukaemia cells were maintained in RPM1 1640 medium supplemented with 10% fetal calf serum and 2 mM glutamine at 37° C. in a humidified atmosphere containing 5% CO₂ and were incubated with a specified dose of drug for 1 h at 37° C. in the dark. The incubation was terminated by centrifugation (5 min, 300 g) and the cells were washed once with drug-free medium. Following the appropriate drug treatment, the cells were transferred to 96-well microtiter plates (10⁴ cells per well, 8 wells per sample). Plates were then kept in the dark at 37° C. in a humidified atmosphere containing 5% CO₂. The assay is based on the ability of viable cells to reduce a yellow soluble tetrazolium salt, 3-(4,5-dimethylthiazol-2-yl)-2,5-diphenyl-2H-tetrazolium bromide (MTT, Aldrich-Sigma), to an insoluble purple formazan precipitate. Following incubation of the plates for 4 days (to allow control cells to increase in number by approximately 10 fold), 20 μL of MTT solution (5 mg/mL in phosphate-buffered saline) was added to each well and the plates further incubated for 5 h. The plates were then centrifuged for 5 min at 300 g and the bulk of the medium pipetted from the cell pellet leaving 10-20 μL per well. DMSO (200 μL) was added to each well and the samples agitated to ensure complete mixing. The optical density was then read at a wavelength of 550 nm on a Titertek Multiscan ELISA plate reader, and a dose-response curve was constructed. For each curve, an IC₅₀ value was read as the dose required to reduce the final optical density to 50% of the control value.

(ii) NCI60 Cell Screen

The National Cancer Institute (NCI), Bethesda, Md., USA has available an in vitro cytotoxicity screen which consists of approximately 60 human tumour cell lines against which compounds are tested at a minimum of five concentrations each differing 10-fold. A 48 h continuous exposure protocol is used, where cell viability or growth is estimated with an SRB protein assay.

The test compounds were evaluated against approximately 60 human tumour cell lines. The NCI screening procedures were described in detail by Monks and co-workers (Monks, A et al., Journal of the National Cancer Institute, 1991, 83, 757). Briefly, cell suspensions were diluted according to the particular cell type and the expected target cell density (5000-40,000 cells per well based on cell growth characteristics), and added by pipette (100 μL) into 96-well microtitre plates. The cells were allowed a preincubation period of 24 h at 37° C. for stabilisation. Dilutions at twice the intended test concentration were added at time zero in 100 μL aliquots to the wells. The test compounds were evaluated at five 10-fold dilutions (10⁻⁴, 10⁻⁵, 10⁻⁶, 10⁻⁷ and 10⁻⁸ μM). The test compounds were incubated for 48 h in 5% CO₂ atmosphere and 100% humidity. The cells were then assayed using the sulphorhodamine B assay. A plate reader was used to read the optical densities and a microcomputer processed the readings into GI₅₀ values (in Moles), which is the dosage required to limit cell growth to 50%.

TABLE 1
Comparison of DNA Cross-linking and
Cytotoxicity Data for the dimers

| Compound | XL₅₀ | IC₅₀ | GI₅₀ |
|---|---|---|---|
| Number | (μM) | (μM) | (μM) |
| (15) AT-281 | N/A | 0.34 | 0.01 |
| (16) AT-242 | Alkali: 0.18 | 2.21 | 0.02 |
| (17) AT-288 | N/A | 0.52 | 0.02 |
| (2) SJG-604 | Alkali: 3.5 | 23.0 | 31.6 |
| (1) SJG-605 | Alkali: 1.3 | 1.20 | 1.00 |
| (13) AT-217 | Alkali: 0.35 | N/A | 19.0 |
| | Heat: 3.8 | | |
| (14) AT-234 | Alkali: <0.1 | 25.5 | 0.01 |
| | Heat: 0.4 | | |
| (18) AT-235 | Alkali: <0.1 | 1.51 | 0.01 |
| | Heat: 0.23 | | |
| (55) AT-338 | N/A | 0.0404 | 0.0295 |
| (63) AT-361 | N/A | 33.8 | 0.022 |

1. A compound of formula (I):
PBD-A-Y—X—(Het)ₙₐ-L-(Het)_{nb}-L-(Het)_{nc}-T-(Het′)_{nd}-L-(Het′)ₙₑ-L-(Het′)_{nf}—X′—Y′-A′-PBD′ (I)
and salts, solvates, chemically protected forms, and prodrugs thereof, wherein 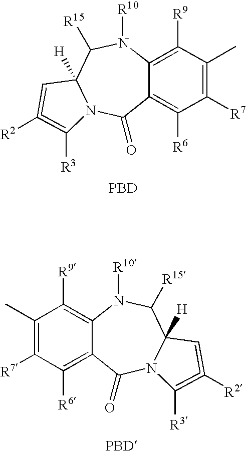
with the bonds at the 8 position on each molecule bond to the A and A′ groups respectively.
the dotted lines indicate the optional presence of a double bond between C1 and C2 or C2 and C3;
R² and R³ are independently selected from —H, —OH, ═O, ═CH₂, —CN, —R, OR, halo, ═CH—R, O—SO₂—R, CO₂R and COR;
R⁶, R⁷ and R⁹ are independently selected from H, R, OH, OR, SH, SR, NH₂, NHR, NRR′, nitro, Me₃Sn and halo; where R and R′ are independently selected from optionally substituted C₁₋₇ alkyl, C₃₋₂₀ heterocyclyl and C₅₋₂₀ aryl groups; or
R⁶ and R⁷ together form a group —O—(CH₂)ₚ—O—, where p is 1 or 2;
R¹⁰ is a nitrogen protecting group and R¹⁵ is either O—R¹¹, where R¹¹ is a hydroxyl protecting group; or
R¹⁵ is OH, ═O or ═S; or
R¹⁰ and R¹⁵ together form a double bond between C10 and N11;
A is selected from O, S, NH or a single bond;
Y is a divalent group such that HY═R, or a single bond;
X and X′ are both either NH or C(═O);
each Het and Het′ is independently an amino-heteroarylene-carbonyl group;
each L is independently selected from β-alanine, glycine, 4-aminobutanoic acid and a single bond;
T is a divalent linker group of the form:
—NH-Q-NH— or —C(═O)-Q-C(═O)—
wherein Q is a divalent group such that HQ=R;
A′, Y′, Het′, R^{2′}, R^{3′}, R^{6′}, R^{7′}, R^{9′}, R^{10′}, R^{11′}, R^{15′} and R^{15′} are all independently selected from the same lists as previously defined for A, Y, Het, R², R³, R⁶, R⁷, R⁹, R¹⁰, R¹¹ and R¹⁵ respectively;
na, nb, nc, nd, ne and nf are each independently 0 to 5 and the sum na+nb+nc+nd+ne+nf is 0 to 16.

2. A compound according to claim 1, wherein the sums na+nb+nc and nd+ne+nf are equal.

3. A compound according to claim 1, wherein Het and Het′ are nitrogen containing heteroarylene units.

4. A compound of formula (II):
PBD-A-Y—X—(Het)_{ng}-[L-(Het)ₙₕ]ₙⱼ-X′—Y′-A′-PBD′ (II)
and salts, solvates, chemically protected forms, and prodrugs thereof, wherein 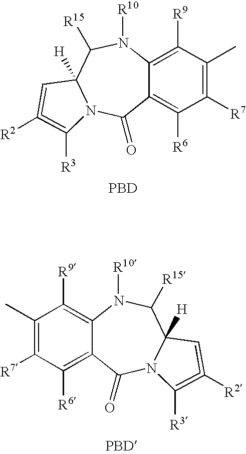
the bonds at the 8 position on PBD and PBD′ bond to A and A′ groups respectively;
the dotted lines indicate the optional presence of a double bond between C1 and C2 or C2 and C3;
R² and R³ are independently selected from —H, —OH, ═O, ═CH₂, —CN, —R, OR, halo, ═CH—R, O—SO₂—R, CO₂R and COR;
R⁶, R⁷ and R⁹ are independently selected from H, R, OH, OR, SH, SR, NH₂, NHR, NRR′, nitro, Me₃Sn and halo; where R and R′ are independently selected from optionally substituted C₁₋₇ alkyl, C₃₋₂₀ heterocyclyl and C₅₋₂₀ aryl groups; or
R⁶ and R⁷ together form a group —O—(CH₂)ₚ—O—, where p is 1 or 2;
R¹⁰ is a nitrogen protecting group and R¹⁵ is either O—R¹¹, where R¹¹ is a hydroxyl protecting group; or
R¹⁵ is OH, ═O or ═S; or
R¹⁰ and R¹⁵ together form a double bond between C10 and N11;
A is selected from O, S, NH or a single bond;
Y is a divalent group such that HY═R, or a single bond;
each Het is independently an amino-heteroarylene-carbonyl group;
each L is independently selected from β-alanine, glycine, 4-aminobutanoic acid and a single bond;
A′, Y′, R^{2′}, R^{3′}, R^{6′}, R^{7′}, R^{9′}, R^{10′}, R^{11′} and R^{15′} are all independently selected from the same lists as previously defined for A, Y, Het, R², R³, R⁶, R⁷, R⁹, R¹⁰, R¹¹ and R¹⁵ respectively;
ng is 1 to 5, nh is 1 to 5 and nj is 0 to 3
X and X′ are either NH and C(═O) respectively or C(═O) and NH respectively.

5. A compound according to claim 4, wherein the total number of Het groups in the compound represented by the sum ng+(nj×nh)) is 1 to 3.

6. A compound according to claim 4, wherein Het are nitrogen containing heteroarylene units.

7. A compound according to either claim 1 or claim 4, wherein PBD and PBD′ are the same.

8. A compound according to either claim 1 or claim 4, wherein R⁹ and R^{9′} are H.

9. A compound according to either claim 1 or claim 4, wherein R², R³, R^{2′} and R^{3′} are independently selected from R and H.

10. A compound according to either claim 1 or claim 4, wherein R⁶ and R^{6′} are independently selected from H, OH, OR, SH, NH₂, nitro and halo.

11. A compound according to either claim 1 or claim 4, wherein R⁷ and R^{7′} are independently selected from H, OR, SH, SR, NH₂, NHR, NRR′ and halo.

12. A compound according to either claim 1 or claim 4, wherein R¹⁰ and R¹⁵ together form a double bond between N10 and C11 and R^{10′} and R^{15′} together form a double bond between N10′ and C11′.

13. A compound according to either claim 1 or claim 4, wherein R¹⁰ and R^{10′} are independently selected from H, BOC, Troc and alloc, and R¹¹ and R^{11′} are independently selected from OH, THP or a silyl oxygen protecting group.

14. (canceled)

15. A pharmaceutical composition containing a compound of either claims 1 or claim 4, and a pharmaceutically acceptable carrier or diluent.

16. (canceled)

17. A method of treatment of a proliferative disease, comprising administering to a subject in need of treatment a therapeutically-effective amount of a compound of either claims 1 or claim 4.
